SEQUENCE LISTING (139 sequences)

SEQ ID NO: 1 - DNA, 32 bp, Pseudomonas aeruginosa
  source 1..32: mol_type = genomic DNA
gtcgcgccccgcacgggcgcgtggattgaaac

SEQ ID NO: 2 - DNA, 32 bp, synthetic construct
  misc_feature 1..32: note = Description of Artificial Sequence: Synthetic oligonucleotide
  source 1..32: mol_type = other DNA
gtcgcccggcaaaaccgggcgtggattgaaac

SEQ ID NO: 3 - protein, 744 aa, Pseudomonas aeruginosa
MDAEASDTHFFAHSTLKADRSDWQPLVEHLQAVARLAGEKAAFFGGGELAALAGLLHDLGKYTDEFQRRIAGDAIRVDHSTRGAILAVERYGALGQLLAYGIAGHHAGLANGREAGERTALVDRLKGVGLPRLLEGWCVEIVLPERLQPPPLKARLERGFFQLAFLGRMLFSCLVDADYLDTEAFYHRVEGRRSLREQARPTLAELRAALDRHLTEFKGDTPVNRVRGEILAGVRGKASELPGLFSLTVPTGGGKTLASLAFALDHALAHGLRRVIYVIPFTSIVEQNAAVFRRALGALGEEAVLEHHSAFVDDRRQSLEAKKKLNLAMENWDAPIVVTTAVQFFESLFADRPAQCRKLHNIAGSVVILDEAQTLPLKLLRPCVAALDELALNYRCSPVLCTATQPALQSPDFIGGLQDVRELAPEPQRLFRELVRVRIRTLGPLEDAALTEQIARREQVLCIVNNRRQARALYESLAELPGARHLTTLMCAKHRSSVLAEVRQMLKKGEPCRLVATSLIEAGVDVDFPVVLRAEAGLDSIAQAAGRCNREGKRPLAESEVLVFAAANSDWAPPEELKQFAQAAREVMRLHPDDCLSMAAIERYFRILYWQKGAEELDAGNLLGLIERGRLDGLPYETLATKFRMIDSLQLPVIIPFDDEARAALRELEFADGCAAIARRLQPYLVQMPRKGYQALREAGAIQAAAGTRYGEQFMALVNPDLYHHQFGLHWDNPAFVSSERLCW

SEQ ID NO: 4 - protein, 224 aa, Pseudomonas aeruginosa
MAYGIRLMVWGERACFTRPEMKVERVSYDAITPSAARGILEAIHWKPAIRWVVDRIQVLKPIRFESIRRNEVGGKLSAVSVGKAMKAGRTNGLVNLVEEDRQQRATTLLRDVSYVIEAHFEMTDRAGADDTVGKHLDIFNRRARKGQCFHTPCLGVREFPASFRLLEEGSAEPEVDAFLRGERDLGWMLHDIDFADGMTPHFFRALMRDGLIEVPAFRAAEDKA

SEQ ID NO: 5 - protein, 587 aa, Pseudomonas aeruginosa
MILSALNDYYQRLLERGEANISPFGYSQEKISYALLLSAQGELLDVQDIRLLSGKKPQPRLMSVPQPEKRTSGIKSNVLWDKTSYVLGVSAKGGERTQQEHESFKTLHRQILVGEGDPGLQALLQFLDCWQPEQFKPPLFSEAMLDSNLVFRLDGQQRYLHETPAALALRTRLLADGDSREGLCLVCGQRQPLARLHPAVKGVNGAQSSGASIVSFNLDAFSSYGKSQGENAPVSEQAAFAYTTVLNHLLRRDEHNRQRLQIGDASVVFWAQADTPAQVAAAESTFWNLLEPPADDGQEAEKLRGVLDAVATGRPLHELDSLMEEGTRIFVLGLAPNTSRLSIRFWAVDSLAVFTQHLAEHFRDMHLEPLPWKTEPAIWRLLYATAPSRDGRAKTEDLLPQLAGEMTRAILTGSRYPRSLLANLIMRMRADGDVSGIRVALCKAVLAREARLSGKIHQEELPMSLDKDASNPGYRLGRLFAVLEGAQRAALGDRVNATIRDRYYGAASSTPATVFPILLRNTQNHLAKLRKEKPGLAVNLERDIGEIIDGMQSQFPRCLRLEDQGRFAIGYYQQAQARFNRGPDSVE

SEQ ID NO: 6 - protein, 289 aa, Pseudomonas aeruginosa
MTAISNRYEFVYLFDVSNGNPNGDPDAGNMPRLDPETNQGLVTDVCLKRKIRNYVSLEQESAPGYAIYMQEKSVLNNQHKQAYEALGIESEAKKLPKDEAKARELTSWMCKNFFDVRAFGAVMTTEINAGQVRGPIQLAFATSIDPVLPMEVSITRMAVTNEKDLEKERTMGRKHIVPYGLYRAHGFISAKLAERTGFSDDDLELLWRALANMFEHDRSAARGEMAARKLIVFKHEHAMGNAPAHVLFGSVKVERVEGDAVTPARGFQDYRVSIDAEALPQGVSVREYL

SEQ ID NO: 7 - DNA, 32 bp, Legionella pneumophila
  source 1..32: mol_type = genomic DNA
gtcgcgccccgtgcgggcgcgtggattgaaac

SEQ ID NO: 8 - DNA, 32 bp, Desulfovibrio vulgaris
  source 1..32: mol_type = genomic DNA
gtcgccccccacgcgggggcgtggattgaaac

SEQ ID NO: 9 - DNA, 33 bp, Eggerthella lenta
  source 1..33: mol_type = genomic DNA
gtcactccccgcatggggagtgcgggttgaaat

SEQ ID NO: 10 - DNA, 40 bp, synthetic construct
  misc_feature 1..40: note = Description of Artificial Sequence: Synthetic oligonucleotide
  source 1..40: mol_type = other DNA
gaaacctgcaatgccggaggttgtagccaagttgtaattg

SEQ ID NO: 11 - DNA, 40 bp, synthetic construct
  misc_feature 1..40: note = Description of Artificial Sequence: Synthetic oligonucleotide
  source 1..40: mol_type = other DNA
ggacgttacggcctccaacatcggttcaacattaacagcg

SEQ ID NO: 12 - DNA, 40 bp, synthetic construct
  misc_feature 1..40: note = Description of Artificial Sequence: Synthetic oligonucleotide
  source 1..40: mol_type = other DNA
gaaacggtacgcaggaaaaggctctggaacaggcagttgg

SEQ ID NO: 13 - DNA, 40 bp, synthetic construct
  misc_feature 1..40: note = Description of Artificial Sequence: Synthetic oligonucleotide
  source 1..40: mol_type = other DNA
gccatgcgtccttttccgagaccttgtccgtcaaccagcg

SEQ ID NO: 14 - DNA, 21 bp, synthetic construct
  misc_feature 1..21: note = Description of Artificial Sequence: Synthetic oligonucleotide
  source 1..21: mol_type = other DNA
gcggaacggctattcccaatg

SEQ ID NO: 15 - DNA, 21 bp, synthetic construct
  misc_feature 1..21: note = Description of Artificial Sequence: Synthetic oligonucleotide
  source 1..21: mol_type = other DNA
acttcgagatccagggctacc

SEQ ID NO: 16 - DNA, 40 bp, synthetic construct
  misc_feature 1..40: note = Description of Artificial Sequence: Synthetic oligonucleotide
  source 1..40: mol_type = other DNA
gaaacggcgagtacgtagacatgccggaagaccatctcgg

SEQ ID NO: 17 - DNA, 40 bp, synthetic construct
  misc_feature 1..40: note = Description of Artificial Sequence: Synthetic oligonucleotide
  source 1..40: mol_type = other DNA
gcgaccgagatggtcttccggcatgtctacgtactcgccg

SEQ ID NO: 18 - DNA, 40 bp, synthetic construct
  misc_feature 1..40: note = Description of Artificial Sequence: Synthetic oligonucleotide
  source 1..40: mol_type = other DNA
gaaacctcggcccgctgctgcgcctgggcaactacgaacg

SEQ ID NO: 19 - DNA, 40 bp, synthetic construct
  misc_feature 1..40: note = Description of Artificial Sequence: Synthetic oligonucleotide
  source 1..40: mol_type = other DNA
gcgacgttcgtagttgcccaggcgcagcagcgggccgagg

SEQ ID NO: 20 - DNA, 22 bp, synthetic construct
  misc_feature 1..22: note = Description of Artificial Sequence: Synthetic oligonucleotide
  source 1..22: mol_type = other DNA
ctgctggtacagctcctggatg

SEQ ID NO: 21 - DNA, 22 bp, synthetic construct
  misc_feature 1..22: note = Description of Artificial Sequence: Synthetic oligonucleotide
  source 1..22: mol_type = other DNA
gcgagtacgagcacgaactgtc

SEQ ID NO: 22 - DNA, 40 bp, synthetic construct
  misc_feature 1..40: note = Description of Artificial Sequence: Synthetic oligonucleotide
  source 1..40: mol_type = other DNA
gaaacctcggctgcgccaaccaggccggcgaggacaaccg

SEQ ID NO: 23 - DNA, 40 bp, synthetic construct
  misc_feature 1..40: note = Description of Artificial Sequence: Synthetic oligonucleotide
  source 1..40: mol_type = other DNA
gcgacggttgtcctcgccggcctggttggcgcagccgagg

SEQ ID NO: 24 - DNA, 40 bp, synthetic construct
  misc_feature 1..40: note = Description of Artificial Sequence: Synthetic oligonucleotide
  source 1..40: mol_type = other DNA
gaaacagcggcaccgccgcgaggtcgtcggcgcgcaccgg

SEQ ID NO: 25 - DNA, 40 bp, synthetic construct
  misc_feature 1..40: note = Description of Artificial Sequence: Synthetic oligonucleotide
  source 1..40: mol_type = other DNA
gcgaccggtgcgcgccgacgacctcgcggcggtgccgctg

SEQ ID NO: 26 - DNA, 20 bp, synthetic construct
  misc_feature 1..20: note = Description of Artificial Sequence: Synthetic oligonucleotide
  source 1..20: mol_type = other DNA
tgactcccgacctggtctac

SEQ ID NO: 27 - DNA, 20 bp, synthetic construct
  misc_feature 1..20: note = Description of Artificial Sequence: Synthetic oligonucleotide
  source 1..20: mol_type = other DNA
cgacagggtccgtttcatcc

SEQ ID NO: 28 - DNA, 40 bp, synthetic construct
  misc_feature 1..40: note = Description of Artificial Sequence: Synthetic oligonucleotide
  source 1..40: mol_type = other DNA
gaaaccgcgacaagggcaagaacgtattgctgctgatggg

SEQ ID NO: 29 - DNA, 40 bp, synthetic construct
  misc_feature 1..40: note = Description of Artificial Sequence: Synthetic oligonucleotide
  source 1..40: mol_type = other DNA
gcgacccatcagcagcaatacgttcttgcccttgtcgcgg

SEQ ID NO: 30 - DNA, 40 bp, synthetic construct
  misc_feature 1..40: note = Description of Artificial Sequence: Synthetic oligonucleotide
  source 1..40: mol_type = other DNA
gaaacggcagcttggcgaacaccgatggcggatacccctg

SEQ ID NO: 31 - DNA, 40 bp, synthetic construct
  misc_feature 1..40: note = Description of Artificial Sequence: Synthetic oligonucleotide
  source 1..40: mol_type = other DNA
gcgacaggggtatccgccatcggtgttcgccaagctgccg

SEQ ID NO: 32 - DNA, 19 bp, synthetic construct
  misc_feature 1..19: note = Description of Artificial Sequence: Synthetic oligonucleotide
  source 1..19: mol_type = other DNA
ttcgagcaacagcgcgaac

SEQ ID NO: 33 - DNA, 18 bp, synthetic construct
  misc_feature 1..18: note = Description of Artificial Sequence: Synthetic oligonucleotide
  source 1..18: mol_type = other DNA
tcgggacacaacagctac

SEQ ID NO: 34 - DNA, 40 bp, synthetic construct
  misc_feature 1..40: note = Description of Artificial Sequence: Synthetic oligonucleotide
  source 1..40: mol_type = other DNA
gaaacctggcacgcgcccatgccgcagaacggcgcgcgcg

SEQ ID NO: 35 - DNA, 40 bp, synthetic construct
  misc_feature 1..40: note = Description of Artificial Sequence: Synthetic oligonucleotide
  source 1..40: mol_type = other DNA
gcgacgcgcgcgccgttctgcggcatgggcgcgtgccagg

SEQ ID NO: 36 - DNA, 40 bp, synthetic construct
  misc_feature 1..40: note = Description of Artificial Sequence: Synthetic oligonucleotide
  source 1..40: mol_type = other DNA
gaaacatcgaccggcgtccgctgcgggtcgccgtcggtag

SEQ ID NO: 37 - DNA, 40 bp, synthetic construct
  misc_feature 1..40: note = Description of Artificial Sequence: Synthetic oligonucleotide
  source 1..40: mol_type = other DNA
gcgactaccgacggcgacccgcagcggacgccggtcgatg

SEQ ID NO: 38 - DNA, 22 bp, synthetic construct
  misc_feature 1..22: note = Description of Artificial Sequence: Synthetic oligonucleotide
  source 1..22: mol_type = other DNA
gaggtagccactgttgttgaag

SEQ ID NO: 39 - DNA, 22 bp, synthetic construct
  misc_feature 1..22: note = Description of Artificial Sequence: Synthetic oligonucleotide
  source 1..22: mol_type = other DNA
gaaaccgtaggacgcatgattg

SEQ ID NO: 40 - DNA, 40 bp, synthetic construct
  misc_feature 1..40: note = Description of Artificial Sequence: Synthetic oligonucleotide
  source 1..40: mol_type = other DNA
gaaaccgcgaccccgccgtgcgccatgcgatgtgcgaggg

SEQ ID NO: 41 - DNA, 40 bp, synthetic construct
  misc_feature 1..40: note = Description of Artificial Sequence: Synthetic oligonucleotide
  source 1..40: mol_type = other DNA
gcgaccctcgcacatcgcatggcgcacggcggggtcgcgg

SEQ ID NO: 42 - DNA, 40 bp, synthetic construct
  misc_feature 1..40: note = Description of Artificial Sequence: Synthetic oligonucleotide
  source 1..40: mol_type = other DNA
gaaaccagcgcctgcgggtggtagatgtcgcggccctggg

SEQ ID NO: 43 - DNA, 40 bp, synthetic construct
  misc_feature 1..40: note = Description of Artificial Sequence: Synthetic oligonucleotide
  source 1..40: mol_type = other DNA
gcgacccagggccgcgacatctaccacccgcaggcgctgg

SEQ ID NO: 44 - DNA, 20 bp, synthetic construct
  misc_feature 1..20: note = Description of Artificial Sequence: Synthetic oligonucleotide
  source 1..20: mol_type = other DNA
agcctctgagcggcactttc

SEQ ID NO: 45 - DNA, 20 bp, synthetic construct
  misc_feature 1..20: note = Description of Artificial Sequence: Synthetic oligonucleotide
  source 1..20: mol_type = other DNA
agtcgtcgagccggtaatcc

SEQ ID NO: 46 - DNA, 40 bp, synthetic construct
  misc_feature 1..40: note = Description of Artificial Sequence: Synthetic oligonucleotide
  source 1..40: mol_type = other DNA
gaaacgaccgcaacccgggcgaagcggtggactggcatcg

SEQ ID NO: 47 - DNA, 40 bp, synthetic construct
  misc_feature 1..40: note = Description of Artificial Sequence: Synthetic oligonucleotide
  source 1..40: mol_type = other DNA
gcgacgatgccagtccaccgcttcgcccgggttgcggtcg

SEQ ID NO: 48 - DNA, 40 bp, synthetic construct
  misc_feature 1..40: note = Description of Artificial Sequence: Synthetic oligonucleotide
  source 1..40: mol_type = other DNA
gaaacggcgcggagcgactgggcagcggaaagcagcggcg

SEQ ID NO: 49 - DNA, 40 bp, synthetic construct
  misc_feature 1..40: note = Description of Artificial Sequence: Synthetic oligonucleotide
  source 1..40: mol_type = other DNA
gcgacgccgctgctttccgctgcccagtcgctccgcgccg

SEQ ID NO: 50 - DNA, 22 bp, synthetic construct
  misc_feature 1..22: note = Description of Artificial Sequence: Synthetic oligonucleotide
  source 1..22: mol_type = other DNA
gcaagttcgccatcgtcatgag

SEQ ID NO: 51 - DNA, 22 bp, synthetic construct
  misc_feature 1..22: note = Description of Artificial Sequence: Synthetic oligonucleotide
  source 1..22: mol_type = other DNA
gaaccgccatgcacgcattatc

SEQ ID NO: 52 - DNA, 40 bp, synthetic construct
  misc_feature 1..40: note = Description of Artificial Sequence: Synthetic oligonucleotide
  source 1..40: mol_type = other DNA
gaaacggaattgtcgcagatttgagcggaagaggacgaag

SEQ ID NO: 53 - DNA, 40 bp, synthetic construct
  misc_feature 1..40: note = Description of Artificial Sequence: Synthetic oligonucleotide
  source 1..40: mol_type = other DNA
gcgacttcgtcctcttccgctcaaatctgcgacaattccg

SEQ ID NO: 54 - DNA, 40 bp, synthetic construct
  misc_feature 1..40: note = Description of Artificial Sequence: Synthetic oligonucleotide
  source 1..40: mol_type = other DNA
gaaactcccgtcctccgcgactgcggcacgctcacagcag

SEQ ID NO: 55 - DNA, 40 bp, synthetic construct
  misc_feature 1..40: note = Description of Artificial Sequence: Synthetic oligonucleotide
  source 1..40: mol_type = other DNA
gcgactgctgtgagcgtgccgcagtcgcggaggacgggag

SEQ ID NO: 56 - DNA, 18 bp, synthetic construct
  misc_feature 1..18: note = Description of Artificial Sequence: Synthetic oligonucleotide
  source 1..18: mol_type = other DNA
cagcatctgcaggatcac

SEQ ID NO: 57 - DNA, 18 bp, synthetic construct
  misc_feature 1..18: note = Description of Artificial Sequence: Synthetic oligonucleotide
  source 1..18: mol_type = other DNA
gtgatcgtcaccgaagtc

SEQ ID NO: 58 - DNA, 40 bp, synthetic construct
  misc_feature 1..40: note = Description of Artificial Sequence: Synthetic oligonucleotide
  source 1..40: mol_type = other DNA
gaaacacggggagcggacatcgagtattaatgaacccttg

SEQ ID NO: 59 - DNA, 40 bp, synthetic construct
  misc_feature 1..40: note = Description of Artificial Sequence: Synthetic oligonucleotide
  source 1..40: mol_type = other DNA
gcgacaagggttcattaatactcgatgtccgctccccgtg

SEQ ID NO: 60 - DNA, 40 bp, synthetic construct
  misc_feature 1..40: note = Description of Artificial Sequence: Synthetic oligonucleotide
  source 1..40: mol_type = other DNA
gaaacatggaaacatggggagggccagggaaagtcaatcg

SEQ ID NO: 61 - DNA, 40 bp, synthetic construct
  misc_feature 1..40: note = Description of Artificial Sequence: Synthetic oligonucleotide
  source 1..40: mol_type = other DNA
gcgacgattgactttccctggccctccccatgtttccatg

SEQ ID NO: 62 - DNA, 19 bp, synthetic construct
  misc_feature 1..19: note = Description of Artificial Sequence: Synthetic oligonucleotide
  source 1..19: mol_type = other DNA
gttccagcagaccatcaag

SEQ ID NO: 63 - DNA, 19 bp, synthetic construct
  misc_feature 1..19: note = Description of Artificial Sequence: Synthetic oligonucleotide
  source 1..19: mol_type = other DNA
tgaaaccgggctcgataac

SEQ ID NO: 64 - DNA, 40 bp, synthetic construct
  misc_feature 1..40: note = Description of Artificial Sequence: Synthetic oligonucleotide
  source 1..40: mol_type = other DNA
gaaacctgcagcggatcgtctacgagtactgcgccgcggg

SEQ ID NO: 65 - DNA, 40 bp, synthetic construct
  misc_feature 1..40: note = Description of Artificial Sequence: Synthetic oligonucleotide
  source 1..40: mol_type = other DNA
gcgacccgcggcgcagtactcgtagacgatccgctgcagg

SEQ ID NO: 66 - DNA, 40 bp, synthetic construct
  misc_feature 1..40: note = Description of Artificial Sequence: Synthetic oligonucleotide
  source 1..40: mol_type = other DNA
gaaacaccttggcccgtgcccagggcctgggcacgccgag

SEQ ID NO: 67 - DNA, 40 bp, synthetic construct
  misc_feature 1..40: note = Description of Artificial Sequence: Synthetic oligonucleotide
  source 1..40: mol_type = other DNA
gcgactcggcgtgcccaggccctgggcacgggccaaggtg

SEQ ID NO: 68 - DNA, 18 bp, synthetic construct
  misc_feature 1..18: note = Description of Artificial Sequence: Synthetic oligonucleotide
  source 1..18: mol_type = other DNA
agcgagctggacgaaatc

SEQ ID NO: 69 - DNA, 18 bp, synthetic construct
  misc_feature 1..18: note = Description of Artificial Sequence: Synthetic oligonucleotide
  source 1..18: mol_type = other DNA
taaccgcttgcggctatc

SEQ ID NO: 70 - DNA, 40 bp, synthetic construct
  misc_feature 1..40: note = Description of Artificial Sequence: Synthetic oligonucleotide
  source 1..40: mol_type = other DNA
gaaactggaacatagccttgcggtgcgcgctggtgcggcg

SEQ ID NO: 71 - DNA, 40 bp, synthetic construct
  misc_feature 1..40: note = Description of Artificial Sequence: Synthetic oligonucleotide
  source 1..40: mol_type = other DNA
gcgacgccgcaccagcgcgcaccgcaaggctatgttccag

SEQ ID NO: 72 - DNA, 40 bp, synthetic construct
  misc_feature 1..40: note = Description of Artificial Sequence: Synthetic oligonucleotide
  source 1..40: mol_type = other DNA
gaaacgaacacgaactgatcaaaaccaccctgcccaaggg

SEQ ID NO: 73 - DNA, 40 bp, synthetic construct
  misc_feature 1..40: note = Description of Artificial Sequence: Synthetic oligonucleotide
  source 1..40: mol_type = other DNA
gcgacccttgggcagggtggttttgatcagttcgtgttcg

SEQ ID NO: 74 - DNA, 18 bp, synthetic construct
  misc_feature 1..18: note = Description of Artificial Sequence: Synthetic oligonucleotide
  source 1..18: mol_type = other DNA
ttcggcagcttctacgac

SEQ ID NO: 75 - DNA, 18 bp, synthetic construct
  misc_feature 1..18: note = Description of Artificial Sequence: Synthetic oligonucleotide
  source 1..18: mol_type = other DNA
tcgagatcctgctgaacc

SEQ ID NO: 76 - DNA, 40 bp, synthetic construct
  misc_feature 1..40: note = Description of Artificial Sequence: Synthetic oligonucleotide
  source 1..40: mol_type = other DNA
gaaacacgattgcggacatggcaggctgccgctgctggag

SEQ ID NO: 77 - DNA, 40 bp, synthetic construct
  misc_feature 1..40: note = Description of Artificial Sequence: Synthetic oligonucleotide
  source 1..40: mol_type = other DNA
gcgactccagcagcggcagcctgccatgtccgcaatcgtg

SEQ ID NO: 78 - DNA, 94 bp, synthetic construct
  misc_feature 1..94: note = Description of Artificial Sequence: Synthetic oligonucleotide
  source 1..94: mol_type = other DNA
aattcgtcgcgccccgcacgggcgcgtggattgaaacgagacctctctggacaaaggtctcgtcgcgccccgcacgggcgcgtggattgaaaca

SEQ ID NO: 79 - DNA, 94 bp, synthetic construct
  misc_feature 1..94: note = Description of Artificial Sequence: Synthetic oligonucleotide
  source 1..94: mol_type = other DNA
agcttgtttcaatccacgcgcccgtgcggggcgcgacgagacctttgtccagagaggtctcgtttcaatccacgcgcccgtgcggggcgcgacg

SEQ ID NO: 80 - DNA, 94 bp, synthetic construct
  misc_feature 1..94: note = Description of Artificial Sequence: Synthetic oligonucleotide
  source 1..94: mol_type = other DNA
aattcgtcgcgccccgcacgggcgcgtggattgaaacgagacctctctggacaaaggtctcgtcgcccggcaaaaccgggcgtggattgaaaca

SEQ ID NO: 81 - DNA, 94 bp, synthetic construct
  misc_feature 1..94: note = Description of Artificial Sequence: Synthetic oligonucleotide
  source 1..94: mol_type = other DNA
agcttgtttcaatccacgcccggttttgccgggcgacgagacctttgtccagagaggtctcgtttcaatccacgcgcccgtgcggggcgcgacg

SEQ ID NO: 82 - DNA, 45 bp, synthetic construct
  misc_feature 1..45: note = Description of Artificial Sequence: Synthetic oligonucleotide
  source 1..45: mol_type = other DNA
ctgctctgcgaggctggccgataagacagcagcaccggtttccag

SEQ ID NO: 83 - DNA, 46 bp, synthetic construct
  misc_feature 1..46: note = Description of Artificial Sequence: Synthetic oligonucleotide
  source 1..46: mol_type = other DNA
gggcggctgttccttgtcctgtgggggctacgtgagttcggagaag

SEQ ID NO: 84 - DNA, 44 bp, synthetic construct
  misc_feature 1..44: note = Description of Artificial Sequence: Synthetic oligonucleotide
  source 1..44: mol_type = other DNA
ggcccttctccgaactcacgtagcccccacaggacaaggaacag

SEQ ID NO: 85 - DNA, 49 bp, synthetic construct
  misc_feature 1..49: note = Description of Artificial Sequence: Synthetic oligonucleotide
  source 1..49: mol_type = other DNA
cttttgctggccttttgctcacataagattggcgtcccgcatcgatctc

SEQ ID NO: 86 - DNA, 44 bp, synthetic construct
  misc_feature 1..44: note = Description of Artificial Sequence: Synthetic oligonucleotide
  source 1..44: mol_type = other DNA
ctgctctgcgaggctggccgataagcgtagaacagcaccatgtc

SEQ ID NO: 87 - DNA, 44 bp, synthetic construct
  misc_feature 1..44: note = Description of Artificial Sequence: Synthetic oligonucleotide
  source 1..44: mol_type = other DNA
tgtttcaaatagccagcatccctggggaacaggcagttggaaag

SEQ ID NO: 88 - DNA, 45 bp, synthetic construct
  misc_feature 1..45: note = Description of Artificial Sequence: Synthetic oligonucleotide
  source 1..45: mol_type = other DNA
ctggaactttccaactgcctgttccccagggatgctggctatttg

SEQ ID NO: 89 - DNA, 44 bp, synthetic construct
  misc_feature 1..44: note = Description of Artificial Sequence: Synthetic oligonucleotide
  source 1..44: mol_type = other DNA
tttgctggccttttgctcacataaggctttccgtggtccagttg

SEQ ID NO: 90 - DNA, 44 bp, synthetic construct
  misc_feature 1..44: note = Description of Artificial Sequence: Synthetic oligonucleotide
  source 1..44: mol_type = other DNA
agtgctctgcaggaattcctcgagaagggagcgcacctatggac

SEQ ID NO: 91 - DNA, 32 bp, synthetic construct
  misc_feature 1..32: note = Description of Artificial Sequence: Synthetic oligonucleotide
  source 1..32: mol_type = other DNA
cggcctgttcggacaccggagcattttccccc

SEQ ID NO: 92 - DNA, 32 bp, synthetic construct
  misc_feature 1..32: note = Description of Artificial Sequence: Synthetic oligonucleotide
  source 1..32: mol_type = other DNA
aaaatgctccggtgtccgaacaggccgccttt

SEQ ID NO: 93 - DNA, 48 bp, synthetic construct
  misc_feature 1..48: note = Description of Artificial Sequence: Synthetic oligonucleotide
  source 1..48: mol_type = other DNA
ggaatccccgtcgacggtatcgatacctgaaactagaggtactcgcgc

SEQ ID NO: 94 - DNA, 92 bp, synthetic construct
  misc_feature 1..92: note = Description of Artificial Sequence: Synthetic oligonucleotide
  source 1..92: mol_type = other DNA
ctaggtcgcgccccgcacgggcgcgtggattgaaacgagacctctctggacaaaggtctcgtcgcccggcaaaaccgggcgtggattgaaac

SEQ ID NO: 95 - DNA, 92 bp, synthetic construct
  misc_feature 1..92: note = Description of Artificial Sequence: Synthetic oligonucleotide
  source 1..92: mol_type = other DNA
ctaggtttcaatccacgcccggttttgccgggcgacgagacctttgtccagagaggtctcgtttcaatccacgcgcccgtgcggggcgcgac

SEQ ID NO: 96 - DNA, 18 bp, synthetic construct
  misc_feature 1..18: note = Description of Artificial Sequence: Synthetic oligonucleotide
  source 1..18: mol_type = other DNA
tgcggtgagcatcacatc

SEQ ID NO: 97 - DNA, 18 bp, synthetic construct
  misc_feature 1..18: note = Description of Artificial Sequence: Synthetic oligonucleotide
  source 1..18: mol_type = other DNA
atacgccgctgaaactcg

SEQ ID NO: 98 - DNA, 40 bp, synthetic construct
  misc_feature 1..40: note = Description of Artificial Sequence: Synthetic oligonucleotide
  source 1..40: mol_type = other DNA
gaaacatccacgacccgaaccgtatccacggccatctggg

SEQ ID NO: 99 - DNA, 40 bp, synthetic construct
  misc_feature 1..40: note = Description of Artificial Sequence: Synthetic oligonucleotide
  source 1..40: mol_type = other DNA
gcgacccagatggccgtggatacggttcgggtcgtggatg

SEQ ID NO: 100 - DNA, 40 bp, synthetic construct
  misc_feature 1..40: note = Description of Artificial Sequence: Synthetic oligonucleotide
  source 1..40: mol_type = other DNA
gaaaccttgacctcgggtggaataccggaggcggcgccag

SEQ ID NO: 101 - DNA, 40 bp, synthetic construct
  misc_feature 1..40: note = Description of Artificial Sequence: Synthetic oligonucleotide
  source 1..40: mol_type = other DNA
gcgactggcgccgcctccggtattccacccgaggtcaagg

SEQ ID NO: 102 - DNA, 40 bp, synthetic construct
  misc_feature 1..40: note = Description of Artificial Sequence: Synthetic oligonucleotide
  source 1..40: mol_type = other DNA
gaaacgtttgggcagacggatgattaaccggattgtgacg

SEQ ID NO: 103 - DNA, 40 bp, synthetic construct
  misc_feature 1..40: note = Description of Artificial Sequence: Synthetic oligonucleotide
  source 1..40: mol_type = other DNA
gcgacgtcacaatccggttaatcatccgtctgcccaaacg

SEQ ID NO: 104 - DNA, 18 bp, synthetic construct
  misc_feature 1..18: note = Description of Artificial Sequence: Synthetic oligonucleotide
  source 1..18: mol_type = other DNA
tctgccagttcgcaaacg

SEQ ID NO: 105 - DNA, 18 bp, synthetic construct
  misc_feature 1..18: note = Description of Artificial Sequence: Synthetic oligonucleotide
  source 1..18: mol_type = other DNA
aagcgccgcattgaagtg

SEQ ID NO: 106 - DNA, 21 bp, synthetic construct
  misc_feature 1..21: note = Description of Artificial Sequence: Synthetic oligonucleotide
  source 1..21: mol_type = other DNA
cgcatcaatcggccagaatag

SEQ ID NO: 107 - DNA, 21 bp, synthetic construct
  misc_feature 1..21: note = Description of Artificial Sequence: Synthetic oligonucleotide
  source 1..21: mol_type = other DNA
gaatacgttcggccaatggag

SEQ ID NO: 108 - DNA, 20 bp, synthetic construct
  misc_feature 1..20: note = Description of Artificial Sequence: Synthetic oligonucleotide
  source 1..20: mol_type = other DNA
ccgtgcatatcggatcagtc

SEQ ID NO: 109 - DNA, 20 bp, synthetic construct
  misc_feature 1..20: note = Description of Artificial Sequence: Synthetic oligonucleotide
  source 1..20: mol_type = other DNA
gcacagccaggtcttgatac

SEQ ID NO: 110 - DNA, 20 bp, synthetic construct
  misc_feature 1..20: note = Description of Artificial Sequence: Synthetic oligonucleotide
  source 1..20: mol_type = other DNA
gtcagcaatcactcgatacc

SEQ ID NO: 111 - DNA, 18 bp, synthetic construct
  misc_feature 1..18: note = Description of Artificial Sequence: Synthetic oligonucleotide
  source 1..18: mol_type = other DNA
tcgctttgaaggcatgac

SEQ ID NO: 112 - DNA, 40 bp, synthetic construct
  misc_feature 1..40: note = Description of Artificial Sequence: Synthetic oligonucleotide
  source 1..40: mol_type = other DNA
gaaacgccagctggcgtaatagcgaagaggcccgcaccgg

SEQ ID NO: 113 - DNA, 40 bp, synthetic construct
  misc_feature 1..40: note = Description of Artificial Sequence: Synthetic oligonucleotide
  source 1..40: mol_type = other DNA
gcgaccggtgcgggcctcttcgctattacgccagctggcg

SEQ ID NO: 114 - DNA, 40 bp, synthetic construct
  misc_feature 1..40: note = Description of Artificial Sequence: Synthetic oligonucleotide
  source 1..40: mol_type = other DNA
gaaacaccctgccataaagaaactgttacccgtaggtagg

SEQ ID NO: 115 - DNA, 40 bp, synthetic construct
  misc_feature 1..40: note = Description of Artificial Sequence: Synthetic oligonucleotide
  source 1..40: mol_type = other DNA
gcgacctacctacgggtaacagtttctttatggcagggtg

SEQ ID NO: 116 - DNA, 40 bp, synthetic construct
  misc_feature 1..40: note = Description of Artificial Sequence: Synthetic oligonucleotide
  source 1..40: mol_type = other DNA
gaaacggcggtgaaattatcgatgagcgtggtggttatgg

SEQ ID NO: 117 - DNA, 40 bp, synthetic construct
  misc_feature 1..40: note = Description of Artificial Sequence: Synthetic oligonucleotide
  source 1..40: mol_type = other DNA
gcgaccataaccaccacgctcatcgataatttcaccgccg

SEQ ID NO: 118 - DNA, 20 bp, synthetic construct
  misc_feature 1..20: note = Description of Artificial Sequence: Synthetic oligonucleotide
  source 1..20: mol_type = other DNA
tgatgtgcccggcttctgac

SEQ ID NO: 119 - DNA, 20 bp, synthetic construct
  misc_feature 1..20: note = Description of Artificial Sequence: Synthetic oligonucleotide
  source 1..20: mol_type = other DNA
gaccgcttgctgcaactctc

SEQ ID NO: 120 - DNA, 40 bp, synthetic construct
  misc_feature 1..40: note = Description of Artificial Sequence: Synthetic oligonucleotide
  source 1..40: mol_type = other DNA
gaaacatcttaattttgctgacacccgcgctcatttacag

SEQ ID NO: 121 - DNA, 40 bp, synthetic construct
  misc_feature 1..40: note = Description of Artificial Sequence: Synthetic oligonucleotide
  source 1..40: mol_type = other DNA
gcgactgtaaatgagcgcgggtgtcagcaaaattaagatg

SEQ ID NO: 122 - DNA, 40 bp, synthetic construct
  misc_feature 1..40: note = Description of Artificial Sequence: Synthetic oligonucleotide
  source 1..40: mol_type = other DNA
gaaaccatctgtgccagagttgccggtagtcatcaccacg

SEQ ID NO: 123 - DNA, 40 bp, synthetic construct
  misc_feature 1..40: note = Description of Artificial Sequence: Synthetic oligonucleotide
  source 1..40: mol_type = other DNA
gcgacgtggtgatgactaccggcaactctggcacagatgg

SEQ ID NO: 124 - DNA, 40 bp, synthetic construct
  misc_feature 1..40: note = Description of Artificial Sequence: Synthetic oligonucleotide
  source 1..40: mol_type = other DNA
gaaacatcagcacaacattacctttgcgctggatgacttg

SEQ ID NO: 125 - DNA, 40 bp, synthetic construct
  misc_feature 1..40: note = Description of Artificial Sequence: Synthetic oligonucleotide
  source 1..40: mol_type = other DNA
gcgacaagtcatccagcgcaaaggtaatgttgtgctgatg

SEQ ID NO: 126 - DNA, 40 bp, synthetic construct
  misc_feature 1..40: note = Description of Artificial Sequence: Synthetic oligonucleotide
  source 1..40: mol_type = other DNA
gaaacccgtttgtggatgttcccagcggacaagcacctcg

SEQ ID NO: 127 - DNA, 40 bp, synthetic construct
  misc_feature 1..40: note = Description of Artificial Sequence: Synthetic oligonucleotide
  source 1..40: mol_type = other DNA
gcgacgaggtgcttgtccgctgggaacatccacaaacggg

SEQ ID NO: 128 - DNA, 40 bp, synthetic construct
  misc_feature 1..40: note = Description of Artificial Sequence: Synthetic oligonucleotide
  source 1..40: mol_type = other DNA
gaaacgccgctacgtcactggcaggccgggccgggtaaag

SEQ ID NO: 129 - DNA, 40 bp, synthetic construct
  misc_feature 1..40: note = Description of Artificial Sequence: Synthetic oligonucleotide
  source 1..40: mol_type = other DNA
gcgactttacccggcccggcctgccagtgacgtagcggcg

SEQ ID NO: 130 - DNA, 40 bp, synthetic construct
  misc_feature 1..40: note = Description of Artificial Sequence: Synthetic oligonucleotide
  source 1..40: mol_type = other DNA
gaaacgcgttttgcctcagaagtggtaaatgccaccacag

SEQ ID NO: 131 - DNA, 40 bp, synthetic construct
  misc_feature 1..40: note = Description of Artificial Sequence: Synthetic oligonucleotide
  source 1..40: mol_type = other DNA
gcgactgtggtggcatttaccacttctgaggcaaaacgcg

SEQ ID NO: 132 - DNA, 40 bp, synthetic construct
  misc_feature 1..40: note = Description of Artificial Sequence: Synthetic oligonucleotide
  source 1..40: mol_type = other DNA
gaaacgcctgacgcgcgccctgaaccactgccagaccaag

SEQ ID NO: 133 - DNA, 40 bp, synthetic construct
  misc_feature 1..40: note = Description of Artificial Sequence: Synthetic oligonucleotide
  source 1..40: mol_type = other DNA
gcgacttggtctggcagtggttcagggcgcgcgtcaggcg

SEQ ID NO: 134 - DNA, 40 bp, synthetic construct
  misc_feature 1..40: note = Description of Artificial Sequence: Synthetic oligonucleotide
  source 1..40: mol_type = other DNA
gaaacagtgaatacaccgtagtcgcggaagtgtctctggg

SEQ ID NO: 135 - DNA, 40 bp, synthetic construct
  misc_feature 1..40: note = Description of Artificial Sequence: Synthetic oligonucleotide
  source 1..40: mol_type = other DNA
gcgacccagagacacttccgcgactacggtgtattcactg

SEQ ID NO: 136 - DNA, 98 bp, synthetic construct
  misc_feature 1..98: note = Description of Artificial Sequence: Synthetic oligonucleotide
  source 1..98: mol_type = other DNA
gtcgcgccccgcacgggcgcgtggattgaaacnnnnnnnnnnnnnnnnnnnnnnnnnnnnnnnnnngtcgcccggcaaaaccgggcgtggattgaaac

SEQ ID NO: 137 - DNA, 51 bp, Pseudomonas aeruginosa
  source 1..51: mol_type = genomic DNA
ataaaattacaacttggctacaacctccggcattgcaggaagcatcagctt

SEQ ID NO: 138 - protein, 6 aa, synthetic construct
  REGION 1..6: note = Description of Artificial Sequence: Synthetic 6xHis tag
HHHHHH

SEQ ID NO: 139 - protein, 13 aa, synthetic construct
  REGION 1..13: note = Description of Artificial Sequence: Synthetic peptide
RSTNRAKGLEAVS

(19) United States
(12) Patent Application Publication
(10) Pub. No.: US 2026/0234609 A1
(43) Pub. Date: Aug. 13, 2026

(54) CRISPR-CAS3 FOR MAKING GENOMIC DELETIONS AND INDUCING RECOMBINATION

(71) Applicant: The Regents of the University of California, Oakland, CA (US)

(72) Inventors: Lina Maria Leon, San Francisco, CA (US); Balint Csorgo, Alameda, CA (US); Joseph Bondy-Denomy, San Mateo, CA (US)

(73) Assignee: The Regents of the University of California, Oakland, CA (US)

(21) Appl. No.: 19/545,472

(22) Filed: Feb. 20, 2026

Related U.S. Application Data
(63) Continuation of application No. 17/621,669, filed on Jun. 19, 2020.
(60) Provisional application No. 62/865,085, filed on Jun. 21, 2019.
(60) Provisional application No. 62/942,642, filed on Dec. 2, 2019.

(51) Int. Cl. C12N 15/11; C12N 9/22; C12N 15/90

(52) U.S. Cl. CPC C12N 15/11; C12N 9/22; C12N 15/907; C12N 2310/20; C12N 2800/80

(57) ABSTRACT

The present disclosure provides methods and compositions for generating deletions, inducing recombination, and for modulating gene expression in cells using type I CRISPR-Cas systems.

### CROSS REFERENCE TO RELATED APPLICATIONS

The present application is a continuation of U.S. patent application Ser. No. 17/621,669, filed Dec. 21, 2021, which is a § 371 National Phase Application of International Application No. PCT/US2020/038821, filed Jun. 19, 2020, which claims priority to U.S. Provisional Patent Application Nos. 62/865,085, filed Jun. 21, 2019, and 62/942,642, filed Dec. 2, 2019, which applications are incorporated herein by reference in their entireties.

### SEQUENCE LISTING

A Sequence Listing conforming to the rules of WIPO Standard ST.26 is submitted electronically herewith via Patent Center and is hereby incorporated by reference in its entirety. The Sequence Listing file, identified as 081906-1548662-236330US, is 183,055 bytes in size, and was created on Apr. 7, 2026.

### BACKGROUND OF THE INVENTION

CRISPR-Cas systems are a diverse group of RNA-guided nucleases (1) that defend prokaryotes against viral invaders (2, 3). Gene-editing applications have focused on Class 2 CRISPR systems (4) (i.e., Cas9 and Cas12a), but Class 1 systems hold great potential for gene editing technologies, despite being more complex (5-8). The signature gene in Class 1 Type I systems is Cas3, a 3′-5′ ssDNA helicase-nuclease enzyme that, unlike Cas9 or Cas12a, degrades target DNA processively (5, 6, 9-14).

Organisms from all domains of life contain large segments of DNA that are poorly characterized or of unknown function. In prokaryotes, these regions are often coding, and include prophages, plasmids, and mobile islands. Methods for generating rapid and programmable large genomic deletions are needed, as current approaches are inefficient (15). A methodology that allows for targeted large genomic deletions in any host, either with precisely programmed or random boundaries, would be broadly useful (16).

Type I systems are the most prevalent CRISPR-Cas systems in nature (17), which has enabled the use of endogenous CRISPR-Cas3 systems for genetic manipulation via self-targeting. This has been accomplished in Pectobacterium atrosepticum (Type I-F) (18), Escherichia coli (Type I-E) (19,20), Sulfolobus islandicus (Type I-A) (21), in various Clostridium species (Type I-B) (22-24), Lactobacillus crispatus (Type I-E) (25), Serratia sp. (Type I-F) (26), Haloarcula hispanica (Type I-B) (27), Streptococcus thermophilus (Type I-E) (28), and Zymomonas mobilis (Type I-F) (29), often being used to generate small deletions. Additionally, recent studies have repurposed Type I systems for use in human cells, including the ribonucleoprotein (RNP) based delivery (30), and plasmid-based expression (31) of a Type I-E system, the fusion of FokI nuclease to Type I-E Cascade complex for targeted editing (32), and the use of I-E and I-B systems for transcriptional modulation (33).

There is thus a need for new methods for generating programmable and rapid large scale deletions in the genomes of cells, such as deletions of over 100 kb, as well as for new approaches for efficiently inducing homology directed repair. The present disclosure satisfies these needs and provides other advantages as well.

### BRIEF SUMMARY OF THE INVENTION

In one aspect, the present disclosure provides a I-C CRISPR-Cas3 crRNA for generating deletions or inducing homology directed repair (HDR) in a cell comprising an I-C CRISPR-Cas3 system, the crRNA comprising (i) a first repeat of from 20-40 nucleotides in length comprising a first stem and a first loop; (ii) a second repeat of from 20-40 nucleotides in length comprising a second stem and a second loop; and (iii) a spacer of from 30-40 nucleotides in length located between the first and second repeats that targets a genomic locus within the cell; wherein the nucleotide sequences of the first and second repeats differ from one another at 1, 2, 3, 4, 5, 6, 7, 8, 9, 10, 11, 12, 13, 14, or 15 positions.

In some embodiments, at least 1 of the complementary base pairs formed within the first and second stems are in reversed orientation relative to one another, i.e. the base pair is in one orientation in the first stem and in the opposite orientation in the second stem. In some embodiments, 1, 2, or 3 of the complementary base pairs formed within the first and second stems are in reversed orientation relative to one another. In some embodiments, the complementary base pairs formed within the first and second stems that are in reversed orientation relative to one another are G-C base pairs. In some embodiments, the nucleotide sequences of the first and second loops differ from one another at at least 1 position. In some embodiments, the nucleotide sequences of the first and second loops differ from one another at 1, 2, or 3 positions. In some embodiments, at each of the positions at which the nucleotide sequences of the first and second loops differ, one of the loops comprises an A or a T and the other loop comprises a C or a G. In some embodiments, the nucleotide sequences of the first and second repeats differ from one another at 4, 5, 6, 7, 8, 9, 10, 11, or 12 positions. In some embodiments, one of the repeats within the crRNA is a wild-type repeat. In some embodiments, the nucleotide sequence of one of the repeats within the crRNA comprises SEQ ID NO: 1, SEQ ID NO: 2, SEQ ID NO: 7, SEQ ID NO: 8, or SEQ ID NO: 9. In some embodiments, the nucleotide sequence of one repeat within the crRNA comprises SEQ ID NO: 1, and the nucleotide sequence of the other repeat comprises SEQ ID NO: 2. In some embodiments, the crRNA is truncated by 5-15 nucleotides from the 5′ and/or the 3′ end so as to reduce the length of the first or the second repeat relative to the other repeat.

In another aspect, the present disclosure provides a I-C CRISPR-Cas3 crRNA for generating deletions or inducing HDR in a cell comprising an I-C CRISPR-Cas3 system, the crRNA consisting of (i) a sequence of from 20-40 nucleotides in length comprising a stem and a loop; and (ii) a spacer sequence of from 30-40 nucleotides in length that targets a genomic locus within the cell.

In some embodiments, the sequence comprising a stem and a loop comprises SEQ ID NO: 1, SEQ ID NO: 2, SEQ ID NO: 7, SEQ ID NO: 8, or SEQ ID NO: 9.

In another aspect, the present disclosure provides an expression cassette comprising any of the herein-described crRNAs, operably linked to a promoter.

In some embodiments, the promoter is a constitutive promoter. In some embodiments, the promoter is an inducible promoter.

In another aspect, the present disclosure provides a vector comprising any of the herein-described expression cassettes.

In another aspect, the present disclosure provides a method of inducing a deletion in the genome of a cell comprising a type I-C CRISPR-Cas3 system, the method comprising introducing into the cell a I-C CRISPR-Cas3 crRNA, wherein the introduction of the crRNA into the cell results in a deletion in the genome of the cell at the targeted genomic locus.

In some embodiments of the method, the crRNA is any of the herein-described modified crRNAs. In some embodiments, the introducing step comprises the introduction of a vector into the cell comprising a polynucleotide encoding the crRNA, operably linked to a promoter. In some embodiments, the promoter is a constitutive promoter. In some embodiments, the promoter is an inducible promoter. In some embodiments, the method further comprises contacting the cell with an agent or condition that induces expression of the crRNA in the cell. In some embodiments, the cell is a bacterial cell. In some embodiments, the I-C CRISPR-Cas3 system is endogenous to the cell. In some embodiments, the method further comprises introducing an anti-CRISPR inhibitor (aca, or anti-anti-CRISPR) into the cell. In some embodiments, the anti-CRISPR inhibitor is aca1. In some embodiments, the anti-CRISPR inhibitor is introduced by introducing a polynucleotide encoding the anti-CRISPR inhibitor, operably linked to a promoter, into the cell. In some embodiments, the polynucleotide encoding the anti-CRISPR inhibitor is present on the same vector as a polynucleotide encoding a crRNA. In some embodiments, the cell is a eukaryotic cell. In some embodiments, the cell is a mammalian cell, a fungal cell or a plant cell. In some embodiments, the cell is a human cell.

In some embodiments, the I-C CRISPR-Cas3 system is heterologous to the cell, and the method further comprises introducing the I-C CRISPR-Cas3 system into the cell. In some such embodiments, introducing the I-C CRISPR-Cas3 system into the cell comprises introducing polynucleotides encoding the Cas3, Cas5, Cas7 and Cas8 proteins into the cell, wherein the polynucleotides are operably linked to one or more promoters such that the Cas3, Cas5, Cas7 and Cas8 proteins are expressed in the cell. In some embodiments, the one or more promoters are constitutive promoters. In some embodiments, the one or more promoters are inducible promoters. In some embodiments, the method further comprises contacting the cell with an agent or condition that induces expression of the Cas3, Cas5, Cas7 and Cas8 proteins in the cell. In some embodiments, the polynucleotides encoding the Cas3, Cas5, Cas7 and Cas8 proteins are present on a single plasmid or vector. In some embodiments, the crRNA and I-C CRISPR-Cas3 system are introduced into the cell by introducing preformed RNPs comprising the Cas3, Cas5, Cas7, Cas8 proteins and the crRNA into the cell.

In some embodiments of the method, the deletion is at least 5 kb, 10 kb, 15 kb, 20 kb, 25 kb, 50 kb, 100 kb, 150 kb, 200 kb, or 250 kb in length. In some embodiments, the deletion is at least 250 kb in length. In some embodiments, a single crRNA is used to target the genomic locus. In some embodiments, more than one crRNA is introduced into the cell in order to generate multiple deletions in multiplex fashion. In some embodiments, the method does not comprise the introduction of a homologous repair template.

In some embodiments, the method further comprises the introduction of a homologous repair template into the cell, wherein the homologous repair template comprises two homologous regions that are homologous to genomic sequences flanking the targeted genomic locus, and wherein the deletion in the genome of the cell at the targeted genomic locus induced by the crRNA is repaired by homology-directed repair (HDR) using the template. In some embodiments, one or both of the homologous regions of the template is at least 500 bp long. In some embodiments, the homologous repair template is present on a plasmid. In some embodiments, the genomic regions that are homologous to the homologous regions of the template are separated by 1-20 kb, 20-40 kb, 40-60 kb, 60-80 kb, or 80-100 kb in the genome. In some embodiments, the HDR results in a deletion in the genome corresponding to the genomic sequence separating the genomic regions corresponding to the homologous regions of the template. In some embodiments, a nucleotide sequence that is present between the homologous regions of the template and that is not present in the corresponding genomic sequence is inserted into the genome, such that the HDR results in an insertion in the genome. In some embodiments, the nucleotide sequence present between the homologous regions of the template differs from the corresponding genomic sequence by at least one nucleotide, wherein the HDR results in the introduction of the nucleotide sequence present on the template into the genome, such that the HDR results in a modification of the genomic sequence.

In some embodiments, the crRNA induces deletions at an efficiency of at least 70%, 75%, 80%, 85%, 90%, 95%, or more in the genome of the cell. In some embodiments, the method is performed in vitro. In some embodiments, the method is performed in vivo. In some embodiments, the method is performed ex vivo.

In another aspect, the present disclosure provides a cell comprising a heterologous I-C CRISPR-Cas3 crRNA. In some embodiments, the heterologous I-C CRISPR-Cas3 crRNA is any of the herein-described modified crRNAs.

In another aspect, the present disclosure provides a cell comprising any of the herein-described expression cassettes or vectors.

In some embodiments, the cell further comprises a heterologous I-C CRISPR-Cas3 system. In some embodiments, the heterologous I-C CRISPR-Cas3 system comprises polynucleotides encoding the Cas3, Cas5, Cas7 and Cas8 proteins, operably linked to one or more promoters such that the Cas3, Cas5, Cas7 and Cas8 proteins are expressed in the cell. In some embodiments, the heterologous I-C CRISPR Cas-3 system comprises the Cas3, Cas5, Cas7 and Cas8 proteins. In some embodiments, the one or more promoters are constitutive promoters. In some embodiments, the one or more promoters are inducible promoters. In some embodiments, the cell is a eukaryotic cell. In some embodiments, the cell is a mammalian cell, a fungal cell, or a plant cell. In some embodiments, the cell is a human cell. In some embodiments, the cell is a bacterial cell. In some embodiments, the cell further comprises an anti-CRISPR inhibitor (aca) or a polynucleotide encoding an anti-CRISPR inhibitor. In some embodiments, the cell further comprises a homologous repair template.

In another aspect, the present disclosure provides a kit for generating deletions or inducing HDR in a cell, comprising any of the herein-described crRNAs, expression cassettes, or vectors.

In some embodiments, the kit further comprises a I-C CRISPR-Cas3 system. In some embodiments, the I-C CRISPR-Cas3 system comprises a vector comprising polynucleotides encoding the Cas3, Cas5, Cas7 and Cas8 proteins, operably linked to one or more promoters. In some embodiments, the I-C CRISPR-Cas3 system comprises the Cas3, Cas5, Cas7 and Cas8 proteins. In some embodiments, the crRNA and the Cas3, Cas5, Cas7, and Cas8 proteins are pre-assembled into RNPs. In some embodiments, the kit further comprises an anti-CRISPR inhibitor or a polynucleotide encoding an anti-CRISPR inhibitor. In some embodiments, the kit further comprises a homologous repair template.

In another aspect, the present disclosure provides a method of repressing or activating the expression of a gene in a cell, comprising (i) introducing a crRNA into the cell that targets the promoter of the gene; and (ii) introducing Cas5, Cas7, and Cas8 into the cell.

In some embodiments of the method, the Cas5, Cas7, and Cas8 are introduced into the cell by introducing a plasmid or vector comprising polynucleotides encoding Cas5, Cas7, and Cas8, operably linked to one or more promoters such that the Cas5, Cas7, and Cas8 proteins are expressed in the cell. In some embodiments, the one or more promoters are constitutive promoters. In some embodiments, the one or more promoters are inducible promoters. In some embodiments, the method further comprises contacting the cell with an agent or condition that induces expression of the Cas5, Cas7, and Cas8 proteins in the cell. In some embodiments, the crRNA, Cas5, Cas7, and Cas8 are introduced into the cell by introducing pre-formed RNPs comprising the Cas5, Cas7, Cas8 proteins and the crRNA. In some embodiments, the method is used to activate the expression of the gene, and one or more of the Cas5, Cas7, or Cas8 proteins are fusion proteins comprising a transcriptional activator. In some such embodiments, the transcriptional activator is VP64. In some embodiments, the method is used to repress the expression of the gene, and one or more of the Cas5, Cas7, or Cas8 proteins are fusion proteins comprising a transcriptional repressor. In some such embodiments, the transcriptional repressor is KRAB.

### BRIEF DESCRIPTION OF THE DRAWINGS

FIGS. 1A-1D. FIG. 1A: A schematic of the Type I-C cas gene operon and CRISPR array. The surveillance complex is made up of Cas proteins (Cas5 (1): Cas8 (1): Cas7 (7)) and one crRNA, which recruits Cas3 upon target DNA recognition. FIG. 1B: Growth curves of 2 PAO1^{IC} strains expressing different crRNAs targeting phzM (green and orange) compared to a non-targeting strain (blue). Values are the mean of 8 biological replicates each, error bars indicate SD values. FIG. 1C: Cultures resulting from phzM targeting, in the absence of inducer (−ind), presence (+ind), and after recovery. FIG. 1D: Whole-genome sequencing of three PAO1^{IC} self-targeted survivor strains. Bars indicate boundaries of deletions; red arrow indicates genomic position of targeted sequences.

FIGS. 2A-2E. FIG. 2A: Percentage of survivors with a genomic deletion at the location targeted. Six different crRNA constructs with either wild-type (Wt) repeat sequences (light green) or with the second repeat being modified (dark green). Values are means of 3 biological replicates each, where 12 individual surviving colonies were assayed per replicate, error bars show SD values. FIG. 2B: Sequence and structure of natural and modified repeat sequences. Specifically engineered modified nucleotides shown in red; repeat sequences highlighted in gray with an arbitrary intervening spacer sequence. FIG. 2C: Growth curves of PAO1^{IC} strains expressing distinct self-targeting crRNAs flanked by modified repeats. Non-targeting crRNA expressing control is marked in blue. Values depicted are averages of 4 biological replicates each. FIG. 2D: Gene editing outcomes for distinct survivor cells targeted with either a Type II-A SpyCas9 system or a Type I-C Cas3 system (n=72). FIG. 2E: Percentage of survivors with the specific deletion size present (0.17 kb, 56.5 kb, or 249 kb) using homologous repair templates with the Cas3 system (green) or the SpyCas9 system (blue). Values are means of 3 biological replicates each, where 12 individual surviving colonies were assayed per replicate, error bars show SD values, ND: not detected.

FIGS. 3A-3C. FIG. 3A: Schematic overview of the iterative deletion generating process. FIG. 3B: Whole-genome sequences of six PAO1^{IC} strains that have been iteratively targeted at six distinct genomic positions and one (derived from strain Δ6 (6)) with ten total deletions (Δ10) aligned to the parental P. aeruginosa PAO1^{IC} strain. The first six targeted sites are marked with red arrows, and the final four are marked with blue arrows. FIG. 3C: Calculated doubling times of the seven genome-reduced strains (strains Δ61-66 with six deletions, Δ10 with ten) compared to the parent PAO1^{IC} strain (green). Values are means of 8 biological replicates, error bars represent SD values, * p<0.05, ** p<0.01, paired T-test compared to PAO1^{IC}.

FIGS. 4A-4F. FIG. 4A: Schematic of the crRNA targeted sites in the E. coli MG1655 genome at the lacZ locus. FIG. 4B: lacZ deletion efficiencies using distinct crRNAs targeting the E. coli K-12 MG1655 chromosome. Efficiencies calculated based on LacZ activity. Values are averages of 3 biological replicates, error bars represent standard deviations. FIG. 4C: Whole-genome sequencing of an E. coli deletion mutant targeted 30 kb upstream of lacZ at pdeL. FIG. 4D: Growth of P. syringae DC3000 strains expressing the I-C system and distinct crRNAs. Constructs VI, IV-IX, and VIII target P. syringae DC3000 non-essential chromosomal genes, non-targeting crRNA (NT), empty vector (EV). FIG. 4E: Bacterial growth of deletion mutants in Arabidopsis thaliana. Values are differences in colony forming units (cfu)/ml counted on day 0 of the experiment and day 3, shown on a logarithmic scale. The wild-type DC3000 strain is shown in black, while gray bars represent previously constructed polymutant control (C) strains of the different clusters (labeled at bottom), and green and blue bars show deletion mutants generated using Cas3 (two isolated strains for each targeted cluster, #1, #2). Values shown are means of 10 biological replicates each (30 for DC3000), error bars show SD values, ** p<0.01, *** p<0.005, ANOVA analysis (see methods). FIG. 4F: Whole-genome sequencing of P. syringae deletion mutants. Left panel shows virulence cluster VI targeting, while right panel shows virulence cluster IV and IX targeting with a single crRNA, as the clusters share sequence identity.

FIGS. 5A-5C. FIG. 5A: Schematic of whole genome sequencing of an environmental isolate of PAO1 with an endogenous Type I-C system. Two survivors were isolated post-targeting using either WT direct repeats flanking the spacer, or modified repeats. FIG. 5B: Editing efficiencies at targeted genomic sites using homologous templates in a laboratory (PA14) and clinical (z8) strain of P. aeruginosa. See Table 3 for additional details. FIG. 5C: Growth curves of PAO1^{IC} lysogenized by recombinant DMS3m phage expressing acrIIA4 or acrIC1 from the native acr locus. CRISPR-Cas3 activity is induced with either 0.5 mM (+) or 5 mM (++) IPTG and 0.1% (+) or 0.3% (++) arabinose. Edited survivors reflect number of isolated survivor colonies missing the targeted gene (phzM). Each growth curve is the average of 10 biological replicates and error bars represent SD.

FIG. 6. PCR amplification of a 3 kb genomic fragment flanking the phzM gene targeted using two different crRNAs, phzM_1 and phzM_2. Colony PCRs were performed on 18 biological replicates of self-targeted strains for each crRNA. The PAO1^{IC} parental strain is used as a positive control (wt). L indicates a 1 kb DNA ladder.

FIGS. 7A-7C. FIG. 7A: Phage targeting assays with survivors that had no discernable deletion of the crRNA-targeted genomic site. Strains were transformed with a D3 phage-targeting crRNA to assay for IC CRISPR-Cas3 activity. Three unique survivors were isolated from six self-targeting assays for a total of 18 survivors. Control is a non-targeting crRNA. FIG. 7B: Schematic of spacer excision events where the two direct repeats recombine, resulting in the loss of the targeting spacer. FIG. 7C: PCR amplification of the crRNA sequence from plasmids isolated from 17 non-deletion self-targeted survivors. Pl indicates the original plasmid as the PCR template, Ni indicates a sample where the crRNA was not induced, L indicates a 1 kb DNA ladder.

FIGS. 8A-8B. FIG. 8A: Phage-targeting assay showing the activity of the modified repeat crRNA constructs. Ten-fold serial dilutions of DMS3 phage and D3 phage were spotted on lawns of PAO1^{IC} expressing either empty vector (top), a crRNA targeting D3 with WT direct repeats (middle), or a crRNA targeting D3 with modified repeats (bottom). FIG. 8B: Phage targeting assay of five non-deletion self-targeting survivors expressing a D3 phage targeting crRNA. Unsuccessful targeting of phage indicates a non-functional CRISPR-Cas system in these strains. The parental PAO1^{IC} strain with a functional CRISPR-Cas system was used as a control.

FIGS. 9A-9B. FIG. 9A: Growth curves of 36 PAO1^{IC} biological replicates targeting the essential gene, rplQ, using the MR crRNA plasmid. FIG. 9B: Phage targeting assays with eight isolated rplQ-targeted survivors to assay for I-C CRISPR-Cas activity. Serial dilutions of DMS3 phage and D3 phage were spotted on lawns of PAO1^{IC} expressing a crRNA targeting phage D3. The parent PAO1^{IC} strain expressing a D3 targeting crRNA (top left) was used as a positive control, while PAO1^{IC} expressing a non-targeting crRNA was used as a negative control.

FIG. 10. Growth of self-targeting strains of PAO1″IA expressing a self-targeting crRNA targeting the genome at phzM (Ind.). An empty vector (E.V.) and a non-induced phzM targeting strain (N.I.) were used as controls. Mean OD values measured at 600 nm are shown for 8 biological replicates each, error bars indicate SD values.

FIGS. 11A-11C. Testing of strains expressing various mutant constructs for self-targeting activity (ST) using a spacer targeting 200 bp upstream of phzM. FIG. 11A: Primers flanking the protospacer (indicated by crRNA) and phzS were used to determine deletion boundaries. FIG. 11B: Table detailing fraction of ST survivors that had a positive PCR band for the indicated region. FIG. 11C: Phage targeting activity of strains expressing the various mutant constructs. Activity was induced using 1 mM IPTG and 0.1% arabinose with a phage-targeting (T) spacer or non-targeting (NT) spacer. Higher levels of induction (T*) were used in one case (5 mM IPTG and 0.1% arabinose). WT indicates the wild-type I-C system, while Cas3-Cas8 denotes the tethered construct of Cas3 fused to the Cascade complex via Cas8.

FIG. 12. Schematic overview of the generation of deletions with predetermined coordinates of various sizes. Sequences with ~400 bp homology to genomic sites (purple and yellow boxes for the short deletion, red and orange boxes for the long deletion) were cloned into the vector crRNA vector.

FIG. 13. Deletion efficiencies observed over six cycles of iterative self-targeting. Six genomic targets were targeted in six different orders. Six survivors were analyzed using site-specific PCR after each cycle, for a total of 36 analyzed colonies (6*6) after each cycle.

FIGS. 14A-14D. FIG. 14A: Map of the I-C CRISPR-Cas all-in-one plasmid pCas3cRh carrying I-C crRNA and genes cas3, cas5, cas8, and cas7 under the control of the rhamnose-inducible rhaSR-PrhaBAD system. FIG. 14B: Growth curve of PAO1 transformed with the pCas3cRh vector expressing a self-targeting crRNA targeting phzM (Ind.). An empty vector (E.V.) and a non-induced phzM targeting strain (N.I.) were used as controls. Mean OD values measured at 600 nm are shown for six biological replicates each. FIG. 14C: Deletion efficiencies for WT PAO1 using the all-in-one vector pCas3cRh carrying all necessary components of the I-C CRISPR-Cas system. Values are averages of three replicates where 12 individual colonies were analyzed using site-specific PCR. Error bars show standard deviations. FIG. 14D: Transformation efficiencies with self-targeting pCas3cRh vectors expressing crRNAs for phzM or XNES 2 compared to a non-targeting control (green bar) in PAO1. Values are means of 3 replicates each, error bars represent SD values.

FIGS. 15A-15G. FIG. 15A: Percentage of survivors with targeted deletions in clusters of non-essential virulence effector genes in P. syringae pv. tomato DC3000. Values are averages of three biological replicates where 12 individual colonies were analyzed using site-specific PCR for each, error bars show standard deviations. FIG. 15B: In vitro growth of cluster VI deletion strains in King's medium B (KB). ΔCEL is the previously published polymutant, while ΔCVI-1 and ΔCVI-2 are Cas3-generated mutants. Error bars represent standard deviation, n=4. FIG. 15C: In vitro growth of cluster IV, cluster IX deletion strains in KB. ΔCEL is the previously published polymutant, while ΔCIVΔCIX-1 and ΔCIVΔCIX-2 are Cas3-generated mutants. Error bars represent standard deviation, n=4. FIG. 15D: In vitro growth of cluster X deletion strains in KB. ΔCEL is the previously published polymutant, while ΔCX-1 and ΔCX-2 are Cas3-generated mutants. Error bars represent standard deviation, n=4. FIG. 15E: In vitro growth of cluster VI deletion strains in apoplast mimicking minimal media (MM). ΔCEL is the previously published polymutant, while ΔCVI-1 and ΔCVI-2 are Cas3-generated mutants. Error bars represent standard deviation, n=4. FIG. 15F: In vitro growth of cluster IV, cluster IX deletion strains in MM. ΔCEL is the previously published polymutant, while ΔCIVΔCIX-1 and ΔCIVΔCIX-2 are Cas3-generated mutants. Error bars represent standard deviation, n=4. FIG. 15G: In vitro growth of cluster X deletion strains in MM. ΔCEL is the previously published polymutant, while ΔCX-1 and ΔCX-2 are Cas3-generated mutants. Error bars represent standard deviation, n=4.

FIGS. 16A-16C. FIG. 16A: Editing efficiencies for the Pseudomonas aeruginosa environmental isolate naturally expressing the Type I-C cas genes, transformed with a plasmid targeting phzM with WT repeats or modified repeats. Each data point represents the fraction of isolates with the deletion out of ten isolates assayed. FIG. 16B: Genotyping results for the Pseudomonas aeruginosa environmental isolate using the 0.17 kb HDR template. Larger band corresponds to the WT sequence, smaller band corresponds to a genome reduced by 0.17 kb. FIG. 16C: Genotyping results of PAO1^{IC} AcrC1 lysogens after self-targeting induction in the presence or absence of aca1 and a non-targeted control. Ten biological replicates per strain were assayed. gDNA was extracted from each replicate and PCR analysis for the phzM gene (targeted gene, top row of gels) or cas5 gene (non-targeted gene, bottom row) was conducted. Only cells that co-expressed aca1 with the crRNA showed loss of the phzM band, indicating genome editing. All replicates had a cas5 band, indicating successful gDNA extraction and target specificity for the phzM locus.

FIGS. 17A-17B. Determination of deletion size distribution. FIG. 17A: Schematic representation of tiling PCR experiment to determine distribution of deletion sizes when targeting the genome of Pseudomonas aeruginosa strain PAO1IC using a crRNA specific to phzM gene. Single colonies were isolated from a total of 47 cultures targeted in parallel and analyzed using colony PCR amplifying 7 different fragments at various distances from the targeted genomic site. Absence or presence of the given fragments for each sample allowed the determination of the range of the size of the deletion. Genes depicted in red are essential genes that cannot be deleted from the genome, blue arrows show the relative positions of the primer pairs. FIG. 17B: Distribution of the minimal deletion sizes within the 47 analyzed colonies at the targeted genomic site.

FIGS. 18A-18D. Cas3 editing in Klebsiella pneumoniae strain KPPR1. 4 crRNAs were individually expressed from the pCas3cRh plasmid to target the genome of Klebsiella pneumoniae strain KPPR1 (2 crRNAs each targeting rfaH and sacX genes). FIG. 18A: Induced expression of self-targeting crRNAs resulted in significant growth delays compared to a non-targeting crRNA (blue). Values are the mean of 8 biological replicates each, error bars indicate SD values. FIG. 18B: Percentage of survivors with targeted deletions at rfaH and sacX genes in Klebsiella pneumoniae KPPR1. Each gene was targeted using two different crRNAs (1 and 2). Values are averages of three biological replicates where 8 individual colonies were analyzed using site-specific PCR for each, error bars show standard deviations. FIG. 18C: Representative gel electrophoresis runs of PCR reaction products of self-targeted Klebsiella Pneumoniae KPPR1 cells (8 colonies tested for each targeting crRNA, wt indicates wild-type control, M indicates marker ladder). FIG. 18D: KPPR1 strain with presumed deletion at rfaH gene shows smaller colony size compared to wild-type, as described previously (Bachman, M. A. et al. Genome-Wide Identification of Klebsiella pneumoniae Fitness Genes during Lung Infection. mBio 6, (2015)), indicating successful deletions taking place.

FIG. 19. Editing efficiencies of nuclease mutant Cas3, helicase mutant Cas3, and Cas3-Cas8 tethered construct. Testing of strains expressing various mutant constructs for self-targeting activity using a spacer targeting 200 bp upstream of phzM. Primers flanking the protospacer (indicated by crRNA) and phzS were used to determine deletion boundaries (schematic drawing at bottom) Graph shows various editing efficiencies of nuclease mutant Cas3, helicase mutant Cas3, and Cas3-Cas8 tethered construct.

### DETAILED DESCRIPTION OF THE INVENTION

### 1. Introduction

The present disclosure is based on the discovery that a reduced-complexity CRISPR-Cas3 subtype, hereby referred to as Type I-C, which employs the Cas3 dual helicase-nuclease enzyme (distinct from Cas9), can be adapted for bacterial and eukaryotic genome editing to enable the generation of both random and predetermined large deletions (>1 kb and up to and exceeding 250 kb), at up to high (e.g., 95%) efficiency. The methods described herein can also be used to combine multiple, e.g., 10 or more, deletions in a single host, leading to, e.g., an over 13% total genome reduction in a selected bacterium. Additionally, multiplex targeting of various genomic loci can allow the simultaneous deletion of distinct regions within the same cell. Type I CRISPR-Cas3 systems are the most common immune systems found in sequenced bacterial genomes, and we have developed CRISPR-Cas3 as an endogenous editing technique to provide novel tools for both genetically intractable and tractable organisms, including both prokaryotes and eukaryotes. The disclosure is also based on the discovery that the deletions induced in the present methods are also highly recombinogenic, and can be used to induce HDR-mediated insertions, deletions, and modifications of the genome in the presence of a homologous repair template.

The present disclosure provides methods and compositions for using the CRISPR-Cas3 system as a tool for genomic manipulations that lack CRISPR-Cas3 systems naturally. The system is portable to other bacteria, e.g., introducing and expressing heterologous CRISPR-Cas3 system components, and to eukaryotic cells as well, including fungi, vertebrates, and mammals including humans. The methods and compositions are also based in part on the discovery that editing efficiency can be dramatically enhanced by modifying the RNA sequences in the CRISPR RNA (crRNA). CRISPR-Cas systems utilize short RNAs in a specific fold in complex with Cas proteins to base-pair with the complementary target sequence that is to be edited. These RNAs are encoded between repetitive DNA elements. The present disclosure provides mutated forms of the repetitive sequences to 1) disrupt homology between the repeats while 2) maintaining the proper RNA fold. Without being bound by the following theory, it is believed that the lack of perfect homology between the repeats, together with the maintained RNA fold of each repeat, prevents or reduces recombination events between the repeats and results in higher editing efficiency. Forms of the crRNA in which one of the repeats is absent, or in which one or both repeats are truncated, are also provided. Overall, the CRISPR-Cas3 technology described herein can be used for rapid bacterial and eukaryotic engineering for, e.g., synthetic biological and metabolic engineering purposes.

The present disclosure also provides methods of using the I-C CRISPR-Cas3 system for gene repression or activation. In particular, the components of the system without Cas3 itself, e.g., comprising Cas5, Cas7, and Cas8, can be directed by crRNAs to specific gene targets in cells and repress or activate their expression, in the absence of the helicase-nuclease activity provided by Cas3. In some of these cases, one or more of the Cas5, Cas7, and Cas8 can be linked to a transcriptional repressor (such as KRAB) or activator (such as VP64).

### 2. General

Practicing this invention utilizes routine techniques in the field of molecular biology. Basic texts disclosing the general methods of use in this invention include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255:137-149 (1983).

### 3. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter. In some embodiments, the promoter is a prokaryotic promoter, e.g., a promoter used to drive crRNA, anti-anti-CRISPR, or I-C CRISPR-Cas3 gene expression in prokaryotic cells. Typical prokaryotic promoters include elements such as short sequences at the −10 and −35 positions upstream from the transcription start site, such as a Pribnow box at the −10 position typically consisting of the six nucleotides TATAAT, and a sequence at the −35 position, e.g., the six nucleotides TTGACA.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a heterologous promoter. In the context of promoters operably linked to a polynucleotide, a “heterologous promoter” refers to a promoter that would not be so operably linked to the same polynucleotide as found in a product of nature (e.g., in a wild-type organism).

As used herein, a first polynucleotide or polypeptide is “heterologous” to an organism or a second polynucleotide or polypeptide sequence if the first polynucleotide or polypeptide originates from a foreign species compared to the organism or second polynucleotide or polypeptide, or, if from the same species, is modified from its original form. For example, when a promoter is said to be operably linked to a heterologous coding sequence, it means that the coding sequence is derived from one species whereas the promoter sequence is derived from another, different species; or, if both are derived from the same species, the coding sequence is not naturally associated with the promoter (e.g., is a genetically engineered coding sequence).

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “expression” and “expressed” refer to the production of a transcriptional and/or translational product, e.g., of a crRNA and/or a nucleic acid sequence encoding a protein (e.g., a I-C CRISPR-Cas3 system component or an anti-anti-CRISPR). In some embodiments, the term refers to the production of a transcriptional and/or translational product encoded by a gene (e.g., a cas3, cas5, cas7, or cas8 gene) or a portion thereof. The level of expression of a DNA molecule in a cell may be assessed on the basis of either the amount of corresponding mRNA that is present within the cell or the amount of protein encoded by that DNA produced by the cell.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of an I-C CRISPR-Cas3 protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine(S), Threonine (T); and
8) Cysteine (C), Methionine (M)
(see, e.g., Creighton, Proteins, W. H. Freeman and Co., N. Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

A “I-C CRISPR-Cas3 system” refers to a class 1 CRISPR-Cas system, comprising a multi-subunit crRNA-effector complex, more specifically to a type I system, and even more specifically to a subtype I-C system. Subtype I-C systems can comprise a number of different Cas components, including Cas1, Cas2, Cas3, Cas4, Cas5, Cas7, and Cas8 (e.g., Cas8c) (see, e.g., Makarova et al. (2015) Nat. Rev. Microbiol. 13, 722-736 (2015)), although I-C CRISPR-Cas3 systems as used herein often comprise systems with minimal components, e.g., Cas3, Cas5, Cas7 and Cas8. Further, as described elsewhere herein, systems can be used that lack Cas3, e.g., for gene repression or activation purposes, i.e., systems comprising Cas5, Cas7, and Cas8 alone, while still being considered a I-C CRISPR-Cas3 system. While in particular embodiments the Cas proteins used in the present methods are derived from prokaryotes with native I-C systems, e.g., Pseudomonas aeruginosa, it will be understood that Cas proteins or genes, e.g., Cas3, Cas5, Cas7 or Cas8 proteins, or cas3, cas5, cas7, or cas8 genes, can be used from any source, including from prokaryotes with a CRISPR-Cas system other than a subtype I-C system. The Cas polypeptides and polynucleotides used in the present methods and compositions include wild-type Cas genes and proteins and fragments and variants thereof, e.g., Cas3, Cas5, Cas7 and Cas8 proteins or cas3, cas5, cas7, and cas8 genes, or polynucleotides or polypeptides having 50%, 60%, 70%, 80%, 90%, 95%, 96%, 97%, 98%, 99%, or greater homology to wild-type Cas genes or proteins or fragments or variants thereof. In particular embodiments, the Cas3 protein used in the methods comprises the sequence shown as SEQ ID NO:3 or a fragment thereof, or a polynucleotide is used encoding SEQ ID NO:3 or a fragment thereof, or a polypeptide is used with 50%, 60%, 70%, 80%, 90%, 95%, 96%, 97%, 98%, 99%, or greater homology to SEQ ID NO:3 or a fragment thereof, or a polynucleotide is used that encodes a polypeptide with 50%, 60%, 70%, 80%, 90%, 95%, 96%, 97%, 98%, 99% or greater homology to SEQ ID NO:3 or a fragment thereof. In particular embodiments, the Cas5 protein used in the methods comprises the sequence shown as SEQ ID NO:4 or a fragment thereof, or a polynucleotide is used encoding SEQ ID NO:4 or a fragment thereof, or a polypeptide is used with 50%, 60%, 70%, 80%, 90%, 95%, 96%, 97%, 98%, 99%, or greater homology to SEQ ID NO:4 or a fragment thereof, or a polynucleotide is used that encodes a polypeptide with 50%, 60%, 70%, 80%, 90%, 95%, 96%, 97%, 98%, 99% or greater homology to SEQ ID NO:4 or a fragment thereof. In particular embodiments, the Cas8 protein used in the methods comprises the sequence shown as SEQ ID NO:5 or a fragment thereof, or a polynucleotide is used encoding SEQ ID NO:5 or a fragment thereof, or a polypeptide is used with 50%, 60%, 70%, 80%, 90%, 95%, 96%, 97%, 98%, 99%, or greater homology to SEQ ID NO:5 or a fragment thereof, or a polynucleotide is used that encodes a polypeptide with 50%, 60%, 70%, 80%, 90%, 95%, 96%, 97%, 98%, 99% or greater homology to SEQ ID NO:5 or a fragment thereof. In particular embodiments, the Cas7 protein used in the methods comprises the sequence shown as SEQ ID NO:6 or a fragment thereof, or a polynucleotide is used encoding SEQ ID NO:6 or a fragment thereof, or a polypeptide is used with 50%, 60%, 70%, 80%, 90%, 95%, 96%, 97%, 98%, 99%, or greater homology to SEQ ID NO:6 or a fragment thereof, or a polynucleotide is used that encodes a polypeptide with 50%, 60%, 70%, 80%, 90%, 95%, 96%, 97%, 98%, 99% or greater homology to SEQ ID NO:6 or a fragment thereof. Information about the structure, sequences, function, and other properties of type I-C systems, including I-C CRISPR-Cas3 system proteins as well as I-C CRISPR-Cas3 system crRNAs, can be found, e.g., in Hochstrasser et al. (2016) Molecular Cell 63:840-851; Nam et al. (2012) Structure 20:1574-1584; Rao et al., (2016) Cellular Microbiology doi: 10.111/cmi.12586; Makarova et al. (2011) Nature Reviews Microbiology 9:467-477; Makarova et al. (2015) Nature Reviews Microbiology 13:722-736; and in the online database TIGRFAM (ftp.jcvi.org/pub/data/TIGRFAMs/); the disclosures of each of which is herein incorporated by reference in its entirety.

The crRNAs, or CRISPR RNAs, or “I-C CRISPR-Cas3 CRNAs” used herein can be any crRNA that can function with an endogenous or exogenous I-C CRISPR-Cas3 system to direct the induction of deletions, HDR, or gene activation or repression in cells. The crRNAs can be bound by the proteins of an I-C CRISPR-Cas3 system, e.g., Cas3, Cas5, Cas7 and/or Cas8. As used herein, an “I-C CRISPR-Cas3 crRNA” refers to a crRNA that, when incubated together with one or more proteins of a I-C CRISPR-Cas3 system, e.g., Cas3, Cas5, Cas7, and/or Cas8, is bound by the one or more I-C CRISPR-Cas3 system proteins and can direct the proteins to a target (genomic or extragenomic) DNA sequence as defined by (e.g., being complementary or homologous to) the spacer sequence of the crRNA. A “I-C CRISPR-Cas3 crRNA” can also be any naturally occurring, or “wild-type,” crRNA that is present in a CRISPR array in any species with a I-C CRISPR-Cas3 system, or to a crRNA made using a repeat sequence from any CRISPR array from any species with a I-C CRISPR-Cas3 system (e.g., as shown in SEQ ID NOS: 1, 7, 8, and 9). crRNAs comprise a spacer sequence of, e.g., 30, 31, 32, 33, 34, 35, 36, 37, 38, 39, or 40 nucleotides in length, or 32-37 nucleotides in length, with homology to the targeted genomic or extragenomic sequence at a position adjacent to a Type I-C CRISPR PAM sequence (e.g., 5′-TTC-3′), as well as one or more repeat sequences of, e.g., 10, 11, 12, 13, 14, 15, 16, 17, 18, 19, 20, 21, 22, 23, 24, 25, 26, 27, 28, 29, 30, 31, 32, 33, 34 or more nucleotides in length, or e.g., 20-40, 30-40, 25-35 nucleotides in length, comprising a stem-loop structure. It will be understood that spacer sequences can have less than 30 nucleotides, e.g., 15, 20, 25, 15-20, 20-25, or 25-30 nucleotides. Exemplary wild-type crRNA repeat sequences are provided herein as SEQ ID NO: 1, SEQ ID NO: 7, SEQ ID NO: 8, and SEQ ID NO: 9. crRNAs can also be modified, e.g., in the stem region, the loop region, or outside of the stem-loop region, as described elsewhere herein and as shown, e.g., in SEQ ID NO: 2, which includes exchanged complementary base pairs within the stem region and modified nucleotides within the loop. For example, in crRNAs that comprise two repeats, the repeat sequences can differ from one another at one or more nucleotides, e.g., 1, 2, 3, 4, 5, 6, 7, 8, 9, 10, 11, 12, 13, 14, 15, or more nucleotides, as described in more detail elsewhere herein. crRNAs can also comprise a single repeat sequence together with the spacer sequence, e.g., a single repeat sequence as shown in SEQ ID NO: 1, SEQ ID NO: 2, or SEQ ID NOS: 7-9, and can also comprise truncations within one or both repeat sequences, e.g., a truncation of 1, 2, 3, 4, 5, 6, 7, 8, 9, 10, or greater nucleotides from the 5′ or 3′ end of the crRNA, e.g., a truncation relative to a full-length crRNA, e.g., a full-length wild-type crRNA. The overall length of the crRNA can vary and is typically, e.g., 60-120 nucleotides in length, e.g., 60, 70, 80, 90, 100, 110, 120, or any integer within that range, or e.g., 60-90, 60-100, 60-110, 70-120, 80-120, 90-120, or 70-100, 80-100, or 90-100 nucleotides in length.

A homologous repair template refers to a polynucleotide sequence that can be used to repair a double stranded break (DSB) in the DNA, e.g. a break as induced using the herein-described methods and compositions. The homologous repair template comprises homology to the genomic sequence surrounding the DSB, e.g., a crRNA target sequence of the invention. In some embodiments, two distinct homologous regions are present on the template, with each region comprising at least 50, 100, 200, 300, 400, 500, 600, 700, 800, 900 or more nucleotides or more of homology with the corresponding genomic sequence. In some embodiments, the homologous regions correspond to genomic regions that are separated by, e.g., at least 50, 100, 200, 300, 400, 500, 600, 700, 800, 900 bp, or 1, 5, 10, 20, 30, 40, 50, 60, 70, 80, 90, 100 kb or more in the genome. The repair template can be present in any form, e.g. on a plasmid that is introduced into the cell, as a free floating doubled stranded DNA template (e.g., a template that is liberated from a plasmid in the cell), or as single stranded DNA. As the sequence separating the homologous regions on the template will be introduced into the genome by HDR, the present methods can be used to induce precisely defined deletions into the genome (i.e., if the genomic sequence normally present between the homologous regions is absent on the template), to introduce insertions (i.e., if a nucleotide sequence that is not normally present in the genome at the corresponding genomic locus is present on the template between the homologous regions), or to introduce modifications to the genome (i.e., if the nucleotide sequence between the homologous regions on the template differs from the corresponding genomic sequence at one or more nucleotides).

“Anti-CRISPR inhibitor”, or “anti-anti-CRISPR,” or “anti-CRISPR-associated” (Aca) proteins, or (aca) genes, refers to a family of genes and encoded proteins that are associated with, e.g., downstream of within the same operon, Anti-CRISPR loci. Aca proteins contain Helix-Turn-Helix (HTH) domains and bind to acr promoters, typically to the inverted repeats within acr promoters, and repress transcription of the acr coding sequence. Acas include, but are not limited to, Aca1, Aca2, Aca3, Aca4, Aca5, Aca6, Aca7, Aca8, or AcrIIA1 family members, variants, derivatives, or fragments, e.g., the NTD domain, thereof from any species, as well as polynucleotides or polypeptides sharing at least 50%, 60%, 70%, 80%, 20 90%, 95%, 96%, 97%, 98%, 99%, to any of these Acas or acas. It will be understood that any aca gene associated with any acr locus from any species, i.e., a sequence coding for an HTH-containing polypeptide that is capable of binding to the acr locus and inhibiting its transcription, is encompassed by the present methods.

### 4. Detailed Description of the Embodiments

### Deletions, HDR, and Gene Repression or Activation

The present disclosure provides novel methods and compositions for generating deletions and inducing HDR in cells and for targeted gene repression or activation using the I-C CRISPR-Cas3 system. In particular embodiments, the present methods and compositions allow for the introduction of one or more crRNAs into cells to target nucleic acid sequences, e.g., genomic sequences or extra-genomic sequences such as plasmid sequences, and thereby generate deletions that include and extend from the specific nucleic acid sequence(s) targeted by the one or more crRNAs. The deletions generated can be of any size, e.g., about 5 kb, 10 kb, 15 kb, 20 kb, 25 kb, 50 kb, 75 kb, 100 kb, 150 kb, 200 kb, 250 kb, or larger, or up to, e.g., 100 kb, 150 kb, 200 kb, 250 kb, 300 kb, 350 kb, 400 kb, 450 kb, or 500 kb, or up to any size that permits survival of the cell. The deletions can be made in a semi-random fashion, e.g., extending from the genomic target site and creating deletions of unpredicted length, or can be made in a more precise fashion in conjunction with the use of a homologous repair template. The methods and compositions can be used in any cell type, including bacterial cells that do or do not have an endogenous I-C CRISPR-Cas3 system, and eukaryotic cells including fungi, vertebrates, plants, and mammals including humans.

In some embodiments, the methods and compositions are used to generate deletions or induce HDR in a cell, e.g., a bacterial cell, that comprises an endogenous I-C CRISPR-Cas3 system. In such embodiments, the methods comprise, e.g., the introduction of an exogenous crRNA to target a specific site within the genomic or extragenomic DNA and generate a deletion extending from the specific site, or introducing a deletion, insertion, or modification by HDR as defined by a homologous repair template that is also introduced into the cell. In some embodiments, an anti-anti-CRISPR is also introduced into the cell to inhibit any present, or potentially present, CRISPR inhibitors in the cell. Any genomic or extragenomic site can be targeted using the herein provided crRNAs, as long as there is an appropriate type I PAM site adjacent to the targeted sequence. Any of the herein-described crRNAs can be used in such methods, including crRNAs with naturally occurring, i.e., wild-type, repeat sequences, or crRNAs with modified repeat sequences as described herein, crRNAs with truncated repeat sequences, or crRNAs with absent repeat sequences such that the crRNA comprises only one repeat sequence and the spacer sequence. In any such methods, a single crRNA can be introduced into a cell to generate a single deletion (or multiple deletions, if the targeted sequence is found in more than one genomic or extragenomic location), or multiple crRNAs can be introduced, e.g., as many as 10 or more crRNAs, simultaneously or in succession to generate multiple deletions in multiplex fashion.

In other embodiments, the methods and compositions are used to generate deletions or induce HDR in a cell, e.g., a bacterial cell, fungal cell, vertebrate cell, mammalian cell, human cell, that does not contain an endogenous I-C CRISPR-Cas3 system. In such embodiments, a “portable” I-C CRISPR-Cas3 system, e.g., comprising Cas3, Cas5, Cas7, and Cas8, or comprising polynucleotides encoding Cas3, Cas5, Cas7, and Cas8, operably linked to one or more promoters, is introduced into the cell in conjunction with one or more crRNAs to direct the Cas3-mediated induction of deletions at genomic or extragenomic sites as directed by the crRNA spacer sequence. In some embodiments, the I-C CRISPR-Cas3 system is introduced into the cell using a plasmid or other vector comprising polynucleotides encoding the Cas3, Cas5, Cas7, and Cas8 proteins, operably linked to one or more promoters, such that the Cas3, Cas5, Cas7, and Cas8 are expressed in the cell. In other embodiments, the Cas3, Cas5, Cas7, and Cas8 proteins are introduced directly into the cell, e.g., as individual proteins, as a protein complex, or as a ribonucleoprotein (RNP), i.e., a pre-formed protein-crRNA complex comprising the proteins and a crRNA. As noted herein, in aspects where the goal is repression or activation of expression rather than deletion, Cas3 can be omitted. In some embodiments, a deletion, insertion, or genomic modification is induced by introducing a homologous repair template into the cell together with the crRNA and IC CRISPR-Cas3 system.

The present disclosure provides methods and compositions for inducing deletions, insertions, and modifications of the genome using HDR. In such methods, a deletion is induced using an I-C CRISPR-Cas3 system and a crRNA targeting a specific site within the genome, and a homologous repair template is introduced comprising homology to genomic sequence surrounding the targeted site. In some embodiments, the homologous repair template is used to introduce precisely defined deletions in the genome, e.g., the homologous regions on the template correspond to genomic sequences separated by, e.g., about 100, 200, 300, 400, 500, 600, 700, 800, 900 bp or more, or by about 1, 5, 10, 20, 30, 40, 50, 60, 70, 80, 90, 100 kb or more, and the genomic region normally present between the genomic sequences is absent on the template, such that the intervening region between the genomic sequences is deleted from the genome when the double stranded break induced by the I-C CRISPR-Cas3 system is repaired by HDR. In some embodiments, the homologous repair template is used to insert a sequence into the targeted genomic site, e.g., a sequence is present on the template between the homologous regions that is not normally present at the corresponding genomic locus, such that the sequence is introduced into the genome when the double stranded break induced by the I-C CRISPR-Cas3 system is repaired by HDR. In some embodiments, the homologous repair template is used to modify the genomic sequence at the targeted genomic site, e.g., the nucleotide sequence present on the template between the homologous regions differs from the corresponding genomic sequence at one or more nucleotides, such that the sequence present on the template is introduced into the genome when the double stranded break induced by the I-C CRISPR-Cas3 system is repaired by HDR.

The homologous repair template can be present, e.g., on a plasmid, as free-floating DNA (e.g., as liberated from a plasmid in the cell), or as single-stranded DNA, and can be introduced before, at the same time as, or after the introduction of the I-C CRISPR-Cas3 system, anti-anti-CRISPR, and/or crRNA into the cell.

The methods and compositions can be used to generate deletions or induce HDR for any purpose. For example, deletions can be generated for the engineering of cells, e.g., bacterial strains or eukaryotic cells, to specifically or semi-randomly remove specific genes in the cells or to generate large-scale deletions. For example, deletions can be generated for use as a biological discovery tool, e.g. by targeting genomic regions of unknown function in prokaryotic or eukaryotic cells and examining the phenotypes generated by the deletions in order to determine the role that the regions play. In addition, the methods could be used to obtain genetically streamlined mutants that are optimized for maximal yield of a product of interest.

In higher-order eukaryotic cells such as plants and animals, for example, Cas3-based editing can be used as an unbiased discovery tool for dissecting the role of genomic “dark matter.” For example, the human genome is composed of ~98% non-coding DNA, much of which remains functionally uncharacterized. While Cas9 as a DNA deletion tool is limited in its ability to interrogate the large amounts of dark matter in the human genome, because it mostly generates very small (<20 bp) insertions and deletions at its target site, employing Cas3 to make large genomic deletions can facilitate the manipulation of repetitive and non-coding regions.

In some embodiments, the methods and compositions are used to target cells, in vitro or in vivo, for destruction or genomic modification by directing an endogenous or exogenous I-C CRISPR-Cas3 system to specific genomic or extragenomic, e.g., plasmid-based, targets. For example, pathogenic or other undesired cells can be selectively killed through the introduction of a crRNA targeting an essential genomic site or region that is specific to the pathogenic or undesired cells, such that an endogenous or exogenous I-C CRISPR-Cas3 system is directed to generate lethal deletions in the cell. Such genomic targets could be, for example, an essential gene, or could reside in a region of the genome that contains one or more essential genes that are likely be encompassed by deletions generated using the methods. In some embodiments, antimicrobial resistant bacteria are targeted by introducing one or more crRNAs targeting the antimicrobial resistance (AMR) locus, such that the resistant cells are selectively killed and/or AMR-containing plasmids are destroyed.

In particular embodiments, a deletion, e.g., a large deletion of 25 kb, 50 kb, 75 kb, 100 kb, 150 kb, 200 kb, 250 kb, or larger, is generated using a single crRNA, taking advantage of the combined helicase-nuclease activity of Cas3. This is in contrast to, e.g., Cas9-based methods, where, e.g., two guide RNAs may be used at the extremities of the region to be deleted. Accordingly, the present methods are advantageous in that they are both simpler to use, with the introduction of only a single crRNA, and simpler to design, with the sole need for the generation of a single crRNA sequence as opposed to two different sequences. In addition, deletions can be generated in a semi-random fashion, with no need to define or determine ahead of time the precise limits of the genomic region to be deleted. Further, while homologous repair templates can be used in the context of the present disclosure to generate precisely defined deletions, the present methods can also be used to generate deletions without a homologous repair template, as may be required, e.g., with Cas9-based systems.

In some embodiments, a I-C CRISPR-Cas3 system that lacks Cas3 is used to selectively repress gene expression as targeted by a crRNA specific to the gene, e.g., the promoter of the gene. For example, a I-C CRISPR system can be introduced into cells, e.g., comprising Cas5, Cas7, and Cas8 but without Cas3, together with one or more crRNAs. In such embodiments, the system can be introduced by introducing a vector comprising polynucleotides encoding the proteins of the system, e.g., Cas5, Cas7, and Cas8 into cells, by introducing the proteins of the system directly into the cells, or by introducing a ribonucleoprotein (RNP) comprising the system proteins and the crRNA that is pre-formed prior to the introduction step. In some embodiments, one or more proteins of the system can be modified so as to enhance the gene repression effect, e.g., expressed as a fusion protein with known transcription inhibitors such as KRAB.

In some embodiments, a I-C CRISPR-Cas3 system that lacks Cas3 is used to selectively activate gene expression as targeted by a crRNA specific to the gene, e.g., the promoter of the gene. For example, a I-C CRISPR system can be introduced into cells, e.g., comprising Cas5, Cas7, and Cas8 but without Cas3, together with one or more crRNAs. In such embodiments, the system can be introduced by introducing a vector comprising polynucleotides encoding the proteins of the system, e.g., Cas5, Cas7, and Cas8 into cells, by introducing the proteins of the system directly into the cells, or by introducing an RNP comprising the system proteins and the crRNA that is pre-formed prior to the introduction step. In some embodiments, one or more proteins of the system can be modified so as to enhance the gene activation effect, e.g., expressed as a fusion protein with known transcription activators such as VP64.

In some embodiments, e.g., when using the present methods and compositions to induce deletions or HDR in a prokaryotic cell with an endogenous I-C CRISPR-Cas3 system, an anti-anti-CRISPR, such as aca1, is introduced into the cell in coordination with the crRNA. In some embodiments, the anti-anti-CRISPR is introduced as a polynucleotide encoding the anti-anti-CRISPR, operably linked to a promoter, such that the anti-anti-CRISPR is expressed in the cell. In some embodiments, the polynucleotide encoding the anti-anti-CRISPR is present on the same vector as a polynucleotide encoding a crRNA. The anti-anti-CRISPR can be introduced before, at the same time as, or after the introduction of the crRNA.

Using the present methods, a single crRNA can be introduced into a cell in order to induce a single deletion or repress or activate a single gene, or a crRNA array can be introduced that comprises 2, 3, 4, 5, 6, 7, 8, 9, 10 or more crRNAs, so as to simultaneously target multiple genomic sites for deletion or for gene repression or activation.

The present methods for generating deletions and for activating or repressing gene expression can be used for other type I CRISPR-Cas systems such as subtype I-F. As such, the present disclosure also provides I-F CRISPR-Cas3 crRNAs, including modified I-F CRISPR-Cas3 crRNAs as described herein, as well as expression cassettes, vectors, and cells comprising I-F CRISPR-Cas3 crRNAs. In some embodiments, a I-F CRISPR-Cas3 crRNA is provided that comprises only one repeat sequence, with one or more truncated repeats, or with two repeats that are different at, e.g., 1, 2, 3, 4, 5, 6, 7, 8, 9, 10, 11, 12, 13, 14, 15, or more nucleotides, as described herein for I-C CRISPR-Cas3 system crRNAs. In some embodiments, the repeats contain one or more reversed complementary base pairs within the stems of the repeats. In some embodiments, the repeats contain one or more differences in the loop regions of the repeats. In some embodiments, the repeats contain one or more nucleotide differences in the repeats outside of the stem-loop. In some embodiments, methods are provided for introducing a I-F CRISPR-Cas3 crRNA, including heterologous naturally occurring crRNAs and modified crRNAs as described herein, into a cell containing an endogenous or heterologous I-C CRISPR-Cas3 system in order to induce a deletion or to activate or repress gene expression. Any of the methods or compositions described herein can be adapted and used with a I-F CRISPR-Cas3 crRNA (i.e., a naturally occurring I-F CRISPR-Cas3 crRNA, or a crRNA that can bind to I-F CRISPR-Cas3 proteins) and/or with endogenous or heterologous I-F CRISPR-Cas3 systems to induce deletions or to activate or repress gene expression.

### I-C CRISPR-Cas3 Systems

In some embodiments of the present disclosure, a I-C CRISPR-Cas3 system is introduced into a cell that does not contain an endogenous system. In such embodiments, any number of type I-C components, from any source, can be used, so long that a specified genomic or extragenomic site is targeted for deletion, HDR, or gene repression upon the introduction of a crRNA specific for the site to be deleted or repressed and optionally a homologous repair template. For example, Cas3, Cas5, Cas8 (e.g., Cas8c), Cas7, Cas4, Cas1, and Cas2 proteins, or any combination thereof, can be introduced, or polynucleotides encoding the Cas proteins, operably linked to one or more promoters, can be introduced. In particular embodiments, in particular for inducing deletions or inducing HDR, a minimal I-C CRISPR-Cas3 system is introduced comprising Cas3, Cas5, Cas8 and Cas7. In other embodiments, in particular for inducing gene repression, a system lacking Cas3 is introduced. For example, a system comprising Cas5, Cas8, and Cas7 can be introduced into cells, but without introducing Cas3.

The I-C CRISPR-Cas3 proteins used in the methods, e.g., Cas3, Cas5, Cas7 and Cas8, can be obtained from any source, including from prokaryotes with a native I-C CRISPR-Cas3 system (e.g., Pseudomonas aeruginosa, Geobacter sulfurreducens, Bacillus halodurans, Legionella pneumophila), e.g., as shown in SEQ ID NOS: 3-6. It will be understood, however, that one or more of the proteins, or polynucleotides encoding the proteins, can be obtained from other prokaryotes without a native I-C CRISPR-Cas system, e.g., from prokaryotes with another Type I CRISPR-Cas system. In particular embodiments, the I-C Cas proteins, or polynucleotides encoding the proteins, are from Pseudomonas aeruginosa. In some embodiments, a polypeptide or polynucleotide comprising, e.g., 50%, 60%, 70%, 80%, 90%, 95%, 96%, 97%, 98%, 99%, or greater homology to a I-C CRISPR-Cas3 system protein (e.g., as shown in SEQ ID NO: 3-6) or polynucleotide (e.g., a polynucleotide encoding SEQ ID NOS: 3-6), or a fragment or variant thereof, is used.

In some such embodiments, a plasmid or other vector, e.g. lentiviral vector, is introduced into a prokaryotic or eukaryotic cell containing polynucleotides encoding Cas3, Cas5, Cas8 and Cas7, operably linked to one or more promoters, such that the Cas3, Cas5, Cas8 and Cas7 proteins are expressed in the cell. In other embodiments, a vector is introduced into a cell containing polynucleotides encoding Cas5, Cas8, and Cas7, operably linked to one or more promoters, such as the Cas5, Cas8, and Cas7 proteins are expressed in the cell. In other embodiments, the Cas proteins, e.g., Cas5, Cas8, and Cas7, and with or without Cas3, are produced in vitro and either introduced directly into the cells or are used to assemble RNPs comprising the Cas proteins and a crRNA which are then introduced into the cells using standard methods and as described elsewhere herein. In some embodiments, the plasmid or other vector, or an additional plasmid, vector, or single-stranded or double-stranded DNA molecule, comprising a homologous repair template is introduced as well.

In some embodiments, the polynucleotides are present on a plasmid, and the minimal system is introduced into a bacterial cell. In other embodiments, the polynucleotides are present on a vector, e.g., a lentiviral vector, and the minimal system is introduced into eukaryotic, e.g., mammalian, cells. In particular embodiments, a plasmid or vector is introduced that includes both the minimal I-C CRISPR-Cas3 system (i.e., polynucleotides encoding Cas3, Cas5, Cas7 and Cas8), as well as one or more crRNAs, targeting one or more genomic or extragenomic sites in the cell. In such embodiments, the polynucleotides encoding the crRNA and/or I-C CRISPR-Cas3 system components are linked to one or more promoters capable of effecting expression of the crRNA and/or I-C CRISPR-Cas3 system components in the cell, including promoters for use in prokaryotic or eukaryotic cells, and including constitutive and inducible promoters.

crRNAs

The introduction of crRNAs into cells containing endogenous or heterologous I-C CRISPR-Cas3 systems is provided. The crRNAs contain a spacer sequence of, e.g., 32-37 nucleotides in length, e.g., 34 nucleotides, that is complementary to the genomic or extragenomic site to be targeted, e.g., a genomic or extragenomic site adjacent to a type I PAM sequence, as well as repeat sequences that flank the spacer sequences and that comprise sequences that give rise to stem and loop structures. Spacer sequences can be less than 30 nucleotides, e.g., less than 15, 15-20, 20-25, or 25-30 nucleotides. In wild-type CRISPR-Cas3 systems, the repeat sequences are identical, or virtually identical to one another, although in the present methods modified repeat sequences can also be used, as described in more detail elsewhere herein. Exemplary wild-type repeat sequence for use in the present methods and compositions are shown as SEQ ID NOS: 1, 7, 8 and 9. Repeats that are 50%, 60%, 70%, 80%, 90%, 95%, 96%, 97%, 98%, 99% or more identical to SEQ ID NOS: 1, 2, 7, 8 or 9, or to a fragment of SEQ ID NO: 1, 2, 7, 8 or 9, or that differ at, e.g., 1, 2, 3, 4, 5, 6, 7, 8, 9, or 10 nucleotides, can also be used.

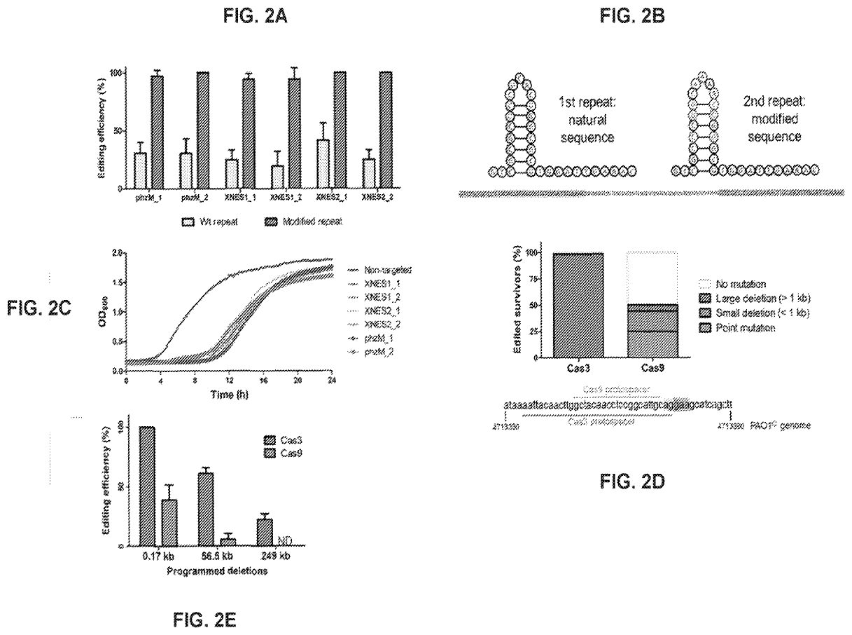

Full-length repeat sequences within the crRNAs can be, e.g., from 30-40 nucleotides in length, e.g., 30, 31, 32, 33, 34, 35, 36, 37, 38, 39, or 40 nucleotides, and contain sequences that can give rise to a stem-loop, i.e., where the RNA can fold upon itself and form hybridized base pairs between two complementary regions to form the stem, with the nucleotides located between the two complementary regions and which therefore do not hybridize to form base pairs forming the loop. For example, in SEQ ID NOS: 1 and 2, a 19 nucleotide region that starts at position 3 in the sequences forms 7 base pairs within the stem and a loop of five nucleotides (see, e.g., FIG. 2B). When a base pair in the stem is said to be in “reversed orientation”, that means that the nucleotides involved in the base pair are the same, but that the sequence is changed such that the positions of the bases are reversed. For example, in FIG. 2B, the fourth base pair within the stem of the wild-type repeat (shown as “1^{st} repeat: natural sequence” in FIG. 2B) is G-C, whereas the equivalent base pair within the stem of the modified repeat (shown as “2^{nd} repeat: modified sequence” in FIG. 2B) is C-G. The stem-loop regions of the present crRNAs can be of any length, e.g., from 10, 11, 12, 13, 14, 15, 16, 17, 18, 19, 20, 21, 22, 23, 24, 25, 26, 27, 28, 29, 30 or more nucleotides, and can contain stems containing 2, 3, 4, 5, 6, 7, 8, 9, 10 or more complementary base pairs. The loops can also be of different lengths, e.g., 2, 3, 4, 5, 6, 7, 8, 9, 10 or more nucleotides. The sequence within the repeat but outside of the stem-loop can also be of various length, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides.

In particular embodiments, a modified crRNA is used, in which one or both of the repeat sequences surrounding a spacer is modified, truncated, or absent so that the two repeats are not identical. In particular embodiments, one or both repeats are modified while still maintaining the stem and loop structures in at least one repeat. In some embodiments, the repeat sequences differ by 1 or more nucleotides, e.g., 1, 2, 3, 4, 5, 6, 7, 8, 9, 10, 11, 12, 13, 14, 15, 16, 17, 18, 19, 20 or more (e.g., 1-15, 1-8, 1-4, 2-6, 3-6) nucleotides. In some embodiments, one of the repeats flanking the spacer is a wild-type or naturally occurring sequence, and the other repeat is a modified sequence. In other embodiments, both of the repeats surrounding a spacer are modified compared to wild-type. In some embodiments, one of the repeats is absent, so that the crRNA comprises (1) a single repeat sequence comprising a stem-loop and (2) a spacer. In some embodiments, one or both repeats is truncated, e.g., from the 5′ or 3′ end of the crRNA, so as to reduce the overall length of the crRNA. In such embodiments, the truncation can remove 1, 2, 3, 4, 5, 6, 7, 8, 9, 10 or more (e.g., 15, 1-8, 1-10, 2-5) nucleotides from the 3′ and/or 5′ end of the crRNA, as compared to a full-length repeat as shown in, e.g., SEQ ID NO: 1, SEQ ID NO: 2, or SEQ ID NOS: 7-9.

In particular embodiments, nucleotides are modified in the stem region and/or the loop region of the repeat. For example, the orientation of base pairs that are formed within the stem region can be reversed, e.g., a G-C base pair could be reversed in one of the stems so that it is C-G in the stem of the other repeat. Such base-pair reversals can be implemented in, e.g., 1, 2, 3, 4, 5 or more base pairs formed within the stem. In particular embodiments, 3 base pairs are reversed, i.e., involving the introduction of 6 nucleotide differences between the two repeat sequences. In certain embodiments, nucleotides within the loop region can be modified. For example, one or more C or G within the loop region can be replaced with an A or T in one of the repeats. Such loop nucleotide changes could be implemented in, e.g., 1, 2, 3, 4 or more (e.g., 1-4, 1-3, 2-4) nucleotides within the loop. In particular embodiments, 3 nucleotides are modified. In some embodiments, repeat nucleotides outside of the stem-loop region are modified so as to differ between the two repeats. In particular embodiments, 3 base pairs within the stem region are in reversed orientation in the two repeats, and 3 nucleotides within the loop are different between the repeats, for a total of 9 total differences in the nucleotide sequences of the two repeats. In particular embodiments, one of the repeats has a wild-type sequence, e.g., as shown in SEQ ID NO: 1, or SEQ ID NOS: 7-9, and/or one of the repeats has a modified sequence, e.g., as shown in SEQ ID NO: 2.

The modification principles described herein for modifying I-C CRISPR crRNAs, e.g., involving the use of crRNAs with only one repeat sequence, with truncated repeat sequences, or with two repeat sequences containing one or more nucleotide differences, e.g., in the stem, loop, or outside of the stem-loop, can also be used in other CRISPR systems, including other type I systems (e.g., subtypes I-A, I-B, I-U, I-D, I-E, I-F) as well as in type V and type VI systems. As such, in some embodiments, the present disclosure provides a modified crRNA from a type I (e.g., type I-F), type V, or type VI CRISPR system, wherein one or both of the repeat sequences surrounding a spacer is modified, truncated, or absent so that the two repeats are not identical.

### RNA and Protein Preparation

The I-C CRISPR-Cas3 system and/or anti-anti-CRISPR polypeptides can be generated by any method. For example, in some embodiments the protein can be purified from naturally-occurring sources, synthesized, or more typically can be made by recombinant production in a cell engineered to produce the protein. Exemplary expression systems include various bacterial, yeast, insect, and mammalian expression systems.

The I-C CRISPR-Cas3 system and/or anti-anti-CRISPR polypeptides as described herein can be fused to one or more fusion partners and/or heterologous amino acids to form a fusion protein. Fusion partner sequences can include, but are not limited to, amino acid tags, non-L (e.g., D-) amino acids or other amino acid mimetics to extend in vivo half-life and/or protease resistance, targeting sequences or other sequences. In some embodiments, functional variants or modified forms of the I-C CRISPR-Cas3 system or anti-anti-CRISPR proteins include fusion proteins of a I-C CRISPR-Cas3 system or anti-anti-CRISPR polypeptides and one or more fusion domains. Exemplary fusion domains include, but are not limited to, polyhistidine, Glu-Glu, glutathione S transferase (GST), thioredoxin, protein A, protein G, an immunoglobulin heavy chain constant region (Fc), maltose binding protein (MBP), and/or human serum albumin (HSA). A fusion domain or a fragment thereof may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QLAexpress™ system (Qiagen) useful with (HIS6) fusion partners. As another example, a fusion domain may be selected so as to facilitate detection of the I-C CRISPR-Cas3 system or anti-anti-CRISPR polypeptide. Examples of such detection domains include the various fluorescent proteins (e.g., GFP) as well as “epitope tags,” which are usually short peptide sequences for which a specific antibody is available. Epitope tags for which specific monoclonal antibodies are readily available include FLAG, influenza virus haemagglutinin (HA), and c-myc tags. In some cases, the fusion domains have a protease cleavage site, such as for Factor Xa or Thrombin, which allows the relevant protease to partially digest the fusion proteins and thereby liberate the recombinant proteins therefrom. The liberated proteins can then be isolated from the fusion domain by subsequent chromatographic separation. In certain embodiments, a I-C CRISPR-Cas3 system or anti-anti-CRISPR protein is fused with a domain that stabilizes the I-C CRISPR-Cas3 system or anti-anti-CRISPR protein in vivo (a “stabilizer” domain). By “stabilizing” is meant anything that increases serum half-life, regardless of whether this is because of decreased destruction, decreased clearance by the kidney, or other pharmacokinetic effect. Fusions with the Fc portion of an immunoglobulin are known to confer desirable pharmacokinetic properties on a wide range of proteins. See, e.g., US Patent Publication No. 2014/056879. Likewise, fusions to human serum albumin can confer desirable properties. Other types of fusion domains that may be selected include multimerizing (e.g., dimerizing, tetramerizing) domains and functional domains (that confer an additional biological function, as desired). Fusions may be constructed such that the heterologous peptide is fused at the amino terminus of a I-C CRISPR-Cas3 system or anti-anti-CRISPR polypeptide and/or at the carboxyl terminus of a I-C CRISPR-Cas3 system or anti-anti-CRISPR polypeptide. In some embodiments, a fusion protein comprises a I-C CRISPR-Cas3 system polypeptide fused to a transcriptional activator (e.g., VP64) or repressor (e.g., KRAB).

In some embodiments, the I-C CRISPR-Cas system or anti-anti-CRISPR polypeptides as described herein comprise at least one non-naturally encoded amino acid. In some embodiments, a polypeptide comprises 1, 2, 3, 4, or more unnatural amino acids. Methods of making and introducing a non-naturally-occurring amino acid into a protein are known. See, e.g., U.S. Pat. Nos. 7,083,970; and 7,524,647. The general principles for the production of orthogonal translation systems that are suitable for making proteins that comprise one or more desired unnatural amino acid are known in the art, as are the general methods for producing orthogonal translation systems.

A non-naturally encoded amino acid is typically any structure having any substituent side chain other than one used in the twenty natural amino acids. Because non-naturally encoded amino acids typically differ from the natural amino acids only in the structure of the side chain, the non-naturally encoded amino acids form amide bonds with other amino acids, including but not limited to, natural or non-naturally encoded, in the same manner in which they are formed in naturally occurring polypeptides. However, the non-naturally encoded amino acids have side chain groups that distinguish them from the natural amino acids. For example, R optionally comprises an alkyl-, aryl-, acyl-, keto-, azido-, hydroxyl-, hydrazine, cyano-, halo-, hydrazide, alkenyl, alkynl, ether, thiol, seleno-, sulfonyl-, borate, boronate, phospho, phosphono, phosphine, heterocyclic, enone, imine, aldehyde, ester, thioacid, hydroxylamine, amino group, or the like or any combination thereof. Other non-naturally occurring amino acids of interest that may be suitable for use include, but are not limited to, amino acids comprising a photoactivatable cross-linker, spin-labeled amino acids, fluorescent amino acids, metal binding amino acids, metal-containing amino acids, radioactive amino acids, amino acids with novel functional groups, amino acids that covalently or noncovalently interact with other molecules, photocaged and/or photoisomerizable amino acids, amino acids comprising biotin or a biotin analog, glycosylated amino acids such as a sugar substituted serine, other carbohydrate modified amino acids, keto-containing amino acids, amino acids comprising polyethylene glycol or polyether, heavy atom substituted amino acids, chemically cleavable and/or photocleavable amino acids, amino acids with an elongated side chains as compared to natural amino acids, including but not limited to, polyethers or long chain hydrocarbons, including but not limited to, greater than about 5 or greater than about 10 carbons, carbon-linked sugar-containing amino acids, redox-active amino acids, amino thioacid containing amino acids, and amino acids comprising one or more toxic moiety.

Another type of modification that can optionally be introduced into a I-C CRISPR-Cas3 system or anti-anti-CRISPR protein (e.g. within the polypeptide chain or at either the N- or C-terminal), e.g., to extend in vivo half-life, is PEGylation or incorporation of long-chain polyethylene glycol polymers (PEG). Introduction of PEG or long chain polymers of PEG increases the effective molecular weight of the present polypeptides, for example, to prevent rapid filtration into the urine.

In certain embodiments, specific mutations of a I-C CRISPR-Cas3 system or anti-anti-CRISPR polypeptide can be made to alter the glycosylation of the polypeptide. Such mutations may be selected to introduce or eliminate one or more glycosylation sites, including but not limited to, O-linked or N-linked glycosylation sites as recognized by eukaryotic expression systems (native I-C CRISPR-Cas3 system and anti-anti-CRISPR proteins are not glycosylated). In certain embodiments, a variant of a I-C CRISPR-Cas3 system or anti-anti-CRISPR protein includes a glycosylation variant wherein the number and/or type of glycosylation sites have been altered relative to a naturally-occurring I-C CRISPR-Cas3 protein or anti-anti-CRISPR sequence expressed in a eukaryotic expression system.

crRNAs can be prepared, e.g., by chemical synthesis or by in vitro transcription, e.g., using a pUC19 or equivalent vector, e.g., containing a T7 transcription cassette, and purification of the produced crRNAs and, e.g., removal of the 5′ triphosphate group. RNPs, e.g., crRNA-protein complexes comprising the I-C CRISPR-Cas3 system components can be prepared by incubating the components together. Methods of chemically synthesizing RNA or of producing RNA in in vitro transcription systems are well known in the art.

The efficacy of crRNAs, I-C CRISPR-Cas3 systems, and RNPs can be assessed using any of a number of assays. For example, crRNAs and/or I-C CRISPR-Cas3 systems can be assessed using cell-based assays, e.g., in bacteria such as P. aeruginosa, Pseudomonas syringae, or E. coli, wherein the I-C CRISPR-Cas3 system is either endogenous or a heterologous system is introduced, and using crRNA directed, e.g., to a detectable marker such as phzM, lacZ, or to a phage wherein the efficacy of the crRNA and I-C CRISPR-Cas3 system is assessed by examining plaque formation. The generation of deletions, insertions, and genomic modifications can also be assessed by, e.g., delays or other alterations in the growth of cultured cells, as well as by standard molecular biology or biochemical methods for detecting deletions, insertions, or genomic modifications such as PCR, Sanger sequencing, whole genome sequencing, or Southern Blotting.

Delivery into Cells

Introduction of the I-C CRISPR-Cas3 system polynucleotides, polypeptides, RNPs, anti-anti-CRISPRs, or homologous repair templates into cells can take different forms. For example, in some embodiments, the polypeptides and/or RNPs themselves are introduced into the cells. Any method for the introduction of polypeptides or RNPs into cells can be used. For example, in some embodiments, electroporation, or liposomal or nanoparticle delivery to the cells can be employed. In other embodiments, one or more polynucleotides encoding a crRNA, anti-anti-CRISPR and/or one or more I-C CRISPR-Cas3 system polypeptides are introduced into the cell and the crRNA and/or I-C CRISPR-Cas3 system proteins are subsequently expressed in the cell. In some embodiments, the polynucleotide is an RNA molecule. In some embodiments, the polynucleotide is a DNA molecule.

In some embodiments, the crRNA, anti-anti-CRISPR and/or I-C CRISPR-Cas3 system proteins are expressed in the cell from RNA encoded by an expression cassette, wherein the expression cassette comprises a promoter operably linked to a polynucleotide encoding the crRNA, anti-anti-CRISPR and/or I-C CRISPR-Cas3 system proteins. In some embodiments, the promoter is heterologous to the polynucleotide encoding the crRNA, anti-anti-CRISPR and/or I-C CRISPR-Cas3 system proteins. Selection of the promoter will depend on the cell in which it is to be expressed and the desired expression pattern. In some embodiments, promoters are inducible or repressible, such that expression of a nucleic acid operably linked to the promoter can be expressed under selected conditions. In some examples, a promoter is an inducible promoter such as aTC, IPTG, or PBAD, such that expression of a nucleic acid operably linked to the promoter is activated or increased.

In embodiments where a polynucleotide is introduced that encodes an appropriate crRNA or polynucleotide encoding an I-C CRISPR-Cas3 component, e.g., Cas3, Cas5, Cas7, or Cas8, or an anti-anti-CRISPR, any suitable promoter can be used that will lead to a level of expression that is higher than the level in the absence of the construct. Any level of expression that is sufficient to induce deletions or gene repression in the cell can be used.

An inducible promoter may be activated by the presence or absence of a particular molecule, for example, doxycycline, tetracycline, metal ions, alcohol, or steroid compounds. In some embodiments, an inducible promoter is a promoter that is activated by environmental conditions, for example, light or temperature. In further examples, the promoter is a repressible promoter such that expression of a nucleic acid operably linked to the promoter can be reduced to low or undetectable levels, or eliminated. A repressible promoter may be repressed by direct binding of a repressor molecule (such as binding of the trp repressor to the trp operator in the presence of tryptophan). In a particular example, a repressible promoter is a tetracycline repressible promoter. In other examples, a repressible promoter is a promoter that is repressible by environmental conditions, such as hypoxia or exposure to metal ions.

In some embodiments, the polynucleotides encoding the crRNA, anti-anti-CRISPR and/or I-C CRISPR-Cas3 system proteins (e.g., as part of an expression cassette) are delivered to the cell by a vector. For example, in some embodiments, the vector is a viral vector. Exemplary viral vectors can include, but are not limited to, adenoviral vectors, adeno-associated viral (AAV) vectors, and lentiviral vectors.

Introduction of crRNA, anti-anti-CRISPR, homologous repair template, and/or I-C CRISPR-Cas3 system as described herein into a prokaryotic cell can be achieved by any method used to introduce protein or nuclei acids into a prokaryote. In some embodiments, the crRNA, anti-anti-CRISPR, homologous repair template and/or I-C CRISPR-Cas3 polypeptides are delivered to the prokaryotic cell by a delivery vector (e.g., a bacteriophage) that delivers a polynucleotide encoding the crRNA, anti-anti-CRISPR and/or one or more of the I-C CRISPR-Cas3 system polypeptide components.

In some embodiments, polynucleotides, e.g., homologous repair template, or polynucleotide encoding a crRNA, anti-anti-CRISPR and/or one or more I-C CRISPR-Cas3 components, are introduced into bacteria using phage, e.g., a phage delivery vector comprised of ssDNA or dsDNA that delivers DNA cargo to target cells. Any phage capable of introducing a polynucleotide into the target cell can be used. The phage could be, e.g., a tailed phage or a filamentous phage, that carries an entirely designed genome or that has heterologous genes introduced into an otherwise natural genome.

In other embodiments, polynucleotides, e.g., a homologous repair template, or polynucleotides encoding a crRNA, anti-anti-CRISPR, and/or one or more CRISPR-Cas3 component, are introduced into bacteria using bacterial conjugation. In some embodiments, polynucleotides are introduced into target prokaryotes using E. coli as a conjugative donor strain, e.g., using mobilizable plasmids that transfer their genetic material, e.g., polynucleotides encoding one or more crRNA and/or one or more I-C CRISPR-Cas3 component.

In certain embodiments, the crRNA, anti-anti-CRISPR, homologous repair template, and/or I-C CRISPR-Cas3 components are produced in vitro and introduced directly into cells, either individually or as a pre-formed RNP, i.e., a crRNA-Cas protein complex.

In certain embodiments, the crRNA, anti-anti-CRISPR, and/or I-C CRISPR-Cas3 system components are introduced into the cell by directly introducing RNA into the cell, e.g., the crRNA and/or mRNA encoding the I-C CRISPR-Cas system components.

In some embodiments, the crRNAs, anti-anti-CRISPR, and/or I-C CRISPR-Cas3 system components are introduced into cells using modified RNA. Various modifications of RNA are known in the art to enhance, e.g., the translation, potency and/or stability of RNA, e.g., crRNA or mRNA encoding a I-C CRISPR-Cas3 system component or anti-anti-CRISPR, when introduced into cells. In particular embodiments, modified mRNA (mmRNA) is used, e.g., mmRNA encoding a I-C CRISPR-Cas3 system component or anti-anti-CRISPR. In other embodiments, modified RNA comprising a crRNA is used. Non-limiting examples of RNA modifications that can be used include anti-reverse-cap analogs (ARCA), polyA tails of, e.g., 100-250 nucleotides in length, replacement of AU-rich sequences in the 3′UTR with sequences from known stable mRNAs, and the inclusion of modified nucleosides and structures such as pseudouridine, e.g., N1-methylpseudouridine, 2-thiouridine, 4′thioRNA, 5-methylcytidine, 6-methyladenosine, amide 3 linkages, thioate linkages, inosine, 2′-deoxyribonucleotides, 5-Bromo-uridine and 2′-O-methylated nucleosides. A non-limiting list of chemical modifications that can be used can be found, e.g., in the online database crdd.osdd.net/servers/sirnamod/. RNAs can be introduced into cells in vivo using any known method, including, inter alia, physical disturbance, the generation of RNA endocytosis by cationic carriers, electroporation, gene guns, ultrasound, nanoparticles, conjugates, or high-pressure injection. Modified RNA can also be introduced by direct injection, e.g., in citrate-buffered saline. RNA can also be delivered using self-assembled lipoplexes or polyplexes that are spontaneously generated by charge-to-charge interactions between negatively charged RNA and cationic lipids or polymers, such as lipoplexes, polyplexes, polycations and dendrimers. Polymers such as poly-L-lysine, polyamidoamine, and polyethyleneimine, chitosan, and poly(β-amino esters) can also be used. See, e.g., Youn et al. (2015) Expert Opin Biol Ther, September 2; 15 (9): 1337-1348; Kaczmarek et al. (2017) Genome Medicine 9:60; Gan et al. (2019) Nature comm. 10:871; Chien et al. (2015) Cold Spring Harb Perspect Med. 2015; 5: a014035; the entire disclosures of each of which are herein incorporated by reference.

In some embodiments, the crRNA, one or more I-C CRISPR-Cas3 system protein component, RNP, anti-anti-CRISPR, homologous repair template, or a polynucleotide encoding a crRNA, anti-anti-CRISPR and/or I-C CRISPR-Cas3 system protein is delivered as part of or within a cell delivery system. Various delivery systems are known and can be used to administer a composition of the present disclosure, for example, encapsulation in liposomes, microparticles, microcapsules, or receptor-mediated delivery.

Exemplary liposomal delivery methodologies are described in Metselaar et al., Mini Rev. Med. Chem. 2 (4): 319-29 (2002); O'Hagen et al., Expert Rev. Vaccines 2 (2): 269-83 (2003); O'Hagan, Curr. Drug Targets Infjct. Disord. 1 (3): 273-86 (2001); Zho et al., Biosci Rep. 22 (2): 355-69 (2002); Chikh et al., Biosci Rep. 22 (2): 339-53 (2002); Bungener et al., Biosci. Rep. 22 (2): 323-38 (2002); Park, Biosci Rep. 22 (2): 267-81 (2002); Ulrich, Biosci. Rep. 22 (2): 129-50; Lofthouse, Adv. Drug Deliv. Rev. 54 (6): 863-70 (2002); Zhou et al., J. Inmunmunother. 25 (4): 289-303 (2002); Singh et al., Pharm Res. 19 (6): 715-28 (2002); Wong et al., Curr. Med. Chem. 8 (9): 1123-36 (2001); and Zhou et al., Immunonmethods (3): 229-35 (1994).

Exemplary nanoparticle delivery methodologies, including gold, iron oxide, titanium, hydrogel, and calcium phosphate nanoparticle delivery methodologies, are described in Wagner and Bhaduri, Tissue Engineering 18 (1): 1-14 (2012) (describing inorganic nanoparticles); Ding et al., Mol Ther e-pub (2014) (describing gold nanoparticles); Zhang et al., Langmuir 30 (3): 839-45 (2014) (describing titanium dioxide nanoparticles); Xie et al., Curr Pharm Biotechnol 14 (10): 918-25 (2014) (describing biodegradable calcium phosphate nanoparticles); and Sizovs et al., J Am Chem Soc 136 (1): 234-40 (2014).

Introduction of an RNP, crRNA, anti-anti-CRISPR, homologous repair template and/or I-C CRISPR-Cas3 system protein as described herein into a prokaryotic cell can be achieved by any method used to introduce protein or nuclei acids into a prokaryote. In some embodiments, a crRNA, anti-anti-CRISPR, homologous repair template, and/or I-C CRISPR-Cas3 system protein or anti-anti-CRISPR is delivered to the prokaryotic cell by a delivery vector (e.g., a bacteriophage) that delivers a polynucleotide encoding the crRNA, anti-anti-CRISPR and/or I-C CRISPR-Cas3 system protein.

Exemplary cells that can be used in the present methods can be prokaryotic or eukaryotic cells. Exemplary prokaryotic cells can include but are not limited to, those used for biotechnological purposes, the production of desired metabolites, E. coli and human pathogens. Examples of such prokaryotic cells can include, for example, Escherichia coli, Pseudomonas sp., Corynebacterium sp., Bacillus subtitis, Streptococcus pneumonia, Pseudomonas aeruginosa, Staphylococcus aureus, Campylobacter jejuni, Francisella novicida, Corynebacterium diphtheria, Enterococcus sp., Listeria monocytogenes, Mycoplasma gallisepticum, Streptococcus sp., or Treponema denticola. In some embodiments, prokaryotic cells include pathogenic cells and/or antibiotic resistant cells. Exemplary eukaryotic cells can include, for example, fungal, animal (e.g., mammalian) or plant cells. Exemplary mammalian cells include but are not limited to human, non-human primates. mouse, and rat cells. Cells can be cultured cells or primary cells. Exemplary cell types can include, but are not limited to, induced pluripotent cells, stem cells or progenitor cells, and blood cells, including but not limited to hematopoietic stem cells, T-cells or B-cells.

In some embodiments, the cells are removed from an animal (e.g., a human, optionally in need of genetic repair, e.g., a genetic deletion, insertion, modification, or gene repression), and then a crRNA, homologous repair template, and/or I-C CRISPR-Cas3 system and/or anti-anti-CRISPR protein or polynucleotide, are introduced into the cell ex vivo. In some embodiments, the cell(s) is subsequently introduced into the same animal (autologous) or different animal (allogeneic).

In some embodiments, an RNP, crRNA, homologous repair template, and/or I-C CRISPR-Cas3 system protein as described herein can be introduced (e.g., administered) to an animal (e.g., a human) or plant or plant cell. This can be used to induce targeted deletions, insertions, genomic modifications, or gene repression in vivo, for example in situations in which I-C CRISPR-Cas3 mediated deletion, insertion, modification, or induction of gene repression or activation is performed in vivo.

In some embodiments, an RNP, crRNA, homologous repair template, anti-anti-CRISPR and/or I-C CRISPR-Cas3 system protein is administered as a pharmaceutical composition. In some embodiments, the composition comprises a delivery system such as a liposome, nanoparticle or other delivery vehicle as described herein or otherwise known, comprising the RNP, crRNA, homologous repair template, anti-anti-CRISPR, and/or I-C CRISPR-Cas3 system protein, or a polynucleotide encoding the crRNA, anti-anti-CRISPR and/or I-C CRISPR-Cas3 system protein. The compositions can be administered directly to a mammal (e.g., human) to induce targeted deletions, insertions, genomic modifications, or gene repression or activation using any route known in the art, including e.g., by injection (e.g., intravenous, intraperitoneal, subcutaneous, intramuscular, or intradermal), inhalation, transdermal application, rectal administration, or oral administration.

The pharmaceutical compositions may comprise a pharmaceutically acceptable carrier. Pharmaceutically acceptable carriers are determined in part by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

### Kits

Other embodiments of the compositions described herein are kits comprising a crRNA, I-C CRISPR-Cas3 system protein or proteins, homologous repair template, anti-anti-CRISPR, polynucleotide(s) encoding a crRNA of the invention and/or encoding a I-C CRISPR-Cas3 system protein or proteins or an anti-anti-CRISPR, and/or an RNP comprising a crRNA and one or more I-C CRISPR-Cas3 system protein. The kit typically contains containers, which may be formed from a variety of materials such as glass or plastic, and can include for example, bottles, vials, syringes, and test tubes. A label typically accompanies the kit, and includes any writing or recorded material, which may be electronic or computer readable form providing instructions or other information for use of the kit contents.

In some embodiments, the kits can further comprise instructional materials containing directions (i.e., protocols) for the practice of the methods of this invention (e.g., instructions for using the kit for inducing deletions, insertions, genomic modifications, and gene repression or activation in cells). While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD-ROM), and the like. Such media may include addresses to internet sites that provide such instructional materials.

### 5. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. a Minimal CRISPR-Cas3 System for Genome Engineering

### Abstract

CRISPR-Cas technologies have provided programmable gene editing tools that have revolutionized research. The leading CRISPR-Cas9 and Cas12a enzymes are ideal for programmed genetic manipulation, however, they are limited for genome-scale interventions. Here, we utilized a Cas3-based system featuring a processive nuclease for genome engineering purposes. This minimal CRISPR-Cas3 system (Type I-C), programmed with a single crRNA, was optimized to approach 100% efficiency, and used to rapidly generate large deletions ranging from 7-424 kb in Pseudomonas. By comparison, Cas9 yielded small deletions and point mutations. Cas3-generated deletion boundaries were variable, but successfully specified by a homology-directed repair (HDR) template. HDR was much more efficient when lesions were generated by Cas3, compared to Cas9. The minimal Cas3 system is also portable; using an “all-in-one” vector, large deletions could be efficiently generated in Pseudomonas syringae and Escherichia coli. Notably, Cas3 generated bi-directional deletions originating from the programmed cut site, which was exploited to rapidly and iteratively reduce a P. aeruginosa genome by 837 kb (13.5%) using 10 distinct crRNAs. We also enhance the utility of endogenous Cas3 systems by developing an “anti-anti-CRISPR” strategy to circumvent endogenous CRISPR-Cas inhibitor proteins. CRISPR-Cas3 could facilitate rapid strain manipulation for synthetic biological and metabolic engineering purposes, genome minimization, and the analysis of large regions of unknown function.

### Introduction

Here, we describe a repurposed Type I-C CRISPR system from Pseudomonas aeruginosa for genome engineering in microbes. Importantly, by targeting the genome with a single crRNA and selecting only for survival after editing, this tool is a counter-selection-free approach to programmable genome editing. CRISPR-Cas3 is capable of efficient genome-scale modifications currently not achievable using other methodologies. It has the potential to serve as a powerful tool for basic research, discovery, and strain optimization.

### Results

Implementation and Optimization of Genome Editing with CRISPR-Cas3

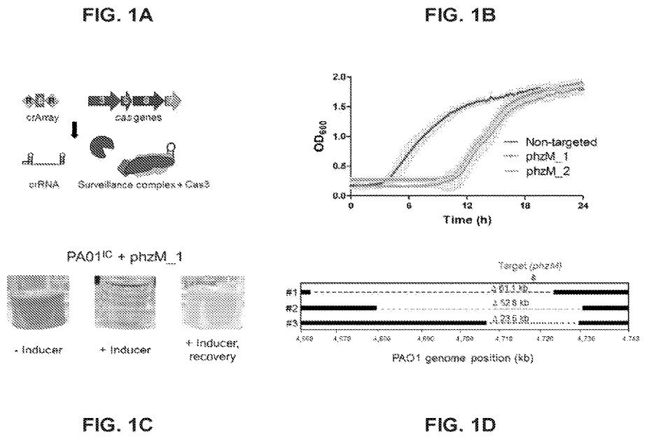

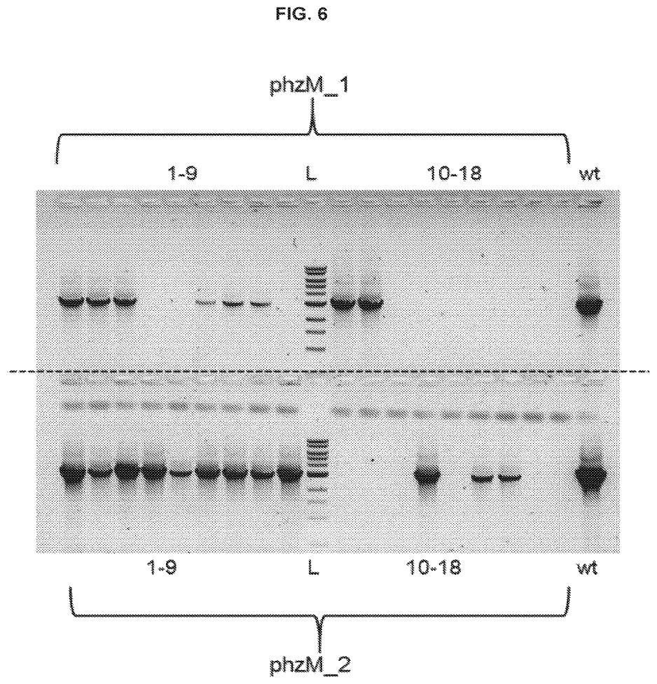

Type I-C CRISPR-Cas systems utilize just three cas genes (cas5, cas8, and cas7) to produce the crRNA-guided Cascade surveillance complex that can recruit Cas3 (FIG. 1A), making it a minimal system (34, 35). A previously constructed (36) Pseudomonas aeruginosa PAO1 strain (PAO1^{IC}) with inducible cas genes and crRNAs (26) was used here to conduct targeted genome manipulation. The expression of a crRNA targeting the genome caused a transient growth delay (FIG. 1B), but survivors were isolated after extended growth. By targeting phzM, a gene required for production of a blue-green pigment (pyocyanin), we observed yellow cultures (FIG. 1C) for 16 out of 36 (44%) biological replicates (18 recovered isolates from two independent phzM-targeting crRNAs). PCR of genomic DNA confirmed that the yellow cultures had lost this region, while blue-green survivors maintained it (FIG. 6). Three of these deletion strains were sequenced, revealing deletions of 23.5 kb, 52.8 kb, and 60.1 kb, and each one was bi-directional relative to the crRNA target site (FIG. 1D). This demonstrated the potential for Type I-C Cas3 systems to be used to induce large genomic deletions with random boundaries surrounding a programmed target site.

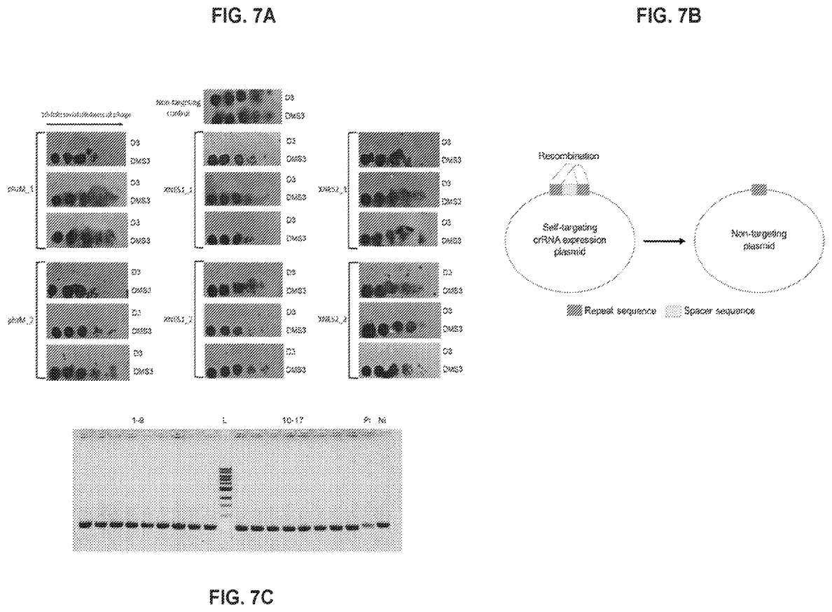

To determine the in vivo processivity of the Cas3 enzyme, we targeted 2 of the 16 extended non-essential (XNES) regions>100 kb in length (Table 1) identified from a transposon sequencing (TnSeq) data set (27). The frequency of deletions generated by crRNAs targeting XNES 1 and XNES 2 (along with additional targeting of phzM, which is found in XNES 15) was quantified, revealing that 20-40% of the surviving colonies had deletions (FIG. 2A). To understand how cells lacking large deletions had survived self-targeting, three possibilities were considered: i) a cas gene mutation, ii) a PAM or protospacer mutation, or iii) a mutation to the plasmid expressing the crRNA. Three survivors lacking target deletions from each of the six self-targeting crRNAs were assayed. All had functional cas genes when the self-targeting crRNA was replaced with a phage-targeting crRNA (FIG. 7A), and target sequencing revealed no point mutations. PCR-amplification and sequencing of the crRNA-expressing plasmids isolated from the survivors revealed the primary escape mechanism: recombination between the direct repeats, leading to the loss of the spacer (FIG. 7B). An additional 17 survivors that lacked deletions were assayed via PCR and were also ~60 bp shorter (FIG. 7C), consistent with the loss of one repeat and spacer.

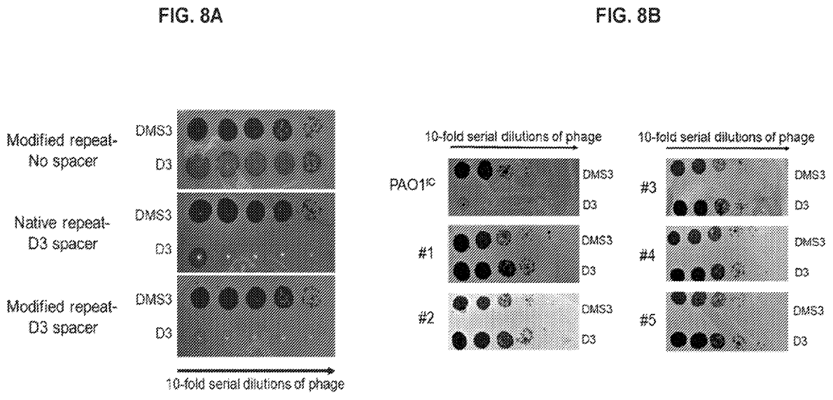

Spacer excision was successfully prevented by engineering a modified repeat (MR), with six mutated nucleotides in the stem and three in the loop of the second repeat (FIG. 2B), disrupting homology between the two direct repeats. A phage-targeting crRNA with this new design targeted phage as well as or better than the same crRNA with unmodified repeats (FIG. 8A). Using the same self-targeting spacers designed against phzM, XNES 1, and XNES 2 with the MR resulted in a robust increase in editing efficiencies to 94-100% for the six tested crRNAs (FIG. 2A) and spacer excision was no longer detected. 211 of 216 (98%) total survivor cells had large deletions based on PCR screening (i.e., >1 kb), while the remaining 5 had inactive CRISPR-Cas systems when tested with the phage-targeting crRNA (FIG. 8B).

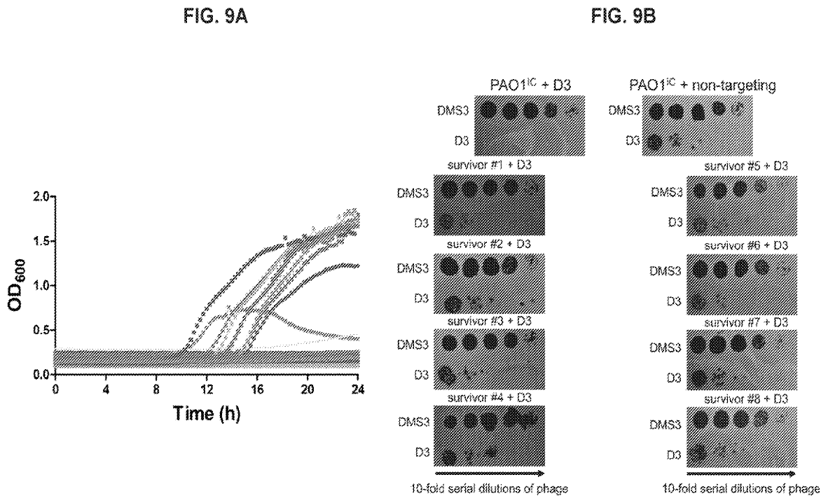

The processivity of Cas3 could likely lead to unintended deletions of neighboring essential genes, if targeting is initiated nearby. To assess the phenotype of such an event, we intentionally targeted an essential gene, rplQ (a 50S ribosomal subunit protein) (38). Two different MR crRNAs targeting rplQ led to a severely extended lag time compared to non-essential gene targeting. Only 8 out of 36 rplQ-targeting biological replicates grew after 24 hours, compared to the transient growth delay of ~12 hours when targeting non-essential genes (FIG. 9A). Subsequent analysis of these 8 survivor cultures with phage targeting assays revealed non-functional cas genes (FIG. 9B). Importantly, no spacer excision events were detected in this experiment or among the 216 replicates screened above. This experiment highlights the robustness of the deletion method, as the outcome of essential gene versus non-essential gene targeting is noticeably distinct.

Cas3 Generates Larger Deletions than Cas9 and is More Recombinogenic

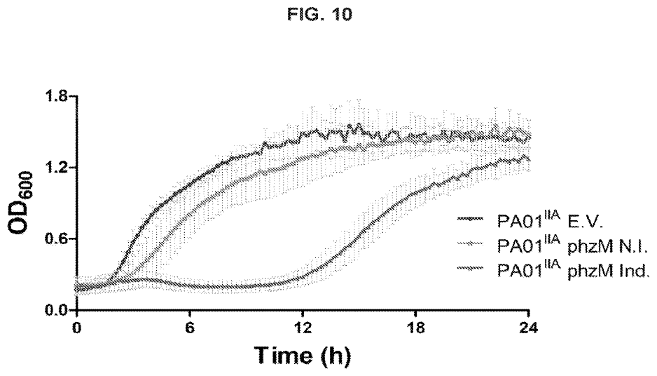

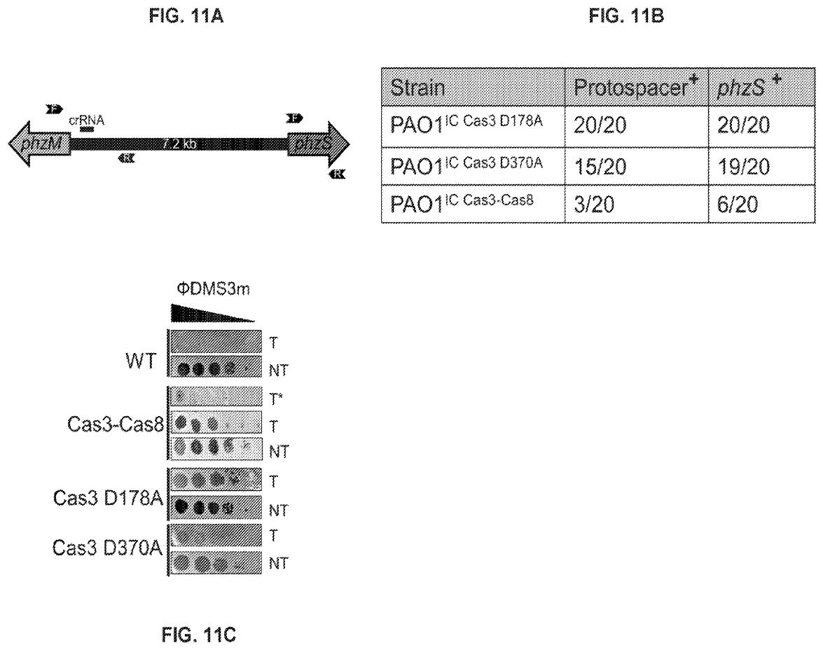

To determine whether large deletions are a direct consequence of the Cas3 enzyme and its processivity, we compared self-targeting outcomes to an isogenic strain expressing the non-processive Streptococcus pyogenes Cas9 (PAO1″IA) and to a helicase-deficient Cas3 mutant. Two Cas9 sgRNAs that recognized sites overlapping with the crRNAs used for Cas3 were targeted to phzM (FIG. 2E, FIG. 10). PCR and sequencing analysis of these surviving cells revealed that deletions larger than 1 kb were a rare occurrence (5.6% assayed survivor cells, n=72) compared to 98.6% with Cas3 (FIG. 2E). Whole-genome sequencing (WGS) of two large deletion survivors selected for by Cas9 showed lesions of 5 kb and 23 kb around the target site, respectively. The more common modes of survival after Cas9 targeting were small deletions between 0.1-0.5 kb in length (25% of all survivors), or 1-3 bp protospacer/PAM deletions/mutations (19.4%). Similarly, the helicase inactive Cas3 variant (Cas3 D370A) generated smaller deletions than its wild-type counterpart, and had a lower efficiency (~25% of survivors were edited cells) (FIGS. 11A-11B). As expected, a nuclease deficient mutant (Cas3 D178A) did not generate any detectable mutations (FIG. 11B).

As a final mechanism to probe Cas3 processivity, and in an effort to further minimize the system, Cas3 was covalently tethered to the Cas8-Cas5-Cas7 complex via fusion with Cas8. This was motivated by similar fusions in nature and previous experimental work in the Type I-E system (6). This fusion was active, still displaying partial phage targeting immunity (FIG. 11C) and yielding 85% deletion efficiency (17/20). However, 6 of the 17 survivors assayed maintained a gene located 7.2 kb away (FIGS. 11A-11B). This smaller deletion distribution is consistent with previous single molecule work that revealed Cas3 translocating in association with the Cascade complex for ~10-20 kb, before Cascade “snaps back” to its starting point and Cas3 continues (39). Tethering Cas3 to the Cascade complex likely limits Cas3 processivity. In sum, the shift of deletions toward smaller size resulting from targeting with the non-processive SpyCas9, a non-processive Cas3 helicase mutant, or a modestly processive tethered Cas3, directly implicates Cas3's enzymatic activity as the cause of large deletions.

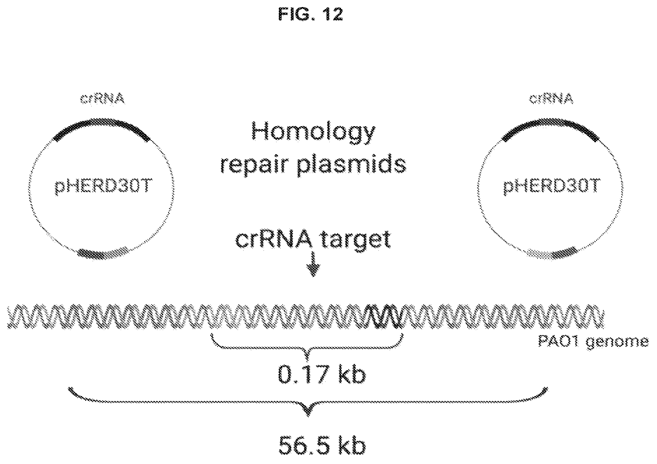

The direct relationship between Cas3 nuclease-helicase activity and survival via large deletions led us to hypothesize that its processive ssDNA nuclease activity may promote recombination by exposing regions of ssDNA. To test this, we provided a repair template with 500 bp of the upstream and downstream regions flanking the desired deletion to enable homology directed repair (HDR). We chose 0.17 kb and 56.5 kb deletions around phzM and a 249 kb deletion within XNES8 for the programmed deletions (FIG. 12). The recombination efficiencies were significantly higher with Cas3 than with Cas9 (FIG. 2F). The 249 kb deletion was incorporated in 22% of the Cas3-generated survivors, compared to 0% using Cas9 (χ² (1, N=72)=9, p=2.7E-03). The 56.5 kb deletion had an efficiency of 61% vs. 5.5% (χ² (1, N=72)=25, p=5.73E-07), and the 0.17 kb deletion had an efficiency of 100% vs. 39% when targeting with Cas3 or Cas9, respectively (χ² (1, N=72)=31.68, p=1.82E-08). These data support the hypothesis that Cas3 enhances recombination at cleavage sites and can be efficiently used for precisely programmed large genomic deletions.

Rapid Genome Minimization of P. aeruginosa Using CRISPR-Cas3 Editing

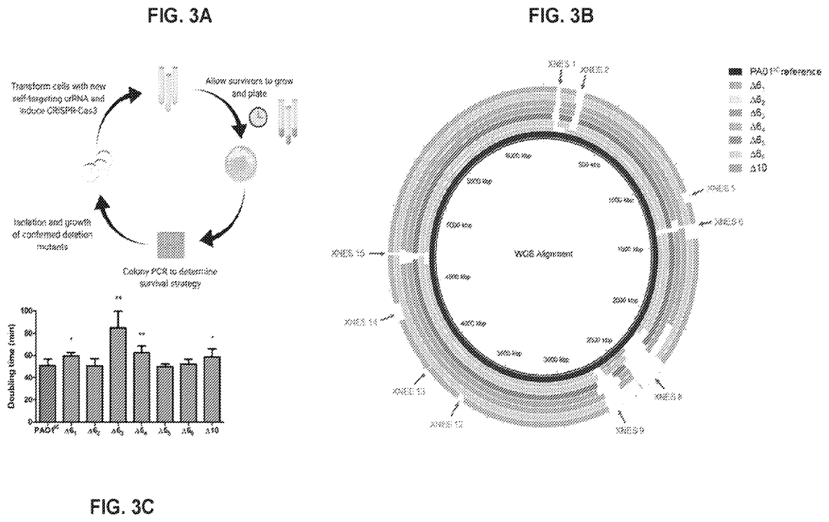

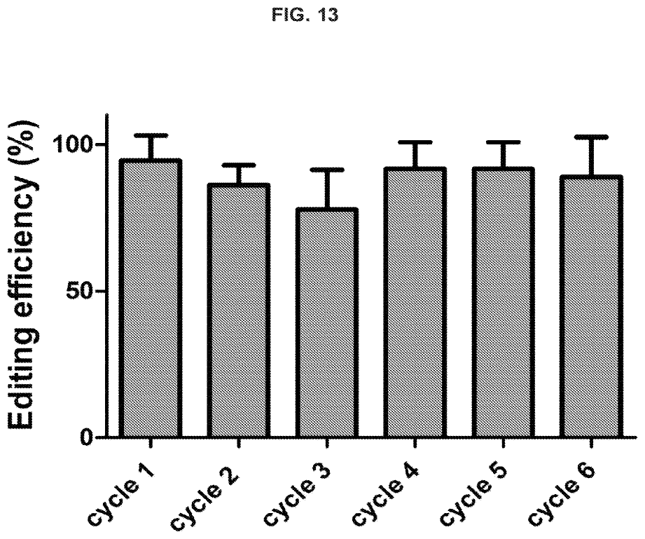

Large deletions with undefined boundaries provide an unbiased mechanism for genome streamlining, screening, and functional genomics. To demonstrate the potential for Cas3, we aimed to minimize the genome of P. aeruginosa through a series of iterative deletions of the XNES regions (FIG. 3A). Six XNES regions (including XNES 15, carrying phzM) were iteratively targeted in six parallel lineages (FIG. 3B), resulting in 35 independent deletions (WGS revealed no deletion at XNES 2 in one of the strains). Deletion efficiency remained high (>80%) throughout each round of self-targeting (FIG. 13). WGS of these 6 multiple deletion strains (Δ61-Δ66) revealed that no two deletions had the exact same coordinates, highlighting the stochastic nature of Cas3. The smallest isolated deletion was 7 kb and the largest 424 kb (mean: 92.9 kb, median: 58.2 kb). Of note, 4 genes (PA0123, PA1969, PA2024, and PA2156) previously identified as essential (37) were deleted in at least one of the lineages. Most deletions appeared to be resolved by flanking microhomology regions (Table 2), implicating alternative-end joining (40) as the dominant repair process.

To minimize the genome further, one of the already reduced strains was subjected to 4 additional rounds of deletions at XNES regions for a total of 10 genomic deletions (Δ10, FIG. 3B). Whole-genome sequencing of the 410 strain showed a genome reduction of 849 kb (13.6% of the genome). Generation of large deletions resulted in a growth defect in some cases, with significantly slower growth in 3 of the 6 deletions strains (Δ6₁, Δ6₃, and Δ6₄), with the other 3 growing normally (FIG. 3C). Δ10 also displayed a slight decrease in fitness, showing a ~15% increase in doubling time compared to the parent strain. The general subtlety of the growth defects was likely bolstered by the selection of fast-growing colonies at each deletion round.

### CRISPR-Cas3 Editing in Distinct Bacteria

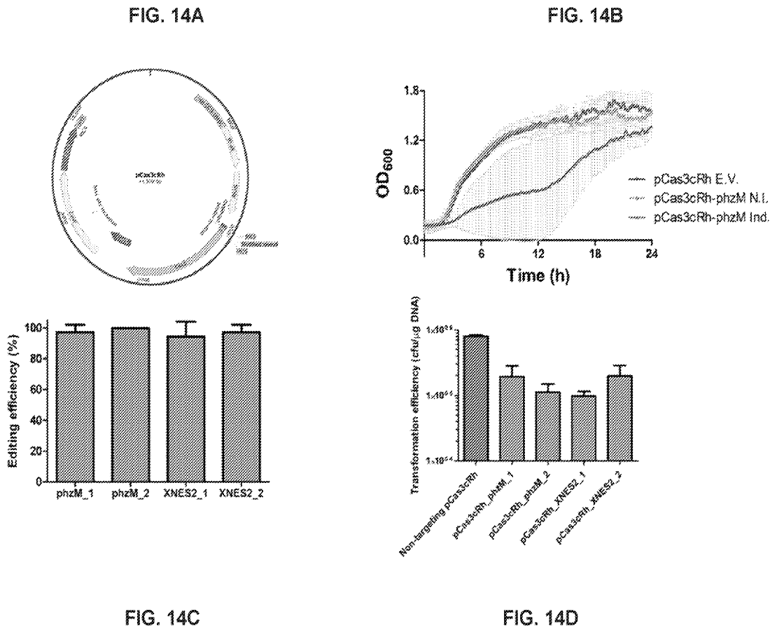

To enable expression of this system in other hosts, we constructed an all-in-one vector (pCas3cRh) carrying the I-C specific crRNA with a modified repeat sequence, cas3, cas5, cas8, and cas7 (FIG. 14A). As a pilot experiment, we transformed wild-type PAO1 with a non-targeting crRNA and crRNAs targeting phzM and XNES2. Induction of the targeting crRNAs induced editing efficiencies between 95-100% (FIGS. 14B-D).

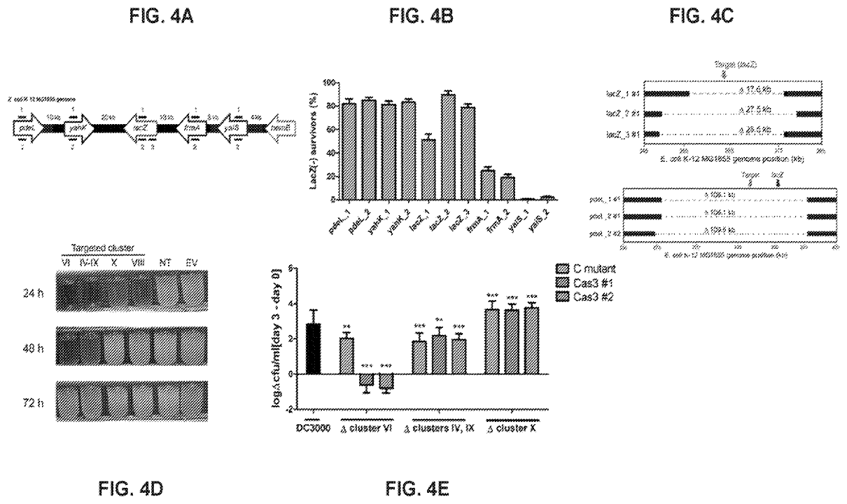

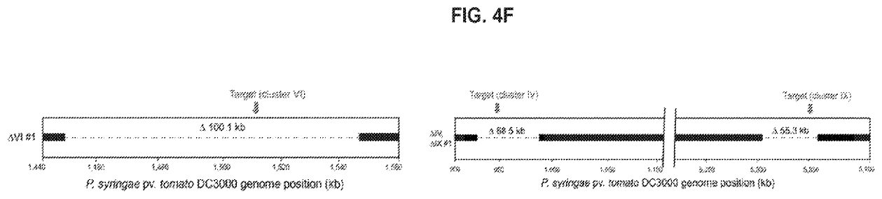

Having verified that pCas3cRh was functional, we tested this system in the model organism Escherichia coli K-12 MG1655. crRNAs were designed to target lacZ or its vicinity (FIG. 4A), where it is flanked by non-essential DNA (124.5 kb upstream, 22.4 kb downstream). Transformations were plated directly on inducing media containing X-gal and scored using blue/white screening. Depending on the crRNA used, directly targeting lacZ or 30 kb upstream yielded 51-90% or 82-85% editing efficiencies, respectively (FIG. 4B). 95 of the 96 LacZ (−) survivors assayed by PCR showed an absence of the lacZ region. crRNAs downstream of lacZ, however, had reduced efficiency as they approached the essential gene, hemB. frmA targeting (13 kb downstream of lacZ) had lower editing efficiencies (21-25%) and yaiS (18 kb downstream of lacZ) even lower (2%). This decrease in efficiency was independent of the strand being targeted (and therefore the predicted strand for Cas3 loading and 3′-5′ translocation), confirming the importance of Cas3 bi-directional deletions. Indeed, WGS of selected AlacZ cells revealed bi-directional deletions ranging from 17.5-106 kb encompassing the targeted region (FIG. 4C).

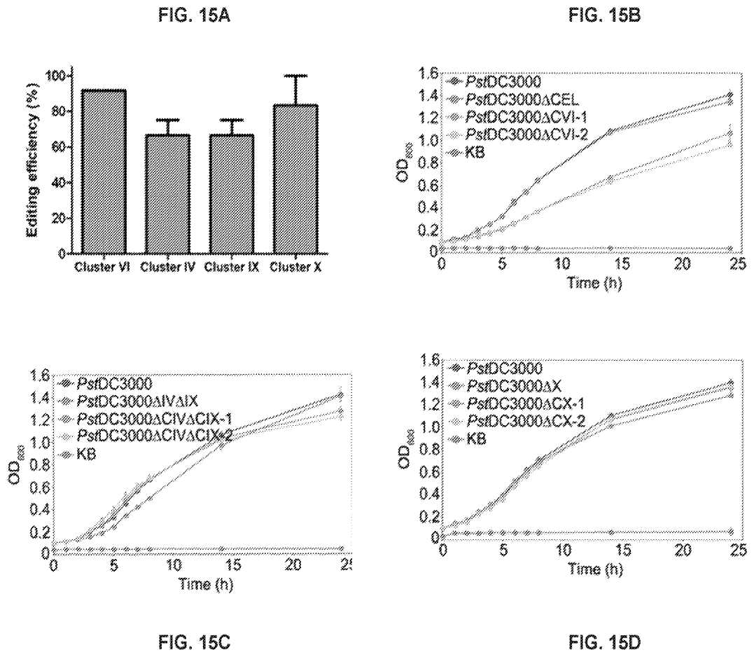

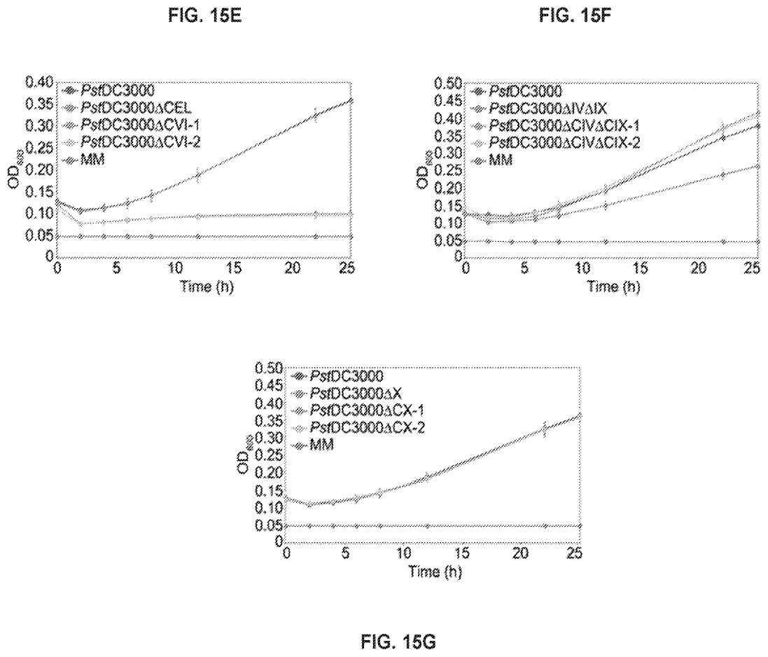

Next, we tested Cas3-mediated editing in the plant pathogen Pseudomonas syringae pv. tomato DC3000, which does not naturally encode a CRISPR-Cas system (41). P. syringae encodes many non-essential virulence effector genes whose activities are difficult to disentangle due to their redundancy (42). We designed crRNAs targeting four chromosomal virulence effector clusters (IV, VI, VIII, and IX), or one plasmid cluster (pDC3000 (43), cluster X) in P. syringae strain DC3000. Two clusters (IV and IX) shared identical sequences that could be targeted simultaneously using a single crRNA. Expression of targeting crRNAs led to a noticeable growth delay compared to non-targeted controls (FIG. 4D). PCR analysis of surviving cells showed editing efficiencies of 67-92% (FIG. 15A). In planta and in vitro growth assays of three deletion mutants effectively recapitulated the phenotypes of previously described cluster deletion polymutants (43) (FIG. 4E, FIGS. 15B-G). Targeting cluster X cured the 73 kb plasmid and simultaneous cluster IV and IX targeting led to dual deletions in 8 out of 12 survivors, with a sequenced representative having 68.5 kb and 55.3 kb deletions, respectively, at the expected target sites. The effector cluster VI Cas3-derived mutant had a more severe growth defect in vitro and in planta than the control mutant (FIG. 4E, FIGS. 15B, 15E). This large deletion (100.1 kb in size) likely impacted general fitness (FIG. 4F), demonstrating one drawback of large deletions, but this can be easily overcome by assessing in vitro growth of >1 mutant generated by each crRNA. Using our portable minimal system, we achieved three new applications: the single-step deletion of large virulence regions, multiplexed targeting, and plasmid curing. Overall, we have demonstrated I-C CRISPR-Cas3 editing to be a generally applicable tool capable of generating large genomic deletions in three distinct bacteria.

### Repurposing Endogenous CRISPR-Cas3 Systems for Gene Editing

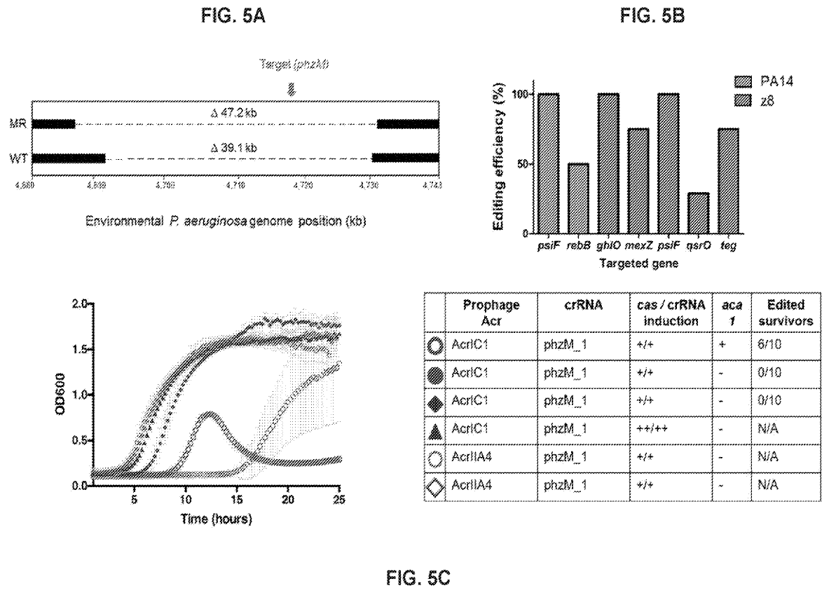

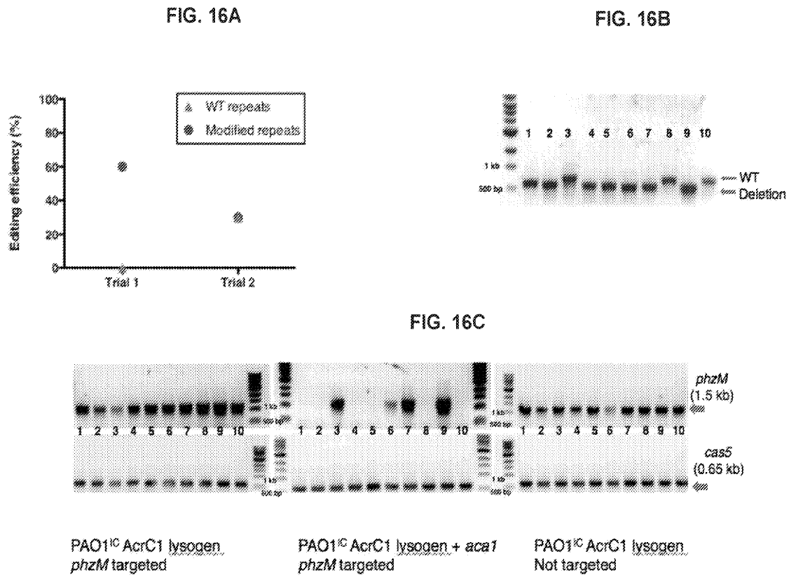

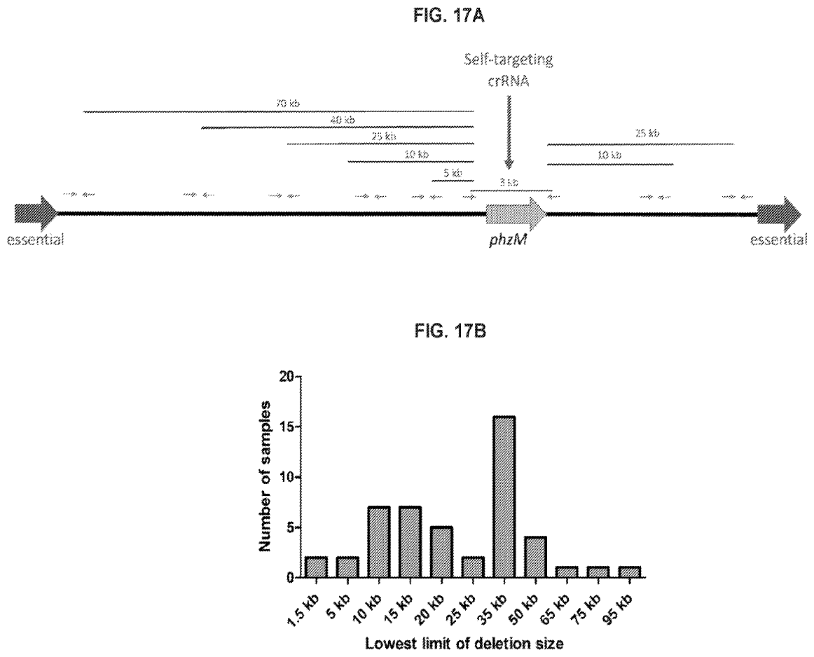

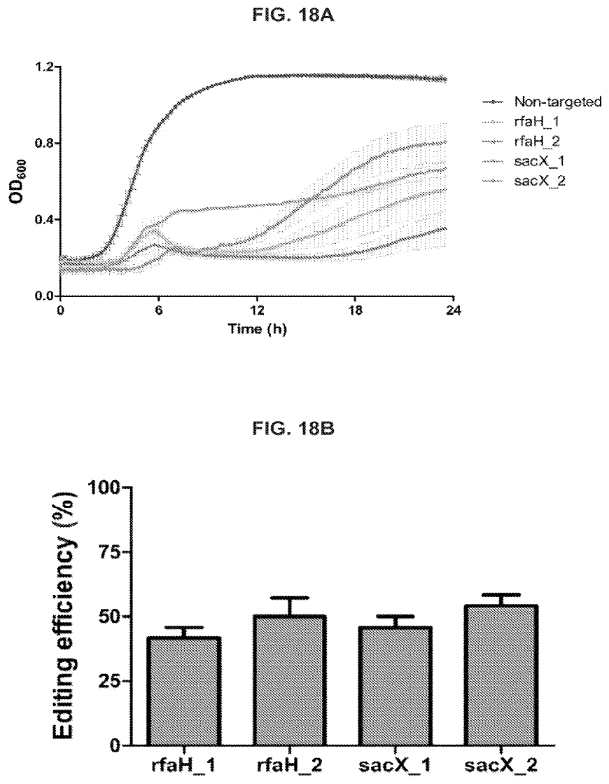

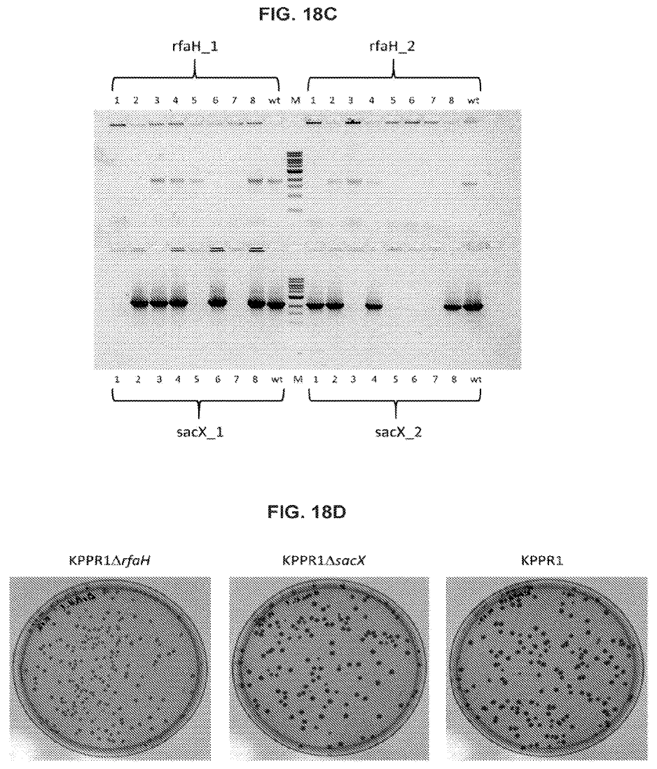

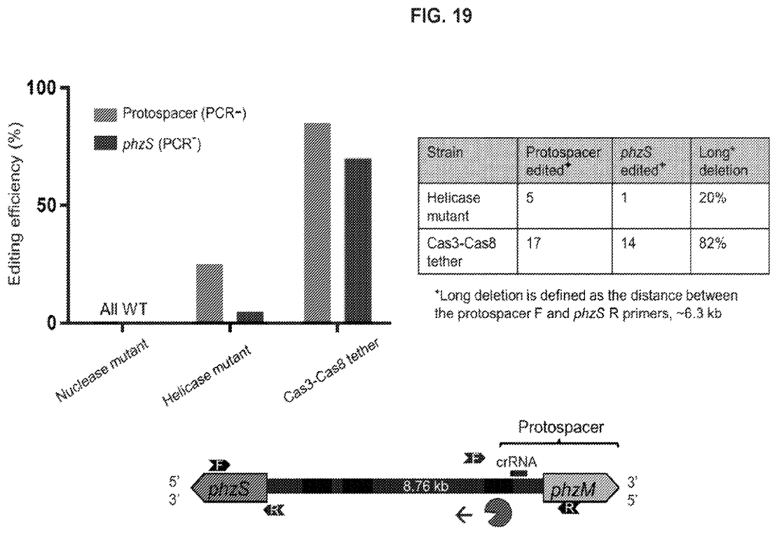

Type I CRISPR-Cas3 systems are the most common CRISPR-Cas systems in nature (1). Therefore, many bacteria have a built-in genome editing tool to be harnessed. We first tested the environmental isolate from which our Type I-C system was derived. Self-targeting phzM crRNAs led to the isolation of genomic deletions (FIG. 16A), with WGS revealing 33.7 (wild-type repeat) and 39 kb (MR) deletions of the target gene and surrounding regions (FIG. 5A). Additionally, HDR-based editing with a single construct was again efficacious, with 7/10 survivors acquiring the specific 0.17 kb deletion (FIG. 16B).

We next evaluated the feasibility of repurposing other Type I systems, using the naturally active Type I-F systems (44) encoded by laboratory strain P. aeruginosa PA14, and the clinical strain P. aeruginosa z8. Plasmids with Type I-F specific crRNAs were expressed, targeting various genomic sites for deletion (Table 3). HDR templates (600 bp arms on average) were included in the plasmids to generate deletions of defined coordinates ranging from 0.2 to 6.3 kb. Overall, at 5 different genomic target sites in strain z8 and 2 sites in PA14, we observed desired deletions in 29-100% of analyzed survivor colonies (FIG. 5B). Together, these experiments demonstrate the capacity for different forms of high efficiency genome editing using a single plasmid and an endogenous CRISPR-Cas system.

Finally, one potential impediment to the implementation of any CRISPR-Cas bacterial genome editing tool is the presence of anti-CRISPR (acr) proteins that inactivate CRISPR-Cas activity (45). In the presence of a prophage expressing AcrIC1 (a Type I-C anti-CRISPR protein) (36) from a native acr promoter, self-targeting was completely inhibited, but not by an isogenic prophage expressing a Cas9 inhibitor AcrIIA4 (45) (FIG. 5C). To attempt to overcome this impediment, we expressed aca1 (anti-CRISPR associated gene 1), a direct negative regulator of acr promoters (47), from the same construct as the crRNA. Using this repression-based “anti-anti-CRISPR” strategy, CRISPR-Cas function was re-activated, allowing the isolation of edited cells despite the presence of acrIC1 (FIGS. 5C and 16C). In contrast, simply increasing cas gene and crRNA expression did not overcome AcrIC1-mediated inhibition (FIG. 5C). Therefore, using anti-CRISPR repressors presents a viable route towards enhanced efficiency of CRISPR-Cas editing and necessitates continued discovery and characterization of anti-CRISPR proteins and their cognate repressors.

### Discussion

By repurposing a minimal CRISPR-Cas3 system as both an endogenous and heterologous genome editing tool, we show that hurdles to generating large deletions can be overcome. We obtained high efficiencies after modifying a repeat sequence to prevent spacer loss. Using only a single crRNA, we isolated deletions as large as 424 kb without requiring the insertion of a selectable marker or HDR templates guiding the repair process. Additionally, the I-C system appears to produce bi-directional deletions, similar to what was previously observed with the I-F CRISPR-Cas3 system (48), but not with type I-E (10, 11, 30). CRISPR-Cas3 presents a genome editing tool useful for the targeted removal of large elements (e.g. virulence clusters, plasmids) (49) and also for unbiased screening and genome streamlining. As a long-term goal of microbial gene editing has been genome minimization (50, 51, 57), we used our optimized CRISPR-Cas3 system to generate ten iterative deletions, achieving >13% genome reduction of the targeted strain. This spanned only 30 days while maintaining editing efficiency, a great improvement over previous genome reduction methods (52). We are currently extending deletions to all 16 XNES regions. Some basic microbial applications of Cas3 include studying chromosome biology (e.g. replichore asymmetry) (53), virulence factors (54), and the impact of the mobilome.

An important outcome of this work is the enhanced recombination observed at cut sites when comparing Cas3 and Cas9 directly. The potential for Cas3 to be more recombinogenic through the generation of exposed ssDNA may be advantageous for both programmed knock-outs and knock-ins. The direct comparison presented here between Cas3 (large deletions) and Cas9 (small deletions), and Cas3 variants also confirms the causality of Cas3 in the deletion outcomes.

Our study has revealed some of the benefits and challenges of working with CRISPR-Cas3. While some of the iteratively edited strains demonstrated slight growth defects, the Cas3 editing workflow shows high potential for genome minimization efforts. Since many distinct deletion events are generated, screening various isolates for fitness benefits or defects is possible, and one can proceed with the strain that has the desired fitness property. Despite our success at transplanting the minimal Type I-C system, it remains to be seen whether the approach will be limited by differences in DNA repair mechanisms. Indeed, in E. coli and P. syringae, larger regions of homology, such as 34 bp long REP sequences were observed (55), indicating the role of RecA-mediated homologous recombination (56) in the repair process. Meanwhile in P. aeruginosa, the borders of the deletions showed either small (4-14 bp) micro-homology or no noticeable sequence homology. The former implies a role for alternative end-joining (40), while the latter non-homologous end-joining (57) in the repair process. Efforts are underway to test this all-in-one system in Legionella pneumophila and Klebsiella pneumoniae to expand its utility. Downstream studies are required to dissect the roles of each mechanism in the deletion generation process for better predictable deletion outcomes.

CRISPR-Cas3 is an especially promising tool for use in eukaryotic cells as it would facilitate the interrogation of large segments of non-coding DNA, much of which has unknown function (58). Additionally, it was recently shown that Cas9-generated “gene knockouts” (i.e., small indels causing out-of-frame mutations) frequently encode pseudo-mRNAs that may produce protein products, necessitating methods for full gene removal (59, 60). Encouragingly, Type I-E CRISPR-Cas systems were recently shown to generate large deletions in human cells (30-32), demonstrating the potential wide applicability of Cas3. Overall, the intrinsic properties of Cas3 make it a promising tool to fill a void in current gene editing capabilities. Employing Cas3 to make large genomic deletions will facilitate the manipulation of repetitive and non-coding regions, having a broad impact on genetics research by providing a tool to probe genomes en masse.

### Methods

### Bacterial Strains, Plasmids, DNA Oligonucleotides, and Media

A previously described (36) environmental strain of Pseudomonas aeruginosa was used as a template to amplify the four cas genes of the Type I-C CRISPR-Cas system genes (cas3, cas5, cas7, and cas8). The genes were cloned into the pUC18-mini-Tn7T-LAC vector (61) using the SacI-PstI restriction endonuclease cut sites in the order cas5, cas7, cas8, cas3 to generate the plasmid pJW31 (Addgene number: 136423). This vector was introduced into Pseudomonas aeruginosa PAO1 (62), inserting the cas genes into the chromosome, following previously described methods (63). Following integration, the excess sequences, including the antibiotic resistance marker, were removed via Flp-mediated excision as described previously (63). The resulting strain, dubbed PAO1^{IC}, allowed for inducible expression of the I-C system through induction with isopropyl β-D-1 thiogalactopyranoside (IPTG). This same method was used to integrate the Cas3-Cas8 tether mutant in the order cas5, cas3, cas8, cas7. The linker amino acid sequence is RSTNRAKGLEAVS. An isogenic strain carrying Cas9 derived from Streptococcus pyogenes was constructed in the same fashion, resulting in the strain PAO1^{IIA}. For experiments to test the system in Pseudomonas syringae, we employed the previously characterized strain DC3000 (41). E. coli editing experiments were conducted with strain K-12 MG1655 (64).

To construct the Cas3 helicase and nuclease mutant strains, the PAO1IC system was utilized to introduce point mutations. crRNAs were designed to target Cas3 along with a homology directed repair (HDR) template that included the desired mutation, and silent mutations to prevent CRISPR-Cas targeting of the final strain.

To achieve genomic self-targeting of the I-C CRISPR-Cas strains, crRNAs designed to target the genome were expressed from the pHERD20T and pHERD30T shuttle vectors (65). So-called “entry vectors” pHERD20T-ICcr and pHERD30T-ICcr were first generated by cloning at the EcoRI and HindIII sites an annealed linear dsDNA template carrying the I-C CRISPR-Cas system repeat sequences flanking two BsaI Type IIS restriction endonuclease recognition sites. Additionally, a preexisting BsaI site in a non-coding site of the pHERD30T and pHERD20T plasmids was mutated using whole-plasmid amplification so it would not interfere with the cloning of the crRNAs (36). Oligonucleotides with repeat-specific overhangs encoding the various spacer sequences were annealed and phosphorylated using T4 polynucleotide kinase (PNK) and cloned into the entry vectors using the BsaI sites. For experiments using Cas9, sgRNAs were expressed from the same pHERD30T vector, with the sgRNA construct cloned using the same restriction sites as with the I-C crRNAs.

The all-in-one vector pCas3cRh (Addgene number 133773) is a derivative of the pHERD30T-IC plasmid, with the 4 I-C system genes cloned downstream of the crRNA site. This was achieved by amplifying the genes cas3, cas5, cas8, and cas7 in two fragments with a junction within cas8 designed to eliminate an intrinsic BsaI site with a synonymous point mutation. The amplified fragments were cloned into pHERD30T-IC using the Gibson assembly protocol (66). Finally, to guard against potential leaky toxic expression, we replaced the araC-ParaBAD promoter with the rhamnose-inducible rhaSR-PrhaBAD system (67). The sequence for rhaSR-PrhaBAD was amplified from the pJM230 template (67), and cloned into the pHERD30T-IC plasmid to replace araC-ParaBAD using Gibson Assembly (New England Biolabs). Without induction, transformation efficiencies of targeting constructs of assembled pCas3cRh were on average 5-10-fold lower when compared to non-targeting controls (FIG. 13C), indicating residual leakiness of the I-C system.

The aca1-containing vector pICcr-aca1 is a derivative of the pHERD30T-ICcr plasmid, with aca1 cloned downstream of the crRNA site under the control of the pBAD promoter. The aca1 gene was cloned from P. aeruginosa phage DMS3m.

All oligonucleotides used in this study were obtained from Integrated DNA Technologies. For a complete list of all DNA oligonucleotides and a short description, see Table 4.

P. aeruginosa and E. coli strains were grown in standard Lysogeny Broth (LB): 10 g tryptone, 5 g yeast extract, and 10 g NaCl per 1 L dH₂O. Solid plates were supplemented with 1.5% agar. P. syringae was grown in King's medium B (KB): 20 g Bacto Proteose Peptone No. 3, 1.5 g K₂HPO₄, 1.5 g MgSO₄·7H₂O, 10 ml glycerol per 1 L dH₂O, supplemented with 100 μg/ml rifampicin. The following antibiotic concentrations were used for selection: 50 μg/ml gentamicin for P. aeruginosa and P. syringae, 15 μg/ml for E. coli; 50 μg/ml carbenicillin for all organisms. Inducer concentrations were 0.5 mM IPTG, 0.1% arabinose, and 0.1% rhamnose. For transformation protocols, all bacteria were recovered in Super optimal broth with catabolite repression (SOC): 20 g tryptone, 5 g yeast extract, 10 mM NaCl, 2.5 mM KCl, 10 mM MgCl₂, 10 mM MgSO₄, and 20 mM glucose in 1 L dH₂O.

### Bacterial Transformations

Transformations of P. aeruginosa, E. coli, and P. syringae strains were conducted using standard electroporation protocols. 10 ml of overnight cultures were centrifuged and washed twice in an equal volume of 300 mM sucrose (20% glycerol for E. coli) and suspended in 1 ml 300 mM sucrose (20% glycerol for E. coli). 100 μl aliquots of the resulting competent cells were electroporated using a Gene Pulser Xcell Electroporation System (Bio-Rad) with 50-200 ng plasmid with the following settings: 200 Ω, 25 μF, 1.8 kV, using 0.2 mm gap width electroporation cuvettes (Bio-Rad). Electroporated cells were incubated in antibiotic-free SOC media for 1 hour at 37° C. (28° C. for P. syringae), then plated onto LB agar (KB agar for P. syringae) with the selecting antibiotic, and grown overnight at 37° C. (28° C. for P. syringae). Cloning procedures were performed in commercial E. coli DH5a cells (New England Biolabs) or E. coli XL1-Blue (QB3 Macrolab Berkeley), according to the manufacturer's protocols.

### Construction of Recombinant DMS3m Acr Phages

The isogenic DMS3m acrIIA4 and acrIC1 phages were constructed using previously described methods (68). A recombination cassette, pJZ01, was constructed with homology to the DMS3m acr locus. Using Gibson Assembly (New England Biolabs), either acrIC1 or acrIIA4 were cloned upstream of aca1, and the resulting vectors were used to transform PAO1^{IC}. The transformed strains were infected with WT DMS3m, and recombinant phages were screened for. Phages were stored in SM buffer at 4° C.

### Isolation of PAO1^{IC} Lysogens

PAO1^{IC} was grown overnight at 37° C. in LB media. 150 μl of overnight culture was added to 4 ml of 0.7% LB top agar and spread on 1.5% LB agar plates supplemented with 10 mM MgSO4. 5 μl of phage, expressing either acrIC1 or acrIIA4 were spotted on the solidified top agar and plates were incubated at 30° C. overnight. Following incubation, bacterial growth within the plaque was isolated and spread on 1.5% LB agar plate. After an overnight incubation at 37° C., single colonies were assayed for the prophage. Confirmed lysogens were used for genomic targeting experiments.

### Genomic Targeting

Pseudomonas aeruginosa

Genomic self-targeting of P. aeruginosa PAO1^{IC} was achieved by electroporating cells with pHERD30T (or pHERD20T) expressing the self-targeting spacer of choice. Cells were plated onto LB agar plates containing the selective antibiotic, without inducers, and grown overnight. Single colonies were then grown in liquid LB media containing the selective antibiotic, as well as IPTG to induce the genomic expression of the I-C system genes, and arabinose to induce the expression of the crRNA from the plasmid. The aca1-containing crRNA plasmids do not need additional inducers, as the pBAD promoter controls aca1. Cultures were grown at 37° C. in a shaking incubator overnight to saturation, then plated onto LB agar plates containing the selecting antibiotic, as well as the inducers, and incubated overnight again at 37° C. The resulting colonies were then analyzed individually using colony PCR for any differences at the targeted genomic site compared to a wild-type cell. gDNA was isolated by resuspending 1 colony in 20 μl of H₂O, followed by incubation at 95° C. for 15 min. 1-2 μl of boiled sample was used for PCR. The primers used to assay the targeted sites were designed to amplify genomic regions 1.5-3 kb in size. In the event of a PCR product equal to or smaller than the wild-type fragment (as was often observed when analyzing Cas9-targeted cells), Sanger sequencing (Quintara Biosciences) was used to determine any modifications of the targeted sequences. In some cases, additional analysis of the crRNA-expressing plasmids of the surviving colonies was also performed, by isolating and reintroducing the plasmids into the original I-C CRISPR-Cas strain, where functional self-targeting could be determined based on a significant increase in the lag time of induced cultures, characteristic of self-targeting events.

Escherichia coli

Genomic self-targeting of E. coli was conducted in a similar fashion as P. aeruginosa, except using the pCas3cRh all-in-one vector. Electrocompetent E. coli cells were transformed with pCas3cRh expressing a crRNA targeting the genome. Individual transformants were selected and grown in liquid LB media containing the selecting antibiotic (gentamicin) overnight without any inducers added. The overnight cultures were then plated in the presence of inducer and X-gal to screen for functional lacZ (LB agar+15 μg/ml gentamicin+0.1% rhamnose+1 mM IPTG+20 μg/ml X-gal) and blue/white colonies were counted the next day.

Pseudomonas syringae

Electrocompetent P. syringae cells were also transformed with pCas3cRh plasmids targeting selected genomic sequences. Initial transformants were plated onto KB agar+100 μg/ml rifampicin+50 μg/ml gentamicin plates, and incubated at 28° C. overnight. Single colony transformants were then selected and inoculated in KB liquid media supplemented with rifampicin, gentamicin, and 0.1% rhamnose inducer, and grown to saturation in a shaking incubator at 28° C. Cultures were finally plated onto KB agar plates with rifampicin, gentamicin, and rhamnose and incubated at 28° C. Individual colonies were finally assayed with colony PCR to determine the presence of deletions at the targeted genomic sites.

### Iterative Genome Minimization

Iterative targeting to generate multiple deletions in the P. aeruginosa PAO1^{IC} strain was carried out by alternating the pHERD30T and pHERD20T plasmids each expressing different crRNAs targeting the genome. Each crRNA designed to target the genome was cloned into both the pHERD30T plasmid, which confers gentamicin resistance, as well as the pHERD20T plasmid, which confers carbenicillin resistance. After first transforming and targeting with a pHERD30T plasmid expressing a specific crRNA, deletion candidate isolates were transformed with a pHERD20T expressing a crRNA targeting a different genomic region. As the two plasmids are identical with the exception of the resistance marker, this eliminated the necessity for curing of the original plasmid to be able to target a different region. For the next targeting event, the pHERD30T plasmid could again be used, this time expressing another crRNA targeting a different genomic region. In this manner, pHERD30T and pHERD20T could be alternated to achieve multiple deletions in a rapid process. At each new transformation step, the cells were checked for any residual resistance to the given antibiotic from a previous cycle. Additionally, functionality of the CRISPR-Cas system of the edited cells could be determined through the introduction of a plasmid expressing crRNA targeting the D3 bacteriophage (45), then performing a phage spotting assay to see if phage targeting was occurring or not.

### Measurement of Growth Rates

Pseudomonas aeruginosa

Growth dynamics of various strains were measured using a Synergy 2 automated 96-well plate reader (Biotek Instruments) and the accompanying Gen5 software (Biotek Instruments). Individual colonies were picked and grown overnight in 300 μl volumes of LB in 96-well deep-well plates at 37° C. The grown cultures were then diluted 100-fold into 100 μl of fresh LB in a 96-well clear microtitre plate (Costar) and sealed with Microplate sealing adhesive (Thermo Scientific). Small holes were punched in the sealing adhesive for each well for increased aeration. Doubling times were calculated as described previously (69).

Pseudomonas syringae

To test bacterial growth in planta, we used the Arabidopsis thaliana ecotype Columbia (Col-0), which has previously been shown to be susceptible to infection by P. syringae DC3000. Plants were grown for 5-6 weeks in 9 h light/15 h darkness and 65% humidity. For each inoculum, we measured bacterial growth in 10 individual Col-0 plants. Four leaves from each plant were infiltrated at OD₆₀₀=0.0002, and cored with a #3 borer. The four cores from each plant were then ground, resuspended in 10 mM MgCl₂ and plated in a dilution series on selective media for colony counts at both the time of infection and 3 days post-infection.

To test bacterial growth in vitro, we used both KB and plant apoplast mimicking minimal media (MM) (57). Overnight cultures were prepared from single colonies of each strain, washed, and diluted to OD₆₀₀=0.1 in 96-well plates using either KB or MM. Plates were incubated with shaking at 28° C. OD₆₀₀ was measured over the course of 24-25 hours using an Infinate 200 Pro automated plate reader (Tecan). Statistical analysis determined significantly different groups based on ANOVA analysis on the day 0 group of values and the day 3 group of values. Significant ANOVA results (p<0.01) were further analyzed with a Tukey's HSD post hoc test to generate adjusted p-values for each pairwise comparison. A significance threshold of 0.01 was used to determine which treatment groups were significantly different.

### Bacteriophage Plaque (Spot) Assays

Bacteriophage plaque assays were performed using 1.5% LB agar plates supplemented with 10 mM MgSO4 and the appropriate antibiotic (gentamicin or carbenicillin, depending on the plasmid used to express the crRNA), and 0.7% LB top agar supplemented with 0.5 mM IPTG and 0.1% arabinose inducers added covering the whole plate. 150 μl of the appropriate overnight cultures was suspended in 4 ml molten top agar poured onto an LB agar plate leading to the growth of a bacterial lawn. After 10-15 minutes at room temperature, 3 μl of ten-fold serial dilutions of bacteriophage was spotted onto the solidified top agar. Plates were incubated overnight at 30° C. and imaged the following day using a Gel Doc EZ Gel Documentation System (BioRad) and Image Lab (BioRad) software. The following bacteriophage were used in this study: bacteriophage JBD30 (45), bacteriophage D3 (71), and bacteriophage DMS3m (72).

### Whole-Genome Sequencing

Genomic DNA for whole-genome sequencing (WGS) analysis was isolated directly from bacterial colonies using the Nextera DNA Flex Microbial Colony Extraction kit (Illumina) according to the manufacturer's protocol. Genomic DNA concentration of the samples was determined using a DS-11 Series Spectrophotometer/Fluorometer (DeNovix) and all fell into the range of 200-500 ng/μl. Library preparation for WGS analysis was done using the Nextera DNA Flex Library Prep kit (Illumina) according to the manufacturer's protocol starting from the tagment genomic DNA step. Tagmented DNA was amplified using Nextera DNA CD Indexes (Illumina). Samples were placed overnight at 4° C. following the tagmented DNA amplification step, then continued the next day with the library clean up steps. Quality control of the pooled libraries was performed using a 2100 Bioanalyzer Instrument (Agilent Technologies) with a High Sensitivity DNA Kit (Agilent Technologies). Samples were sequenced using an MiSeq Reagent Kit v2 (Illumina) for a 150 bp paired-end sequencing run using the MiSeq sequencer (Illumina).

Genome sequence assembly was performed using Geneious Prime software version 2019.1.3. Paired read data sets were trimmed using the BBDuk (Decontamination Using Kmers) plugin using a minimum Q value of 20. The genome for the ancestral PAO1^{IC} strain was de novo assembled using the default automated sensitivity settings offered by the software. The consensus sequence of PAO1^{IC} assembled in this manner was then used as the reference sequence for mapping all of the PAO1^{IC} strains with multiple deletions. As a control, the sequences were also mapped to the reference P. aeruginosa PAO1 sequence (NC_002516) to verify deletion border coordinates. Coverage of these sequenced strains ranged from 66 to 143-fold. The sequenced P. aeruginosa environmental strains were also mapped to the PAO1 (NC_002516) reference, while the sequenced E. coli strains were mapped to the E. coli K-12 MG1655 reference sequence (NC_000913). Finally, sequenced P. syringae strains were mapped to the P. syringae DC3000 (NC_004578) reference sequence, along with the pDC3000A endogenous 73.5 kb plasmid sequence (NC_004633). All of these remaining sequenced strains had >100-fold coverage. All deletion junction sequences were manually verified by the presence of multiple reads spanning the deletions, containing sequences from both end boundaries.

WGS data was visualized using the BLAST Ring Image Generator (BRIG) tool (73) employing BLAST+ version 2.9.0. In several cases, short sequences were aligned within previously determined large deletions at redundant sequences such as transposase genes. Such misrepresentations created by BRIG were manually removed to reflect the actual sequencing data.

TABLE 1
Extended, non-essential regions (XNES) of P. aeruginosa PA01
genome with contiguous, individually non-essential genes in
a complex laboratory medium exceeding 100 kb. Data based on
a transposon sequencing dataset from Turner et al. (37).

| Region | Coordinates | Size |
|---|---|---|
| XNES 1 | 27535-142359 | 114 kb |
| XNES 2 | 143267-371151 | 228 kb |
| XNES 3 | 491900-606160 | 114 kb |
| XNES 4 | 841825-986817 | 145 kb |
| XNES 5 | 1147815-1249907 | 102 kb |
| XNES 6 | 1260442-1491913 | 232 kb |
| XNES 7 | 1974210-2150828 | 176 kb |
| XNES 8 | 2216121-2375804 | 160 kb |
| XNES 9 | 2376541-2923367 | 546 kb |
| XNES 10 | 2972700-3079197 | 106 kb |
| XNES 11 | 3155072-3309411 | 154 kb |
| XNES 12 | 3587303-3802567 | 216 kb |
| XNES 13 | 3897357-4062426 | 165 kb |
| XNES 14 | 4294208-4457362 | 163 kb |
| XNES 15 | 4576324-4753990 | 178 kb |
| XNES 16 | 6025305-6180942 | 156 kb |

TABLE 2
Genomic coordinates and extent of homologous sequences at genomic
deletion junctions of whole-genome sequenced self-targeting strains of
P. aeruginosa, P. syringae, and E. coli.

| | Targeted genomic | | | |
|---|---|---|---|---|
| | region (gene, | | | Deletion |
| Sequenced | genomic | Boundary | Boundary | Size |
| strain | coordinate) | 1 | 2 | (bp) |
| phzm_1 | phzM, 4713388 | 4680552 | 4733325 | 52773 |
| phzm_2 | phzM, 4713388 | 4663824 | 4723907 | 60083 |
| phzm_3 | phzM, 4713312 | 4706070 | 4729547 | 23477 |
| PA01delta6_1 | XNES1, 94970 | 85399 | 123464 | 38065 |
| | XNES2, 257270 | 231531 | 276484 | 44953 |
| | XNES6, 1376181 | 1362672 | 1380345 | 17673 |
| | XNES8, 2296157 | 2020556 | 2445438 | 424882 |
| | XNES9, 2650253 | 2562926 | 2681835 | 118909 |
| | phzM, 4713388 | 4654469 | 4742561 | 88092 |
| PA01delta6_2 | XNES1, 94970 | 64555 | 122559 | 58004 |
| | XNES2, 257270 | 222590 | 275151 | 52561 |
| | XNES6, 1376181 | 1360915 | 1394851 | 33936 |
| | XNES8, 2296157 | 2231695 | 2443408 | 211713 |
| | XNES9, 2650253 | 2564130 | 2702613 | 138483 |
| | phzM, 4713388 | 4682907 | 4720607 | 37700 |
| PA01delta6_3 | XNES1, 94970 | 76494 | 118909 | 42415 |
| | XNES2, 257270 | 233283 | 272435 | 39152 |
| | XNES6, 1376181 | 1319223 | 1395353 | 76130 |
| | XNES8, 2296157 | 2259967 | 2440505 | 180538 |
| | XNES9, 2650253 | 2532587 | 2722551 | 189964 |
| | phzM, 4713388 | 4656623 | 4728039 | 71416 |
| PA01delta6_4 | XNES1, 94970 | 86641 | 112742 | 26101 |
| | XNES2, 257270 | 215339 | 272253 | 56914 |
| | XNES6, 1376181 | 1358919 | 1390458 | 31539 |
| | XNES8, 2296157 | 2138166 | 2403535 | 265369 |
| | XNES9, 2650253 | 2617788 | 2671441 | 53653 |
| | phzM, 4713388 | 4706938 | 4722251 | 15313 |
| PA01delta6_5 | XNES1, 94970 | 86283 | 112199 | 25916 |
| | XNES2, 257270 | 215340 | 272255 | 56915 |
| | XNES6, 1376181 | 1368176 | 1455904 | 87728 |
| | XNES8, 2296157 | 2244758 | 2441249 | 196491 |
| | XNES9, 2650253 | 2448500 | 2694293 | 245793 |
| | phzM, 4713388 | 4707045 | 4722269 | 15224 |
| PA01delta6_6 | XNES1, 94970 | 83381 | 183076 | 99695 |
| | XNES2, 257270 | Not | | |
| | | detected | | |
| | | (false | | |
| | | positive) | | |
| | XNES6, 1376181 | 1360919 | 1394270 | 33351 |
| | XNES8, 2296157 | 2201252 | 2368014 | 166762 |
| | XNES9, 2650253 | 2622508 | 2685224 | 62716 |
| | phzM, 4713388 | 4639606 | 4734578 | 94972 |
| PA01delta10 | XNES1, 94970 | 86283 | 112199 | 25916 |
| | XNES2, 257270 | 215340 | 272255 | 56915 |
| | XNES5, 1196780 | 1172779 | 1238033 | 65254 |
| | XNES6, 1376181 | 1368176 | 1455904 | 87728 |
| | XNES8, 2296157 | 2244758 | 2441249 | 196491 |
| | XNES9, 2650253 | 2448500 | 2694293 | 245793 |
| | XNES12, 3695108 | 3682194 | 3720621 | 38427 |
| | XNES13, 3979615 | 3974844 | 3981902 | 7058 |
| | XNES14, 4375854 | 4311169 | 4418478 | 107309 |
| | phzM, 4713388 | 4707045 | 4722269 | 15224 |
| P. syringae | cluster VI, 1513491 | 1447130 | 1547826 | 100696 |
| delta VI | | | | |
| P. syringae | cluster IV, 947715 | 915141 | 981398 | 66257 |
| delta IV IX | | | | |
| | cluster IX, 5352827 | 5310219 | 5365737 | 55518 |
| P. syringae | cluster X, found | plasmid | | |
| delta X | on endogenous | eliminated | | |
| | 73 kb plasmid | from | | |
| | | strain | | |
| E. colli | lacZ, 366203 | 357501 | 375104 | 17603 |
| delta lacZ_2 | | | | |
| E. colli | lacZ, 365514 | 349870 | 377291 | 27421 |
| delta lacZ_3 | | | | |
| E. colli | lacZ, 365480 | 349730 | 375173 | 25443 |
| delta lacZ_4 | | | | |
| E. coli | pdeL, 333128 | 274851 | 381629 | 106778 |
| delta pdeL_1 | | | | |
| E. coli | pdeL, 332824 | 271798 | 381626 | 109828 |
| delta pdeL_2 | | | | |
| P. aeruginosa | phzM, 4713388 | 4699988 | 4733706 | 33718 |
| F11_1 | | | | |
| P. aeruginosa | phzM, 4713388 | 4693410 | 4732374 | 38964 |
| F11_2 | | | | |

TABLE 3
Summary of HR-mediated genome editing experiments using the Type I-F CRISPR-Cas3 system.
Genes were targeted for deletion in the strains PA14 and z8. Experiments targeted 4 single
genes and 2 gene blocks, teg and rebB, that comprise X and Y genes, respectively.

| | Edited | HR edits | Nontemplated | No edits | | Designed | HR template length |
|---|---|---|---|---|---|---|---|
| Strain | gene | (%) | edits (%) | (%) | n | deletion (bp) | (left + right, bp) |
| PA14 | psiF | 100 | 0 | 0 | 12 | 0.5 | 600 + 600 |
| PA14 | rebB | 50 | 50 | 0 | 16 | 4.1 | 600 + 600 |
| z8 | ghlO | 100 | 0 | 0 | 5 | 0.2 | 600 + 600 |
| z8 | mexZ | 75 | 25 | 0 | 12 | 0.6 | 722 + 600 |
| z8 | psiF | 100 | 0 | 0 | 12 | 0.5 | 600 + 600 |
| z8 | qsrO | 29(80)* | 71 | 0 | 14 | 0.4 | 751 + 596 |
| z8 | teg | 75 | 25 | 0 | 4 | 6.3 | 800 + 809 |

Transformants were classified as
1) ‘HR edits’ that have the HR designed deletion;
2) ‘non-templated edits’ that have a non-designed deletion encompassing the targeted gene,
3) ‘no edits’ where the targeted gene is intact.
*two colony morphologies with different editing frequencies were obtained in this experiment.

TABLE 4A
DNA oligonucleotides used in this study for P. aeruginosa

| Oligo name | Sequence (5′-3′) | Description |
|---|---|---|
| phzm_FWD_spacer_1 | GAAAC CTGCAATGCCGGAGGTTGTAGCCAAGTTGTAATT G | spacer targeting leading strand at |
| | | phzM |
| phzm_REV_spacer_1 | GGACGTTACGGCCTCCAACATCGGTTCAACATTAA CAGCG | spacer targeting leading strand at |
| | | phzM |
| phzm_FWD_spacer_2 | GAAAC GGTACGCAGGAAAAGGCTCTGGAACAGGCAGTTG G | spacer targeting lagging strand at |
| | | phzM |
| phzm_REV_spacer_2 | G CCATGCGTCCTTTTCCGAGACCTTGTCCGTCAAC CAGCG | spacer targeting lagging strand at |
| | | phzM |
| phzM_chk_F | gcggaacggctattcccaatg | primer for checking deletion at |
| | | phzM |
| phzM_chk_R | acttcgagatccagggctacc | primer for checking deletion at |
| | | phzM |
| Region_1_FWD_ | gaaacGGCGAGTACGTAGACATGCCGGAAGACCATCTCGg | spacer targeting leading strand at |
| spacer_1 | | XNES1 |
| Region_1_REV_ | gcgacCGAGATGGTCTTCCGGCATGTCTACGTACTCGCCg | spacer targeting leading strand at |
| spacer_1 | | XNES1 |
| Region_1_FWD_ | gaaacctcggcccgctgctgcgcctgggcaactacgaacg | spacer targeting lagging strand at |
| spacer_2 | | XNES1 |
| Region_1_REV_ | gcgacgttcgtagttgcccaggcgcagcagcgggccgagg | spacer targeting lagging strand at |
| spacer_2 | | XNES1 |
| R1_chk-FWD | CTGCTGGTACAGCTCCTGGATG | primer for checking deletion at |
| | | XNES1 |
| R1_chk-REV | GCGAGTACGAGCACGAACTGTC | primer for checking deletion at |
| | | XNES1 |
| Region_2_FWD_ | gaaacCTCGGCTGCGCCAACCAGGCCGGCGAGGACAACCg | spacer targeting leading strand at |
| spacer_1 | | XNES2 |
| Region_2_REV_ | gcgacGGTTGTCCTCGCCGGCCTGGTTGGCGCAGCCGAGg | spacer targeting leading strand at |
| spacer_1 | | XNES2 |
| Region_2_FWD_ | gaaacAGCGGCACCGCCGCGAGGTCGTCGGCGCGCACCGg | spacer targeting lagging strand at |
| spacer_2 | | XNES2 |
| Region_2_REV_ | gcgacCGGTGCGCGCCGACGACCTCGCGGCGGTGCCGCTg | spacer targeting lagging strand at |
| spacer_2 | | XNES2 |
| R2_chk-FWD | TGACTCCCGACCTGGTCTAC | primer for checking deletion at |
| | | XNES2 |
| R2_chk-REV | CGACAGGGTCCGTTTCATCC | primer for checking deletion at |
| | | XNES2 |
| R5_FWD_spacer_1 | gaaaccgcgacaagggcaagaacgtattgctgctgatggg | spacer targeting leading strand at |
| | | XNES5 |
| R5_REV_spacer_1 | gcgacccatcagcagcaatacgttcttgcccttgtcgcgg | spacer targeting leading strand at |
| | | XNES5 |
| R5_FWD_spacer_2 | gaaacggcagcttggcgaacaccgatggcggatacccctg | spacer targeting lagging strand at |
| | | XNES5 |
| R5_REV_spacer_2 | gcgacaggggtatccgccatcggtgttcgccaagctgccg | spacer targeting lagging strand at |
| | | XNES5 |
| R5_chk_F | TTCGAGCAACAGCGCGAAC | primer for checking deletion at |
| | | XNES5 |
| R5_chk_R | TCGGGACACAACAGCTAC | primer for checking deletion at |
| | | XNES5 |
| Region_6_FWD_ | gaaacCTGGCACGCGCCCATGCCGCAGAACGGCGCGCGCg | spacer targeting leading strand at |
| spacer_1 | | XNES6 |
| Region_6_REV_ | gcgacGCGCGCGCCGTTCTGCGGCATGGGCGCGTGCCAGg | spacer targeting leading strand at |
| spacer_1 | | XNES6 |
| Region_6_FWD_ | gaaacATCGACCGGCGTCCGCTGCGGGTCGCCGTCGGTAg | spacer targeting lagging strand at |
| spacer_2 | | XNES6 |
| Region_6_REV_ | gcgacTACCGACGGCGACCCGCAGCGGACGCCGGTCGATg | spacer targeting lagging strand at |
| spacer_2 | | XNES6 |
| R6_GC2_chk_F | GAGGTAGCCACTGTTGTTGAAG | primer for checking deletion at |
| | | XNES6 |
| R6_GC2_chk_R | GAAACCGTAGGACGCATGATTG | primer for checking deletion at |
| | | XNES6 |
| Region_8_FWD_ | gaaacCGCGACCCCGCCGTGCGCCATGCGATGTGCGAGGg | spacer targeting leading strand at |
| spacer_1 | | XNES8 |
| Region_8_REV_ | gcgacCCTCGCACATCGCATGGCGCACGGCGGGGTCGCGg | spacer targeting leading strand at |
| spacer_1 | | XNES8 |
| Region_8_FWD_ | gaaacCAGCGCCTGCGGGTGGTAGATGTCGCGGCCCTGGg | spacer targeting lagging strand at |
| spacer_2 | | XNES8 |
| Region_8_REV_ | gcgacCCAGGGCCGCGACATCTACCACCCGCAGGCGCTGg | spacer targeting lagging strand at |
| spacer_2 | | XNES8 |
| R8_chk2_F | AGCCTCTGAGCGGCACTTTC | primer for checking deletion at |
| | | XNES8 |
| R8_chk2_R | AGTCGTCGAGCCGGTAATCC | primer for checking deletion at |
| | | XNES8 |
| Region_9_FWD_ | gaaacGACCGCAACCCGGGCGAAGCGGTGGACTGGCATCg | spacer targeting leading strand at |
| spacer_1 | | XNES9 |
| Region_9_REV_ | gcgacGATGCCAGTCCACCGCTTCGCCCGGGTTGCGGTCg | spacer targeting leading strand at |
| spacer_1 | | XNES9 |
| Region_9_FWD_ | gaaacGGCGCGGAGCGACTGGGCAGCGGAAAGCAGCGGCg | spacer targeting lagging strand at |
| spacer_2 | | XNES9 |
| Region_9_REV_ | gcgacGCCGCTGCTTTCCGCTGCCCAGTCGCTCCGCGCCg | spacer targeting lagging strand at |
| spacer_2 | | XNES9 |
| R9_chk2_F | gcaagttcgccatcgtcatgag | primer for checking deletion at |
| | | XNES9 |
| R9_chk2_R | gaaccgccatgcacgcattatc | primer for checking deletion at |
| | | XNES9 |
| Region_12_FWD_ | gaaacGGAATTGTCGCAGATTTGAGCGGAAGAGGACGAAg | spacer targeting leading strand at |
| spacer_1 | | XNES12 |
| Region_12_REV_ | gcgacTTCGTCCTCTTCCGCTCAAATCTGCGACAATTCCg | spacer targeting leading strand at |
| spacer_1 | | XNES12 |
| Region_12_FWD_ | gaaacTCCCGTCCTCCGCGACTGCGGCACGCTCACAGCAg | spacer targeting lagging strand at |
| spacer_2 | | XNES12 |
| Region_12_REV_ | gcgacTGCTGTGAGCGTGCCGCAGTCGCGGAGGACGGGAg | spacer targeting lagging strand at |
| spacer_2 | | XNES12 |
| R12_chk_F | CAGCATCTGCAGGATCAC | primer for checking deletion at |
| | | XNES12 |
| R12_chk_R | GTGATCGTCACCGAAGTC | primer for checking deletion at |
| | | XNES12 |
| Region_13_FWD_ | gaaacACGGGGAGCGGACATCGAGTATTAATGAACCCTTg | spacer targeting leading strand at |
| spacer_1 | | XNES13 |
| Region_13_REV_ | gcgacAAGGGTTCATTAATACTCGATGTCCGCTCCCCGTg | spacer targeting leading strand at |
| spacer_1 | | XNES13 |
| Region_13_FWD_ | gaaacATGGAAACATGGGGAGGGCCAGGGAAAGTCAATCg | spacer targeting lagging strand at |
| spacer_2 | | XNES13 |
| Region_13_REV_ | gcgacGATTGACTTTCCCTGGCCCTCCCCATGTTTCCATg | spacer targeting lagging strand at |
| spacer_2 | | XNES13 |
| R13_chk_F | GTTCCAGCAGACCATCAAG | primer for checking deletion at |
| | | XNES13 |
| R13_chk_R | TGAAACCGGGCTCGATAAC | primer for checking deletion at |
| | | XNES13 |
| Region_14_FWD_ | gaaacCTGCAGCGGATCGTCTACGAGTACTGCGCCGCGGg | spacer targeting leading strand at |
| spacer_1 | | XNES14 |
| Region_14_REV_ | gcgacCCGCGGCGCAGTACTCGTAGACGATCCGCTGCAGg | spacer targeting leading strand at |
| spacer_1 | | XNES14 |
| Region_14_FWD_ | gaaacACCTTGGCCCGTGCCCAGGGCCTGGGCACGCCGAg | spacer targeting lagging strand at |
| spacer_2 | | XNES14 |
| Region_14_REV_ | gcgacTCGGCGTGCCCAGGCCCTGGGCACGGGCCAAGGTg | spacer targeting lagging strand at |
| spacer_2 | | XNES14 |
| R14_chk_F | AGCGAGCTGGACGAAATC | primer for checking deletion at |
| | | XNES14 |
| R14_chk_R | TAACCGCTTGCGGCTATC | primer for checking deletion at |
| | | XNES14 |
| rplQ_FWD_spacer_ | gaaacTGGAACATAGCCTTGCGGTGCGCGCTGGTGCGGCg | spacer targeting leading strand at |
| 1 | | rplQ |
| rplQ_REV_spacer_ | gcgacGCCGCACCAGCGCGCACCGCAAGGCTATGTTCCAg | spacer targeting leading strand at |
| 1 | | rplQ |
| rplQ_FWD_spacer_ | gaaacGAACACGAACTGATCAAAACCACCCTGCCCAAGGg | spacer targeting lagging strand at |
| 2 | | rplQ |
| rplQ_REV_spacer_ | gcgacCCTTGGGCAGGGTGGTTTTGATCAGTTCGTGTTCg | spacer targeting lagging strand at |
| 2 | | rplQ |
| rplQ-chk-FWD | TTCGGCAGCTTCTACGAC | primer for checking deletion at |
| | | rplQ |
| rplQ-chk-REV | TCGAGATCCTGCTGAACC | primer for checking deletion at |
| | | rplQ |
| D3_Fwd | gaaacACGATTGCGGACATGGCAGGCTGCCGCTGCTGGAg | spacer targeting D3 phage |
| D3_Rev | gcgacTCCAGCAGCGGCAGCCTGCCATGTCCGCAATCGTg | spacer targeting D3 phage |
| ArraycrRNA1_FWD_ | aattcGTCGCGCCCCGCACGGGCGCGTGGATTGAAACgagaccT | Wild-type IC crRNA entry sequence |
| LL | CTCTGGACAAAggtctcGTCGCGCCCCGCACGGGCGCGTGGAT | with BsaI site |
| | TGAAACa | |
| ArraycrRNA1_REV_ | AgcttGTTTCAATCCACGCGCCCGTGCGGGGCGCGACgagaccT | Wild-type IC crRNA entry sequence |
| LL | TTGTCCAGAGAggtctcGTTTCAATCCACGCGCCCGTGCGGGGC | with BsaI site |
| | GCGACg | |
| ArraycrRNA1_FWD_ | aattcGTCGCGCCCCGCACGGGCGCGTGGATTGAAACgagaccT | Modified IC crRNA entry sequence |
| BC | CTCTGGACAAAggtctc | with BsaI site |
| | GTCGCCCGGCAAAACCGGGCGTGGATTGAAACa | |
| ArraycrRNA1_REV_ | AgcttGTTTCAATCCACGCCCGGTTTTGCCGGGCGAC | Modified IC crRNA entry sequence |
| BC | gagaccTTTGTCCAGAGAggtctcGTTTCAATCCACGCGCCCGTG | with BsaI site |
| | CGGGGCGCGACg | |
| phzM_LD_Up_F | ctgctctgcgaggctggccgataag | primer for generating upstream |
| | ACAGCAGCACCGGTTTCCAG | homology for 56.5 kb deletion |
| phzM_LD_Up_R | gggcggctgttccttgtcctgtggg | primer for generating upstream |
| | GGCTACGTGAGTTCGGAGAAG | homology for 56.5 kb deletion |
| phzM_LD_Down_F | ggcccttctccgaactcacgtagcc | primer for generating downstream |
| | CCCACAGGACAAGGAACAG | homology for 56.5 kb deletion |
| phzM_LD_Down_R | cttttgctggccttttgctcacataag | primer for generating downstream |
| | ATTGGCGTCCCGCATCGATCTC | homology for 56.5 kb deletion |
| phzM_SD_Up_F | ctgctctgcgaggctggccgataag CGTAGAACAGCACCATGTC | primer for generating upstream |
| | | homology for 0.17 kb deletion |
| phzM_SD_Up_R | tgtttcaaatagccagcatccctgg GGAACAGGCAGTTGGAAAG | primer for generating upstream |
| | | homology for 0.17 kb deletion |
| phzM_SD_Down_F | ctggaactttccaactgcctgttcc CCAGGGATGCTGGCTATTTG | primer for generating downstream |
| | | homology for 0.17 kb deletion |
| phzM_SD_Down_R | tttgctggccttttgctcacataag GCTTTCCGTGGTCCAGTTG | primer for generating downstream |
| | | homology for 0.17 kb deletion |

TABLE 4B
DNA oligonucleotides used in this study for P. syringae

| Oligo name | Sequence (5′-3′) | Description |
|---|---|---|
| p30Rha-f | agtgctctgcaggaattcctcgagaAGGGAGCGCACCTATGGAC | Amplification of cas3, cas5, |
| | | and cas8(1) and annealing to |
| | | p30T |
| cas8-cas7 | cggcctgttcggacACCGGAGCATTTTCCCCC | Amplification of cas3, cas5, |
| | | and cas8(1) and annealing to |
| | | cas8(2)and cas7 |
| cas3-cas5-cas8 | aaaatgctcggtgTCCGAACAGGCCGCCTTT | Amplification of cas8(2) and |
| | | cas7 and annealing to cas3, |
| | | cas5, and cas8(1) |
| p30Rha-r | ggaatccccgtcgacggtatcgataCCTGAAACTAGAGGTACTC | Amplification of cas8(2) and |
| | GCGC | cas7 and annealing to p30T |
| p30Rha_Cas_ICmr_ | ctagGTCGCGCCCCGCACGGGCGCGTGGATTGAAACgagaccTCTC | Modified IC crRNA entry |
| Fwd | TGGACAAAggtctcGTCGCCCGGCAAAACCGGGCGTGGATTGAAAC | sequence into p30T-Rha-IC |
| | | plasmid with BsaI site |
| p30Rha_Cas_ICmr_ | ctagGTTTCAATCCACGCCCGGTTTTGCCGGGCGAC | Modified IC crRNA entry |
| Rev | gagaccTTTGTCCAGAGAggtctcGTTTCAATCCACGCGCCCGTGC | sequence into p30T-Rha-IC |
| | GGGGCGCGAC | plasmid with BsaI site |
| p30Rha_seq_Fwd | TGCGGTGAGCATCACATC | Sequencing primer for crRNA |
| | | cloning into p30T-Rha_IC |
| | | plasmid |
| p30Rha_seq_Rev | ATACGCCGCTGAAACTCG | Sequencing primer for crRNA |
| | | cloning into p30T-Rha_IC |
| | | plasmid |
| VI_target_F | GAAACATCCACGACCCGAACCGTATCCACGGCCATCTGGG | spacer targeting cluster VI |
| VI_target_R | gcgacCCAGATGGCCGTGGATACGGTTCGGGTCGTGGATg | spacer targeting cluster VI |
| IVIX_target_F | GAAACCTTGACCTCGGGTGGAATACCGGAGGCGGCGCCAG | spacer targeting clusters IV |
| | | and IX |
| IVIX_target_R | gcgacTGGCGCCGCCTCCGGTATTCCACCCGAGGTCAAGg | spacer targeting cluster IV |
| | | and IX |
| X_target_F | GAAACGTTTGGGCAGACGGATGATTAACCGGATTGTGACG | spacer targeting cluster X |
| X_target_R | gcgacGTCACAATCCGGTTAATCATCCGTCTGCCCAAACg | spacer targeting cluster X |
| c1_chk_FWD | TCTGCCAGTTCGCAAACG | deletion checking primer at |
| | | cluster VI |
| c1_chk_REV | AAGCGCCGCATTGAAGTG | deletion checking primer at |
| | | cluster VI |
| c2_chk_FWD | CGCATCAATCGGCCAGAATAG | deletion checking primer at |
| | | cluster IV |
| c2_chk_REV | GAATACGTTCGGCCAATGGAG | deletion checking primer at |
| | | cluster IV |
| c2_chk(alt)_F | CCGTGCATATCGGATCAGTC | deletion checking primer at |
| | | cluster IX |
| c2_chk(alt)_R | GCACAGCCAGGTCTTGATAC | deletion checking primer at |
| | | cluster IX |
| c3_chk_Fwd | GTCAGCAATCACTCGATACC | deletion checking primer at |
| | | cluster X |
| c3_chk_Rev | TCGCTTTGAAGGCATGAC | deletion checking primer at |
| | | cluster X |

TABLE 4C
DNA oligonucleotides used in this study for E. coli

| Sequence (5′-3′) | Description |
|---|---|
| gaaacgccagctggcgtaatagcgaagaggcccgcaccgg | spacer targeting leading strand at lacZ |
| gcgaccggtgcgggcctcttcgctattacgccagctggcg | spacer targeting leading strand at lacZ |
| gaaacaccctgccataaagaaactgttacccgtaggtagg | spacer targeting lagging strand at lacZ |
| gcgacctacctacgggtaacagtttctttatggcagggtg | spacer targeting lagging strand at lacZ |
| gaaacggcggtgaaattatcgatgagcgtggtggttatgg | spacer targeting lagging strand at lacZ |
| gcgaccataaccaccacgctcatcgataatttcaccgccg | spacer targeting lagging strand at lacZ |
| TGATGTGCCCGGCTTCTGAC | primer for checking deletion at lacZ |
| GACCGCTTGCTGCAACTCTC | primer for checking deletion at lacZ |
| gaaacATCTTAATTTTGCTGACACCCGCGCTCATTTACAg | spacer targeting leading strand at yaiS |
| gcgacTGTAAATGAGCGCGGGTGTCAGCAAAATTAAGATg | spacer targeting leading strand at yaiS |
| gaaacCATCTGTGCCAGAGTTGCCGGTAGTCATCACCACg | spacer targeting lagging strand at yaiS |
| gcgacGTGGTGATGACTACCGGCAACTCTGGCACAGATGg | spacer targeting lagging strand at yaiS |
| gaaacATCAGCACAACATTACCTTTGCGCTGGATGACTTg | spacer targeting leading strand at pdeL |
| gcgacAAGTCATCCAGCGCAAAGGTAATGTTGTGCTGATg | spacer targeting leading strand at pdeL |
| gaaacCCGTTTGTGGATGTTCCCAGCGGACAAGCACCTCg | spacer targeting lagging strand at pdeL |
| gcgacGAGGTGCTTGTCCGCTGGGAACATCCACAAACGGg | spacer targeting lagging strand at pdeL |
| gaaacGCCGCTACGTCACTGGCAGGCCGGGCCGGGTAAAg | spacer targeting leading strand at yahK |
| gcgacTTTACCCGGCCCGGCCTGCCAGTGACGTAGCGGCg | spacer targeting leading strand at yahK |
| gaaacGCGTTTTGCCTCAGAAGTGGTAAATGCCACCACAG | spacer targeting lagging strand at yahK |
| gcgacTGTGGTGGCATTTACCACTTCTGAGGCAAAACGCg | spacer targeting lagging strand at yahK |
| gaaacGCCTGACGCGCGCCCTGAACCACTGCCAGACCAAg | spacer targeting leading strand at frmA |
| gcgacTTGGTCTGGCAGTGGTTCAGGGCGCGCGTCAGGCg | spacer targeting leading strand at frmA |
| gaaacAGTGAATACACCGTAGTCGCGGAAGTGTCTCTGGg | spacer targeting lagging strand at frmA |
| gegacCCAGAGACACTTCCGCGACTACGGTGTATTCACTg | spacer targeting lagging strand at frmA |

TABLE 5
Type I-C repeat sequences and citations

| | | LENGTH | |
|---|---|---|---|
| ORGANISM | REPEAT SEQUENCE | (NT) | REFERENCE |
| Pseudomonas | GTCGCGCCCCGCACGGGCGCGTGGATTGAAAC | 32 | Our study |
| aeruginosa | | | |
| Legionella | GTCGCGCCCCGTGCGGGCGCGTGGATTGAAAC | 32 | Rao, C. et al. Active and |
| pneumophila | | | adaptive Legionella |
| | | | CRISPR-Cas reveals a |
| | | | recurrent challenge to the |
| | | | pathogen. Cellular |
| | | | Microbiology 18, 1319-1338 |
| | | | (2016). |
| Desulfovibrio | GTCGCCCCCCACGCGGGGGCGTGGATTGAAAC | 32 | Hochstrasser, M. L., Taylor, |
| vulgaris | | | D. W., Kornfeld, J. E., |
| | | | Nogales, E. & Doudna, J. A. |
| | | | DNA Targeting by a Minimal |
| | | | CRISPR RNA-Guided |
| | | | Cascade. Molecular Cell 63, |
| | | | 840-851 (2016). |
| Eggerthella | GTCACTCCCCGCATGGGGAGTGCGGGTTGAAAT | 33 | Soto-Perez, Paola and |
| lenta | | | Bisanz, Jordan E. and |
| | | | Berry, Joel D. and Lam, |
| | | | Kathy N. and Bondy- |
| | | | Denomy, Joseph and |
| | | | Turnbaugh, Peter, CRISPR- |
| | | | Cas Immune System of a |
| | | | Prevalent Human Gut |
| | | | Bacterium Reveals |
| | | | Hypertargeting Against Gut |
| | | | Virome Phages (April 1, |
| | | | 2019). Available at SSRN: |
| | | | ssrn.com/abstract=3363840 |

### REFERENCES

1. Makarova, K. S. et al. Evolutionary classification of CRISPR-Cas systems: a burst of class 2 and derived variants. Nat. Rev. Microbiol. 18, 67-83 (2020).
2. Barrangou, R. et al. CRISPR provides acquired resistance against viruses in prokaryotes. Science 315, 1709-1712 (2007).
3. Garneau, J. E. et al. The CRISPR/Cas bacterial immune system cleaves bacteriophage and plasmid DNA. Nature 468, 67-71 (2010).
4. Barrangou, R. & Doudna, J. A. Applications of CRISPR technologies in research and beyond. Nat. Biotechnol. 933-941 (2016) doi: 10.1038/nbt.3659.
5. Wiedenheft, B. et al. Structures of the RNA-guided surveillance complex from a bacterial immune system. Nature 477, 486-489 (2011).
6. Westra, E. R. et al. CRISPR immunity relies on the consecutive binding and degradation of negatively supercoiled invader DNA by Cascade and Cas3. Mol. Cell 46, 595-605 (2012).
7. Brouns, S. J. J. et al. Small CRISPR RNAs Guide Antiviral Defense in Prokaryotes. Science 321, 960-964 (2008).
8. Hidalgo-Cantabrana, C. & Barrangou, R. Characterization and applications of Type I CRISPR-Cas systems. Biochem. Soc. Trans. doi: 10.1042/BST20190119.
9. Sinkunas, T. et al. Cas3 is a single-stranded DNA nuclease and ATP-dependent helicase in the CRISPR/Cas immune system. EMBO J. 30, 1335-1342 (2011).
10. Sinkunas, T. et al. In vitro reconstitution of Cascade-mediated CRISPR immunity in Streptococcus thermophilus. EMBO J. 32, 385-394 (2013).
11. Mulepati, S. & Bailey, S. In vitro reconstitution of an Escherichia coli RNA-guided immune system reveals unidirectional, ATP-dependent degradation of DNA target. J. Biol. Chem. 288, 22184-22192 (2013).
12. Hochstrasser, M. L. et al. CasA mediates Cas3-catalyzed target degradation during CRISPR RNA-guided interference. Proc. Natl. Acad. Sci. U.S.A 111, 6618-6623 (2014).
13. Redding, S. et al. Surveillance and Processing of Foreign DNA by the Escherichia coli CRISPR-Cas System. Cell 163, 854-865 (2015).
14. Xiao, Y., Luo, M., Dolan, A. E., Liao, M. & Ke, A. Structure basis for RNA-guided DNA degradation by Cascade and Cas3. Science 361, eaat0839 (2018).
15. Esvelt, K. M. & Wang, H. H. Genome-scale engineering for systems and synthetic biology. Mol. Syst. Biol. 9, (2013).
16. Montalbano, A., Canver, M. C. & Sanjana, N. E. High-Throughput Approaches to Pinpoint Function within the Noncoding Genome. Mol. Cell 68, 44-59 (2017).
17. Makarova, K. S. et al. An updated evolutionary classification of CRISPR-Cas systems. Nat. Rev. Microbiol. 13, 722-736 (2015).
18. Vercoe, R. B. et al. Cytotoxic Chromosomal Targeting by CRISPR/Cas Systems Can Reshape Bacterial Genomes and Expel or Remodel Pathogenicity Islands. PLOS Genet 9, e1003454 (2013).
19. Gomaa, A. A. et al. Programmable removal of bacterial strains by use of genome-targeting CRISPR-Cas systems. mBio 5, e00928-00913 (2014).
20. Kiro, R., Shitrit, D. & Qimron, U. Efficient engineering of a bacteriophage genome using the type I-E CRISPR-Cas system. RNA Biol. 11, 42-44 (2014).
21. Li, Y. et al. Harnessing Type I and Type III CRISPR-Cas systems for genome editing. Nucleic Acids Res. 44, e34-e34 (2016).
22 Pyne, M. E., Bruder, M. R., Moo-Young, M., Chung, D. A. & Chou, C. P. Harnessing heterologous and endogenous CRISPR-Cas machineries for efficient markerless genome editing in Clostridium. Sci. Rep. 6, 25666 (2016).
23. Zhang, J., Zong, W., Hong, W., Zhang, Z.-T. & Wang, Y. Exploiting endogenous CRISPR-Cas system for multiplex genome editing in Clostridium tyrobutyricum and engineer the strain for high-level butanol production. Metab. Eng. doi: 10.1016/j.ymben.2018.03.007.
24. Maikova, A., Kreis, V., Boutserin, A., Severinov, K. & Soutourina, O. Using endogenous CRISPR-Cas system for genome editing in the human pathogen Clostridium difficile. Appl. Environ. Microbiol. AEM.01416-19 (2019) doi: 10.1128/AEM.01416-19.
25. Hidalgo-Cantabrana, C., Goh, Y. J., Pan, M., Sanozky-Dawes, R. & Barrangou, R. Genome editing using the endogenous type I CRISPR-Cas system in Lactobacillus crispatus. Proc. Natl. Acad. Sci. U.S.A 116, 15774-15783 (2019).
26. Hampton, H. G. et al. CRISPR-Cas gene-editing reveals RsmA and RsmC act through FlhDC to repress the SdhE flavinylation factor and control motility and prodigiosin production in Serratia. Microbiology 162, 1047-1058 (2016).
27. Cheng, F. et al. Harnessing the native type I-B CRISPR-Cas for genome editing in a polyploid archaeon. J. Genet. Genomics Yi Chuan Xue Bao 44, 541-548 (2017).
28. Cañez, C., Selle, K., Goh, Y. J. & Barrangou, R. Outcomes and characterization of chromosomal self-targeting by native CRISPR-Cas systems in Streptococcus thermophilus. FEMS Microbiol. Lett. 366, (2019).
29. Zheng, Y. et al. Characterization and repurposing of the endogenous Type I-F CRISPR-Cas system of Zymomonas mobilis for genome engineering. bioRxiv 576355 (2019) doi: 10.1101/576355.
30. Dolan, A. E. et al. Introducing a Spectrum of Long-Range Genomic Deletions in Human Embryonic Stem Cells Using Type I CRISPR-Cas. Mol. Cell 74, 936-950.e5 (2019).
31. Morisaka, H. et al. CRISPR-Cas3 induces broad and unidirectional genome editing in human cells. Nat. Commun. 10, 1-13 (2019).
32. Cameron, P. et al. Harnessing type I CRISPR-Cas systems for genome engineering in human cells. Nat. Biotechnol. 37, 1471-1477 (2019).
33. Pickar-Oliver, A. et al. Targeted transcriptional modulation with type I CRISPR-Cas systems in human cells. Nat. Biotechnol. 1-9 (2019) doi: 10.1038/s41587-019-0235-7.
34 Nam, K. H. et al. Cas5d Protein Processes Pre-crRNA and Assembles into a Cascade-like Interference Complex in Subtype I-C/Dvulg CRISPR-Cas System. Structure 20, 1574-1584 (2012).
35. Hochstrasser, M. L., Taylor, D. W., Kornfeld, J. E., Nogales, E. & Doudna, J. A. DNA Targeting by a Minimal CRISPR RNA-Guided Cascade. Mol. Cell 63, 840-851 (2016).
36. Marino, N. D. et al. Discovery of widespread type I and type V CRISPR-Cas inhibitors. Science 362, 240-242 (2018).
37. Turner, K. H., Wessel, A. K., Palmer, G. C., Murray, J. L. & Whiteley, M. Essential genome of Pseudomonas aeruginosa in cystic fibrosis sputum. Proc. Natl. Acad. Sci. 112, 4110-4115 (2015).
38. Meek, D. W. & Hayward, R. S. Nucleotide sequence of the rpoA-rpIQ DNA of Escherichia coli: a second regulatory binding site for protein S4? Nucleic Acids Res. 12, 5813-5821 (1984).
39 Dillard, K. E. et al. Assembly and Translocation of a CRISPR-Cas Primed Acquisition Complex. Cell (2018) doi: 10.1016/j.cell.2018.09.039.
40. Chayot, R., Montagne, B., Mazel, D. & Ricchetti, M. An end-joining repair mechanism in Escherichia coli. Proc. Natl. Acad. Sci. 107, 2141-2146 (2010).
41. Buell, C. R. et al. The complete genome sequence of the Arabidopsis and tomato pathogen Pseudomonas syringae pv. tomato DC3000. Proc. Natl. Acad. Sci. U.S.A 100, 10181-10186 (2003).
42. Lindeberg, M., Cunnac, S. & Collmer, A. Pseudomonas syringae type III effector repertoires: last words in endless arguments. Trends Microbiol. 20, 199-208 (2012).
43. Kvitko, B. H. et al. Deletions in the repertoire of Pseudomonas syringae pv. tomato DC3000 type III secretion effector genes reveal functional overlap among effectors. PLOS Pathog. 5, e1000388 (2009).
44. Cady, K. C., Bondy-Denomy, J., Heussler, G. E., Davidson, A. R. & O'Toole, G. A. The CRISPR/Cas adaptive immune system of Pseudomonas aeruginosa mediates resistance to naturally occurring and engineered phages. J. Bacteriol. 194, 5728-5738 (2012).
45. Bondy-Denomy, J., Pawluk, A., Maxwell, K. L. & Davidson, A. R. Bacteriophage genes that inactivate the CRISPR/Cas bacterial immune system. Nature 493, 429-432 (2013).
46. Rauch, B. J. et al. Inhibition of CRISPR-Cas9 with Bacteriophage Proteins. Cell 168, 150-158.e10 (2017).
47. Stanley, S. Y. et al. Anti-CRISPR-Associated Proteins Are Crucial Repressors of Anti-CRISPR Transcription. Cell 178, 1452-1464.e13 (2019).
48. Rollins, M. F. et al. Cas1 and the Csy complex are opposing regulators of Cas2/3 nuclease activity. Proc. Natl. Acad. Sci. U.S.A 114, E5113-E5121 (2017).
49. Caliando, B. J. & Voigt, C. A. Targeted DNA degradation using a CRISPR device stably carried in the host genome. Nat. Commun. 6, 6989 (2015).
50. Pósfai, G. et al. Emergent properties of reduced-genome Escherichia coli. Science 312, 1044-1046 (2006).
51. Fehér, T., Papp, B., Pál, C. & Pósfai, G. Systematic Genome Reductions: Theoretical and Experimental Approaches. Chem. Rev. 107, 3498-3513 (2007).
52. Csörgő, B., Nyerges, Á., Pósfai, G. & Fehér, T. System-level genome editing in microbes. Curr. Opin. Microbiol. 33, 113-122 (2016).
53. Képès, F. et al. The layout of a bacterial genome. FEBS Lett. 586, 2043-2048 (2012).
54. Ghosh, S. & O'Connor, T. J. Beyond Paralogs: The Multiple Layers of Redundancy in Bacterial Pathogenesis. Front. Cell. Infect. Microbiol. 7, (2017).
55. Cui, L. & Bikard, D. Consequences of Cas9 cleavage in the chromosome of Escherichia coli. Nucleic Acids Res. gkw223 (2016) doi: 10.1093/nar/gkw223.
56. Kowalczykowski, S. C. & Eggleston, A. K. Homologous Pairing and Dna Strand-Exchange Proteins. Annu. Rev. Biochem. 63, 991-1043 (1994).
57. Bowater, R. & Doherty, A. J. Making Ends Meet: Repairing Breaks in Bacterial DNA by Non-Homologous End-Joining. PLOS Genet 2, e8 (2006).
58. Hnisz, D. et al. Super-enhancers in the control of cell identity and disease. Cell 155, 934-947 (2013).
59. Tuladhar, R. et al. CRISPR-Cas9-based mutagenesis frequently provokes on-target mRNA misregulation. Nat. Commun. 10, 1-10 (2019).
60. Smits, A. H. et al. Biological plasticity rescues target activity in CRISPR knock outs. Nat. Methods 1-7 (2019) doi: 10.1038/s41592-019-0614-5.
61. Choi, K.-H. et al. A Tn7-based broad-range bacterial cloning and expression system. Nat. Methods 2, 443-448 (2005).
62. Stover, C. K. et al. Complete genome sequence of Pseudomonas aeruginosa PAO1, an opportunistic pathogen. Nature 406, 959 (2000).
63. Choi, K.-H. & Schweizer, H. P. mini-Tn7 insertion in bacteria with single attTn7 sites: example Pseudomonas aeruginosa. Nat. Protoc. 1, 153-161 (2006).
64. Blattner, F. R. et al. The complete genome sequence of Escherichia coli K-12. Science 277, 1453-1462 (1997).
65. Qiu, D., Damron, F. H., Mima, T., Schweizer, H. P. & Yu, H. D. PBAD-Based Shuttle Vectors for Functional Analysis of Toxic and Highly Regulated Genes in Pseudomonas and Burkholderia spp, and Other Bacteria. Appl. Environ. Microbiol. 74, 7422-7426 (2008).
66. Gibson, D. G. et al. Enzymatic assembly of DNA molecules up to several hundred kilobases. Nat. Methods 6, 343-345 (2009).
67. Meisner, J. & Goldberg, J. B. The Escherichia coli rhaSR-PrhaBAD Inducible Promoter System Allows Tightly Controlled Gene Expression over a Wide Range in Pseudomonas aeruginosa. Appl. Environ. Microbiol. 82, 6715-6727 (2016).
68. Borges, A. L. et al. Bacteriophage Cooperation Suppresses CRISPR-Cas3 and Cas9 Immunity. Cell 174, 917-925.e10 (2018).
69. Nyerges, Á. et al. Directed evolution of multiple genomic loci allows the prediction of antibiotic resistance. Proc. Natl. Acad. Sci. 115, E5726-E5735 (2018).
70. Huynh, T. V., Dahlbeck, D. & Staskawicz, B. J. Bacterial blight of soybean: regulation of a pathogen gene determining host cultivar specificity. Science 245, 1374-1377 (1989).
71. Kropinski, A. M. Sequence of the Genome of the Temperate, Serotype-Converting, Pseudomonas aeruginosa Bacteriophage D3. J. Bacteriol. 182, 6066-6074 (2000).
72. Budzik, J. M., Rosche, W. A., Rietsch, A. & O'Toole, G. A. Isolation and Characterization of a Generalized Transducing Phage for Pseudomonas aeruginosa Strains PAO1 and PA14. J. Bacteriol. 186, 3270-3273 (2004).
73. Alikhan, N.-F., Petty, N. K., Ben Zakour, N. L. & Beatson, S. A. BLAST Ring Image Generator (BRIG): simple prokaryote genome comparisons. BMC Genomics 12, 402 (2011).

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

Informal Sequence Listing (partial)

SEQ ID NO: 1 (WT crRNA repeat sequence)
GTCGCGCCCCGCACGGGCGCGTGGATTGAAAC
SEQ ID NO: 2 (modified crRNA repeat sequence)
GTCGCCCGGCAAAACCGGGCGTGGATTGAAAC
SEQ ID NO: 3 (Cas3, Pseudomonas aeruginosa)
MDAEASDTHFFAHSTLKADRSDWQPLVEHLQAVARLAGEKAAFFGGGELAALAGL
LHDLGKYTDEFQRRIAGDAIRVDHSTRGAILAVERYGALGQLLAYGIAGHHAGLAN
GREAGERTALVDRLKGVGLPRLLEGWCVEIVLPERLQPPPLKARLERGFFQLAFLGR
MLFSCLVDADYLDTEAFYHRVEGRRSLREQARPTLAELRAALDRHLTEFKGDTPVN
RVRGEILAGVRGKASELPGLFSLTVPTGGGKTLASLAFALDHALAHGLRRVIYVIPFT
SIVEQNAAVFRRALGALGEEAVLEHHSAFVDDRRQSLEAKKKLNLAMENWDAPIVV
TTAVQFFESLFADRPAQCRKLHNIAGSVVILDEAQTLPLKLLRPCVAALDELALNYR
CSPVLCTATQPALQSPDFIGGLQDVRELAPEPQRLFRELVRVRIRTLGPLEDAALTEQI
ARREQVLCIVNNRRQARALYESLAELPGARHLTTLMCAKHRSSVLAEVRQMLKKGE
PCRLVATSLIEAGVDVDFPVVLRAEAGLDSIAQAAGRCNREGKRPLAESEVLVFAAA
NSDWAPPEELKQFAQAAREVMRLHPDDCLSMAAIERYFRILYWQKGAEELDAGNLL
GLIERGRLDGLPYETLATKFRMIDSLQLPVIIPFDDEARAALRELEFADGCAAIARRLQ
PYLVQMPRKGYQALREAGAIQAAAGTRYGEQFMALVNPDLYHHQFGLHWDNPAF
VSSERLCW*
SEQ ID NO: 4 (Cas5, Pseudomonas aeruginosa)
MAYGIRLMVWGERACFTRPEMKVERVSYDAITPSAARGILEAIHWKPAIRWVVDRI
QVLKPIRFESIRRNEVGGKLSAVSVGKAMKAGRTNGLVNLVEEDRQQRATTLLRDV
SYVIEAHFEMTDRAGADDTVGKHLDIFNRRARKGQCFHTPCLGVREFPASFRLLEEG
SAEPEVDAFLRGERDLGWMLHDIDFADGMTPHFFRALMRDGLIEVPAFRAAEDKA*
SEQ ID NO: 5 (Cas8, Pseudomonas aeruginosa)
MILSALNDYYQRLLERGEANISPFGYSQEKISYALLLSAQGELLDVQDIRLLSGKKPQ
PRLMSVPQPEKRTSGIKSNVLWDKTSYVLGVSAKGGERTQQEHESFKTLHRQILVGE
GDPGLQALLQFLDCWQPEQFKPPLFSEAMLDSNLVFRLDGQQRYLHETPAALALRT
RLLADGDSREGLCLVCGQRQPLARLHPAVKGVNGAQSSGASIVSFNLDAFSSYGKSQ
GENAPVSEQAAFAYTTVLNHLLRRDEHNRQRLQIGDASVVFWAQADTPAQVAAAE
STFWNLLEPPADDGQEAEKLRGVLDAVATGRPLHELDSLMEEGTRIFVLGLAPNTSR
LSIRFWAVDSLAVFTQHLAEHFRDMHLEPLPWKTEPAIWRLLYATAPSRDGRAKTE
DLLPQLAGEMTRAILTGSRYPRSLLANLIMRMRADGDVSGIRVALCKAVLAREARLS
GKIHQEELPMSLDKDASNPGYRLGRLFAVLEGAQRAALGDRVNATIRDRYYGAASS
TPATVFPILLRNTQNHLAKLRKEKPGLAVNLERDIGEIIDGMQSQFPRCLRLEDQGRF
AIGYYQQAQARFNRGPDSVE*
SEQ ID NO: 6 (Cas7, Pseudomonas aeruginosa)
MTAISNRYEFVYLFDVSNGNPNGDPDAGNMPRLDPETNQGLVTDVCLKRKIRNYVS
LEQESAPGYAIYMQEKSVLNNQHKQAYEALGIESEAKKLPKDEAKARELTSWMCKN
FFDVRAFGAVMTTEINAGQVRGPIQLAFATSIDPVLPMEVSITRMAVTNEKDLEKERT
MGRKHIVPYGLYRAHGFISAKLAERTGFSDDDLELLWRALANMFEHDRSAARGEM
AARKLIVFKHEHAMGNAPAHVLFGSVKVERVEGDAVTPARGFQDYRVSIDAEALPQ
GVSVREYL*
SEQ ID NO: 7 (I-C CRISPR-Cas3 repeat sequence, Legionella pneumophila)
GTCGCGCCCCGTGCGGGCGCGTGGATTGAAAC
SEQ ID NO: 8 (I-C CRISPR-Cas3 repeat sequence, Desulfovibrio vulgaris)
GTCGCCCCCCACGCGGGGGCGTGGATTGAAAC
SEQ ID NO: 9 (I-C CRISPR-Cas3 repeat sequence, Eggerthella lento)
GTCACTCCCCGCATGGGGAGTGCGGGTTGAAAT

What is claimed is:

1. A Cas3 protein variant comprising a sequence having at least 90% identity to SEQ ID NO: 3 and a D370A mutation.

2. The Cas3 protein variant of claim 1, wherein the Cas3 protein variant is a non-processive Cas3 helicase mutant.

3. The Cas3 protein variant of claim 1, wherein the Cas3 protein variant generates a smaller deletion at a target site than its wild-type counterpart.

4. The Cas3 protein variant of claim 3, wherein the target site is within a bacterial genome.

5. The Cas3 protein variant of claim 3, wherein the target site is within a phage genome.

6. A Cas3 protein variant comprising the sequence of SEQ ID NO: 3 and having a D370A mutation.

7. The Cas3 protein variant of claim 6, wherein the Cas3 protein variant is a non-processive Cas3 helicase mutant.

8. The Cas3 protein variant of claim 6, wherein the Cas3 protein variant generates a smaller deletion at a target site than its wild-type counterpart.

9. The Cas3 protein variant of claim 8, wherein the target site is within a bacterial genome.

10. The Cas3 protein variant of claim 8, wherein the target site is within a phage genome.

11. A method of inducing a deletion in a phage genome, the method comprising targeting the phage genome with the Cas3 protein variant of claim 1.

12. The method of claim 11, wherein the Cas3 protein variant is a non-processive Cas3 helicase mutant.

13. The method of claim 11, wherein the Cas3 protein variant generates a smaller deletion at a target site within the phage genome than its wild-type counterpart.

14. A method of inducing a deletion in a phage genome, the method comprising targeting the phage genome with the Cas3 protein variant of claim 6.

15. The method of claim 14, wherein the Cas3 protein variant is a non-processive Cas3 helicase mutant.

16. The method of claim 14, wherein the Cas3 protein variant generates a smaller deletion at a target site within the phage genome than its wild-type counterpart.
